SEQUENCE LISTING (4 sequences)

SEQ ID NO: 1 - protein, 8 aa, synthetic construct
GTAGLIGQ

SEQ ID NO: 2 - protein, 8 aa, synthetic construct
GTAGLIGQ

SEQ ID NO: 3 - protein, 5 aa, synthetic construct
KKKKK

SEQ ID NO: 4 - protein, 4 aa, synthetic construct
RGDS

(19) United States
(12) Patent Application Publication
(10) Pub. No.: US 2024/0199698 A1
(43) Pub. Date: Jun. 20, 2024

(54) PEPTIDE AMPHIPHILE COMPOSITIONS AND METHODS OF USE THEREOF

(71) Applicants: UAB RESEARCH FOUNDATION, Birmingham, AL (US); Endomimetics LLC, BIRMINGHAM, AL (US)

(72) Inventors: HO-WOOK JUN, Birmingham, AL (US); BRIGITTA BROTT, Birmingham, AL (US); XIXI ZHANG, Birmingham, AL (US); JUN CHEN, Birmingham, AL (US); JENNIFER SHERWOOD, Birmingham, AL (US); PATRICK HWANG, Birmingham, AL (US)

(21) Appl. No.: 18/482,740

(22) Filed: Oct. 6, 2023

Related U.S. Application Data
(60) Provisional application No. 63/378,933, filed on Oct. 10, 2022.

(51) Int. Cl. C07K 7/08; A61K 31/165; A61K 31/7036; A61K 38/14; A61K 45/06; A61P 9/10; A61P 29/00

(52) U.S. Cl. CPC C07K 7/08; A61K 31/165; A61K 31/7036; A61K 38/14; A61K 45/06; A61P 9/10; A61P 29/00

(57) ABSTRACT

Disclosed are peptide amphiphiles comprising a hydrophilic peptide sequence and a hydrophobic tail, wherein the hydrophobic tail comprises a moiety having a substituted C5 alkyl chain, wherein the hydrophilic peptide sequence comprises a nitric oxide producing donor sequence. In some aspects, the hydrophilic peptide sequence comprises a degrading sequence (DS), wherein the degrading sequence (DS) comprises the amino acid sequence GTAGLIGQ (SEQ ID NO:1) wherein the DS comprises one or more amino acid substitutions. Also disclosed are compositions, liposomes, gels, and medical devices comprising the peptide amphiphiles and methods of use thereof.

### CROSS-REFERENCE TO RELATED APPLICATIONS

This application claims the benefit of U.S. Provisional Patent Application No. 63/378,933, filed Oct. 10, 2022, which is incorporated by reference herein in its entirety.

### STATEMENT REGARDING FEDERALLY SPONSORED RESEARCH

This invention was made with government support under Grant Number 1R01HL163802-01 awarded by the National Institutes of Health. The government has certain rights in this invention.

### REFERENCE TO SEQUENCE LISTING

The Sequence Listing submitted Feb. 20, 2024 as an xml file named “21085.0194U2.xml,” created on Feb. 20, 2024, and having a size of 4,485 bytes is hereby incorporated by reference pursuant to 37 C.F.R. § 1.52(e)(5).

### BACKGROUND

Recently, numerous studies have investigated the co-delivery of different therapeutic drugs, including proteins, hydrophobic compounds, and hydrophilic drugs, to improve cancer therapy, stimulate cardiac repair, and prevent atherosclerotic foam cell formation. In addition, it has been reported that the co-delivery of multiple therapeutics offers an excellent opportunity to target different mechanisms for treating diseases, thereby possibly maximizing the therapeutic efficacy, overcoming drug resistance mechanisms, inducing synergistic effects, or decreasing drug toxicity and side effects.

Nitric oxide (NO) plays a vital role in biology, physiology, and pathophysiology, a key signaling molecule in cardiovascular homeostasis. Abnormal NO delivery is strongly associated with the development of CVD. In addition, NO is an endothelium-derived relaxing factor produced in the vascular endothelial cell (VEC), which can regulate vascular tone, lower lipid levels, and inhibit adhesion molecule expressions, platelet aggregation, and vascular smooth muscle cell (VSMC) proliferation. In addition, the endothelium plays an essential role in regulating AVF development. Endothelial-derived NO is a crucial vasodilator and signaling molecule in vascular remodeling. Moreover, NO demonstrates anti-inflammatory effects in some studies. However, unlike the traditional chemical drugs, the metabolites converted from NO are always harmless. Despite NO's beneficial functions, its use in the clinic is limited due to its high reactivity and short diffusion distance. Several groups have synthesized exogenous NO donors, such as S-nitrosothiols (RSNOs), n-diazeniumdiolates (NONOates), and nitrosamines, which can generate NO under specific conditions. However, these exogenous NO donors are commonly administered by systemic delivery, and they suffer from short half-lives and ineffective delivery. In addition, burst release may occur when the NO is released from these exogenous NO donors, possibly resulting in adverse local toxicity. To solve those problems, drug delivery systems have been developed, including nanomaterials such as liposomes, micelles, peptide amphiphiles, and polymer nanoparticles. Moreover, the combination of NO donors with nanomaterials has emerged as a great strategy for spatial-temporal NO release directly at the target site.

### BRIEF SUMMARY

Described herein are compositions comprising NO-releasing nanomaterials and other therapeutic drugs for other disease conditions such as cardiovascular disease, amputation, and arteriovenous fistulas maturation failure. As embodied and broadly described herein, described herein are NO-releasing peptide amphiphiles and NO-releasing liposomes for producing the compositions comprising NO-releasing nanomaterials and therapeutic drugs for treating various diseases such as cardiovascular disease, amputation, and AVF maturation failure. In one embodiment, the disclosed composition comprises NO-releasing peptide amphiphiles and drugs (or the liposomal drugs), such as sirolimus, everolimus, paclitaxel, statins and colchicine, for treating or preventing atherosclerosis. In other embodiment, the disclosed composition comprises NO-releasing peptide amphiphile and antibiotics (or the liposomal antibiotics) for improving amputation treatment. In another embodiment, the disclosed compositions can take the form of gel for administering in the subject (such as a human) to address AVF maturation failure. In one embodiment, the disclosed composition can be ultrasonically sprayed onto medical devices such as stents, balloons, or percutaneous osseointegrated prostheses. In another embodiment, the present invention includes the methods of making and using NO-releasing peptide amphiphile and NO-releasing liposomes.

As NO and the therapeutic drugs, such as sirolimus, everolimus, paclitaxel, colchicine, and statins or their liposomal drugs, may act through different molecular mechanisms for treating atherosclerosis, it is possible that combining these agents may potentiate each other's therapeutic effect by suppressing smooth muscle and inflammatory cell proliferation as well as reducing inflammation while improving endothelialization. The combination of NO and therapeutic drugs (or their liposomal drugs), represents a new therapeutic combination that may be more efficacious against inflammation, thrombosis, restenosis/neointimal thickening than therapeutic drugs alone. The combination of NO and therapeutic drugs may be efficacious for cardiovascular diseases such as atherosclerosis. Moreover, the combination of NO with sirolimus (or its liposomal drug) may minimize the site effects from sirolimus for treating atherosclerosis. It would also be advantageous to coat the implantable medical devices using the disclosed composition comprising NO and drug agents to treat disease and minimize or substantially eliminate a living organism's reaction to the implantation of the medical device. In certain circumstances, it may be advantageous to coat the implantable medical devices using the disclosed compositions comprising NO and therapeutic drugs, which may promote healing and endothelialization of the medical device.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIG. 1 shows DLS analysis of the size of PA-05-K5-NO (0.25 wt %) at Day 0.

FIGS. 2A and 2B show the TEM image of PA-05-K5-NO encapsulated liposomes made with DPPC, DOTAP, DSPE-PEG, and CHOL: FIG. 2A shows low magnification TEM image of PA-05-K5-NO encapsulated liposomes. FIG. 2B shows high magnification TEM image of PA-05-K5-NO encapsulated liposomes.

FIGS. 3A and 3B show the DLS analysis of the size of PA-05-K5-NO encapsulated liposomes: FIG. 3A shows size of PA-05-K5-NO encapsulated liposomes on Day 0 and FIG. 3B shows size of PA-05-K5-NO encapsulated liposomes on Day 7.

FIG. 4 shows the NO-releasing profile of PA-05-K5-NO and PA-05-K5-NO encapsulated liposomes.

FIGS. 5A-B show TEM analysis of PA-C16-YK5-NO encapsulated liposomes (DPPC:DOTAP:DSPE-PEG:CHOL) At Day 5: FIG. 5A shows low magnification TEM image of PA-C16-YK5-NO encapsulated liposomes. FIG. 5B shows high magnification TEM image of PA-C16-YK5-NO encapsulated liposomes

FIGS. 6A-C show the DLS analysis of PA-C16-K5-NO encapsulated liposomes (DPPC:DOTAP:DSPE-PEG:CHOL) at different times: FIG. 6A shows size of PA-C16-K5-NO encapsulated liposomes on Day 0. FIG. 6B shows size of PA-C16-K5-NO encapsulated liposomes on Day 5. FIG. 6C shows size of PA-C16-K5-NO encapsulated liposomes on Day 7.

FIGS. 7A-K show the characterization of empty and miRNA loaded liposomes: FIG. 7A shows the DLS analysis of liposomes after freeze-thaw. Liposomes were stored at 4, −30, −80° C. with 0, 1, 5, or 20% sucrose as a cryoprotectant. FIG. 7B shows DLS analysis of long-term liposome stability at 0, 7, 28, 56 days (See Table 1). FIGS. 7C-K shows representative TEM images of unloaded/loaded liposomes at 0, 7, 28 days

FIGS. 8A-F show the microRNA146a-releasing liposome transfection. FIG. 8A shows the bright field image of HAECs transfected with fluorescent tagged miR-146a loaded liposomes. FIG. 8B shows the fluorescent image of HAECs transfected with fluorescent tagged miR-146a loaded liposomes. FIG. 8C shows the bright field image of SMCs transfected with fluorescent tagged microRNA146a-releasing liposome. FIG. 8D shows the fluorescent image of SMCs transfected with fluorescent tagged microRNA146a-releasing liposome. FIG. 8E shows the bright field image of macrophages (differentiated U937 cells) with fluorescent tagged microRNA146a-releasing liposome. FIG. 8F shows the fluorescent image of macrophages (differentiated U937 cells) transfected with fluorescent tagged microRNA146a-releasing liposome.

FIGS. 9A-C show the analytic gating strategy of flow cytometry for microRNA146a-releasing liposome (146a-lipo) transfected cells. FIG. 9A shows the flow cytometry data of human aortic smooth muscle cells after transfection/treatment of free microRNA146a or microRNA146a-releasing liposome. FIG. 9B shows the flow cytometry data of PMA-differentiated U937 cells, after transfection/treatment of free microRNA146 or microRNA146a-releasing liposome. FIG. 9C shows the flow cytometry data of human aortic endothelial cells after transfection/treatment of free microRNA146a-releasing liposome.

FIGS. 10A-E show the microRNA-146a-liposomes effect on foam cell formation. FIGS. 10A-FIG 10D show the fluorescent images of BODIPY stained differentiated U937 cells treated with LPS, ox-LDL, neg-lipo, and microRNA-146a-liposome. FIG. 10E shows the Analysis of foam cell formation based on BODIPY stain intensity. Differentiated U937 cells were treated with LPS, ox-LDL, emp-lipo, neg-lipo, and MicroRNA-146a-liposomes then stained with BODIPY.

FIGS. 11A-K show PA-C16-YK5-NO nanomatrix coating could promote the endothelial functions in vitro. FIG. 11A show immunostaining of ICAM-1 expressed by hAECs on BMS stent strut. FIG. 11B show immunostaining of ICAM-1 expressed by hAECs on DES stent strut. FIG. 11C shows immunostaining of ICAM-1 expressed by hAECs on PA-C16-YK5-NO stent strut. FIG. 11D shows the quantification of ICAM-1 fluorescent intensity on stent strut. FIG. 11E shows DF-FM staining of hAEC on BMS stent strut. FIG. 11F shows DF-FM staining of hAEC on DES stent strut. FIG. 11G shows DF-FM staining of hAEC on PA-C16-YK5-NO stent strut. FIG. 11H shows quantification of DAF-FM fluorescent intensity on stent strut FIG. 11I-K show gene expression of CD34, eNOS, and PECAM-1. (*p<0.05) (FI- fluorescence intensity)

FIGS. 12A-B show PA-C16-YK5-NO nanomatrix coating could promote the transition of SMC to contractile phenotype in vitro. FIGS. 12A and 12B show gene expression of MYH11 and PDGF-B of hAoSMC on stent strut. (*p<0.05)

FIGS. 13A-E show PA-C16-YK5-NO nanomatrix coating could regulate inflammation in vitro. FIGS. 13A, 13B, and 13C show Calcein AM Blue stained monocytes adhered on stent strut. FIG. 13D shows quantification of the fluorescent intensity from Calcein AM Blue stained monocytes on stent strut. FIG. 13E shows inflammatory cytokine secretion from VDLs after being treated with BMS, DES or PA-C16-YK5-NO coated stents on day 7. FIG. 13F shows NOX4 expression of cells on stent strut. FIG. 13G shows quantification of oxidative stress using DCFH-DA assay. (*p<0.05) (FI- fluorescence intensity)

FIGS. 14A-F show PA-C16-YK5-NO nanomatrix coating could administer foam cell formation, ECM remodeling, and calcification in vitro. FIG. 14A shows quantification of fluorescent intensity of BODIPY stained foam cell. FIGS. 14B-F show gene expression of RUNX2, BMP2, MMP2, CAL3A1, and COL17A1.

FIGS. 15A-F shows PA-C16-YK5-NO nanomatrix coating could accelerate stent coverage in vivo. FIGS. 15A-F show representative SEM images of BMS, DES, and PA-C16-YK5-NO coated stent at day 4 and 28. FIG. 15G shows quantification of stent coverage on day 4 and 28. (*p<0.05)

FIGS. 16A-I show PA-C16-YK5-NO nanomatrix coating could improve endothelialization while suppress restenosis, and inflammation in vivo. FIGS. 16A-B show representative SEM images of BMS on day 4 and 28. FIGS. 16D-E show representative SEM images of BDES on day 4 and 28. FIGS. 16G- and 6H show representative SEM images of PA-C16-YK5-NO coated stent on day 4 and 28. FIG. 16C, 16F, and 161 show representative images for H&E staining of arteries after stented with BMS, DES and PA-C16-YK5-NO coated stents on day 28. (*p<0.05)

FIGS. 17A-C show the sirolimus (FIG. 17A) TEM image of liposome-encapsulated sirolimus. The scale bar is 500 nm. (FIG. 17B)-(FIG. 17C) SEM images of the prohealing PA-C16-YK5-NO nanomatrix coated stent before and after clinician's handling and balloon inflation.

FIGS. 18A-B shows the effect of colchicine on (FIG. 18A) diseased EC function and (FIG. 18B) foam cell formation.

FIG. 19 shows the proliferation of hAECs proliferation. Sirolimus treatment only (blue) and dual Sirolimus+NO (10⁻⁹ M) treatment (red).

FIGS. 20A-B shows the effect of (FIG. 20A) sirolimus and PA-C16-YK5-NO dual treatment on hAoSMC proliferation using PCNA assay and (FIG. 20B) calcein-Am staining.

FIG. 21 shows the effect of the sirolimus and PA-C16-YK5-NO dual treatment on hAoSMC apoptosis using caspase 3/7.

FIGS. 22A-B show sustained release of everolimus. FIG. 22A demonstrates the percentage of total Everolimus released from the liposomes while FIG. 22B represents the mass of released Everolimus.

FIG. 23 shows the distribution of sizes via a logarithmic scale in the Everolimus-releasing liposomes.

### DETAILED DESCRIPTION

The disclosed method and compositions may be understood more readily by reference to the following detailed description of particular embodiments and the Example included therein and to the Figures and their previous and following description.

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. Thus, if a class of molecules A, B, and C are disclosed as well as a class of molecules D, E, and F and an example of a combination molecule, A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

### A. Definitions

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a ”, “an”, and “the” include plural reference unless the context clearly dictates otherwise. Thus, for example, reference to “a peptide amphiphile” includes a plurality of such peptide amphiphiles, reference to “the peptide amphiphile” is a reference to one or more peptide amphiphiles and equivalents thereof known to those skilled in the art, and so forth.

As used herein, the term “peptide amphiphile” refers to a peptide compound possessing both a hydrophilic portion (e.g., a hydrophilic peptide sequence moiety) and a hydrophobic tail (e.g., a hydrocarbon moiety). One property typically associated with a peptide amphiphile can be self-assembly.

As used herein, the term “hydrophilic peptide sequence” refers to a peptide residue sequence having hydrophilicity properties relative to a hydrocarbon moiety. A hydrophilic peptide sequence can comprise one or more functional peptide sequences (e.g., degradable peptide sequences, nitric oxide donors, and/or cell adhesive ligands).

As used herein, the term “degradation sequence” refers to a sequence of peptide residues that can be degraded by enzymes or hydrolysis under biological conditions.

As used herein, the term “cell adhesive sequence” refers to a sequence of peptide residues capable of operation as adhesive ligands for cells. In one aspect, due to the amphiphilic characteristic of peptide amphiphiles, the disclosed cell adhesive sequences are exposed at the exterior surface of a nanofiber assembly; thus, such cell adhesive sequence can be available for interaction with one or more cells. One example is an “endothelial cell adhesive sequence,” which refers to a peptide sequence that supports endothelial cell adhesion, spreading, migration, and/or growth.

As used herein, the term “nitric oxide producing donor sequence” refers to a peptide residue (e.g., lysine (K) or cysteine (C)) or sequence of peptide residues (e.g., polylysine (KKKKK) or polycysteine (CCCCC)) capable of reversibly binding nitric oxide gas, or equivalent thereof) as a complex (e.g., diazoniumdiolates). Thus, the peptide or sequence can serve as a reservoir for nitric oxide gas and can selectively release nitric oxide over time. It is understood that the term can include other nitric oxide donors, for example, any peptide sequences containing cysteine or amine groups.

As used herein, the term “NO-releasing nanomaterials” refers to materials whose size is less than 1 micron and can generate and release nitric oxide to their surrounding environment.

As used herein, the term “subject” refers to a target of administration. The subject of the herein disclosed methods can be a vertebrate, such as a mammal, a fish, a bird, a reptile, or an amphibian. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. A patient refers to a subject afflicted with a disease or disorder. The term “patient” includes human and veterinary subjects.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used herein, the term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the term “pharmaceutically active agent” includes a “drug” or an “antibiotic” and means a molecule, group of molecules, complex or substance administered to an organism for diagnostic, therapeutic, preventative medical, or veterinary purposes. This term includes externally and internally administered topical, localized and systemic human and animal pharmaceuticals, treatments, remedies, nutraceuticals, cosmeceuticals, biologicals, devices, diagnostics and contraceptives, including preparations useful in clinical and veterinary screening, prevention, prophylaxis, healing, wellness, detection, imaging, diagnosis, therapy, surgery, monitoring, cosmetics, prosthetics, forensics and the like. This term may also be used in reference to agriceutical, workplace, military, industrial and environmental therapeutics or remedies comprising selected molecules or selected nucleic acid sequences capable of recognizing cellular receptors, membrane receptors, hormone receptors, therapeutic receptors, microbes, viruses or selected targets comprising or capable of contacting plants, animals and/or humans. This term can also specifically include nucleic acids and compounds comprising nucleic acids that produce a bioactive effect, for example deoxyribonucleic acid (DNA) or ribonucleic acid (RNA). Pharmaceutically active agents include the herein disclosed categories and specific examples. It is not intended that the category be limited by the specific examples. Those of ordinary skill in the art will recognize also numerous other compounds that fall within the categories and that are useful according to the invention.

As used herein, the term “peptide amphiphile” refers to a peptide compound possessing both a hydrophilic portion (e.g., a hydrophilic peptide sequence moiety) and a hydrophobic portion (e.g., a hydrocarbon moiety). One property typically associated with a peptide amphiphile can be self-assembly.

As used herein, the term “self-assembling” refers to the characteristic of a plurality of molecules of a compound in which a disordered system forms a more organized structure or pattern due to specific, local interactions among the molecules themselves without external force. In one aspect, peptide amphiphiles can be self-assembling. In a further aspect, peptide amphiphiles can self-assemble into nanofibers or nanoparticles.

As used herein, the terms “delivery” and “delivering” refer to carrying a therapeutic compound or the disclosed compositions to a particular subject site. Delivery can be located in a specific particular location

As used herein, the term “drug delivery systems” refers to materials developed by engineering technologies for delivering therapeutic drugs to specific locations of the subject. These materials are primary particles, such as liposomes, polymeric nanoparticles, micelles, and inorganic nanoparticles.

As used herein, the term “liposomes” refers to a closed vesicle with an internal phase separated by at least one lipid bilayer, whose diameter ranges from 20 nm to 400 nm. One property associated with liposome is that its internal phase and lipid bilayer can hold water-soluble and lipophilic materials. The internal liposome phase means the aqueous region enclosed in the liposome's lipid bilayer. It is also called the “internal water phase” and “liposome internal water phase.” In the present invention, liposomes refer to small single-membrane liposomes (SUV: small unilamellar vesicle).

As used herein, the term “hydrogel” refers to two or multi-component water-swollen biomaterials consisting of a three-dimensional network of polymers and water.

“Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

### B. Peptide Amphiphiles

A peptide amphiphile (PA) is a molecule that possesses an amphiphilic structure typically composed of a hydrophilic peptide sequence and a hydrophobic tail. Due to the amphiphilic nature, peptide amphiphiles can self-assemble into various structures, including sheets, spheres, rods, or disks, depending on the charge and environment (pH and salt). In addition, PA is reported to self-assemble into micelles when the PA concentration is above its critical micelle concentration. Moreover, if the hydrophilic head group of the PA is bulkier than the hydrophobic tail, cylindrical micelles known as nanofibers are formed. The driven force for self-assembly is from amino acids of the PA. For instance, the negatively charged amino acids in the backbone of the PA improve the PA's solubility. However, when the negative charge of the amino acids is eliminated by lowing the pH of the PA solution or introducing the divalent ions into the PA system, PA self-assembles into specific stable structures. In addition, it should be noted that the presence of hydrogen bonds among the amino acids of the backbone of the PA leads to the formation of cylindrical structures; otherwise, spherical structures would form.

Disclosed are peptide amphiphiles comprising a hydrophilic peptide sequence and a hydrophobic tail. In some aspects, the length of the hydrophilic peptide sequence and hydrophobic tail can be selected such that the peptide amphiphile maintains the ability to self-assemble into a nanomatrix. Thus, for example, the length of the hydrophobic tail can be increased to accommodate for an increased length in the hydrophilic peptide. The skilled artisan can use routine skill to screen for the ability of a peptide amphiphile with a selected hydrophilic peptide and a selected hydrophobic tail to self-assemble into a nanomatrix.

Disclosed are peptide amphiphiles comprising a hydrophilic peptide sequence and a hydrophobic tail, wherein the hydrophobic tail comprises a moiety having an optionally substituted C4-C6 alkyl chain, wherein the hydrophilic peptide sequence comprises a nitric oxide (NO) producing donor sequence. Thus, disclosed are peptide amphiphiles comprising a hydrophilic peptide sequence and a hydrophobic tail, wherein the hydrophobic tail comprises a moiety having a substituted C5 alkyl chain, wherein the hydrophilic peptide sequence comprises a nitric oxide (NO) producing donor sequence.

In some aspects, the nitric oxide producing donor sequence comprises the amino acid sequence Lys-Lys-Lys-Lys-Lys (KKKKK; K5; SEQ ID NO:3). In some aspects, one or more of the Lys residues comprise a pendant amine group. In some aspects, the pendant amine groups can react with nitric oxide to form a diazeniumdiolate-modified peptide amphiphile. In some aspects, a diazeniumdiolate is a NO donor and comprises one or more molecules of NO.

In some aspects, the nitric oxide producing donor sequence can comprise the diazeniumdiolate-modified amino peptide [K[N(O )NO⁻]]ₙ, wherein “n” is from 1 to 20. In some aspects, n is 1, 2, 3, 4, 5, 6, 7, 8, 9, 10, 11, 12, 13, 14, 15, 16, 17, 18, 19, 20 or higher. Thus, the nitric oxide producing donor sequence can comprise the diazeniumdiolate-modified peptide [K[N(O )NO⁻]]₅.

Disclosed herein are peptide amphiphiles comprising a hydrophilic peptide sequence and a hydrophobic tail, wherein the hydrophobic tail comprises a moiety having a substituted C5 alkyl chain, wherein the hydrophilic peptide sequence comprises a nitric oxide producing donor sequence.

Disclosed herein are peptide amphiphiles comprising a hydrophilic peptide sequence and a hydrophobic tail, wherein the hydrophobic tail comprises a moiety having a substituted C5 alkyl chain, wherein the hydrophilic peptide sequence comprises a nitric oxide producing donor sequence wherein the nitric oxide producing donor sequence comprises the amino acid sequence Lys-Lys-Lys-Lys-Lys (KKKKK) (K5; SEQ ID NO:3).

Disclosed herein are peptide amphiphiles comprising a hydrophilic peptide sequence and a hydrophobic tail, wherein the hydrophobic tail comprises a moiety having a substituted C5 alkyl chain, wherein the hydrophilic peptide sequence comprises a nitric oxide producing donor sequence wherein the nitric oxide producing donor sequence comprises the amino acid sequence Lys-Lys-Lys-Lys-Lys (KKKKK) (K5; SEQ ID NO:3) and wherein one or more of the Lys residues comprise a pendant amine group.

Disclosed herein are peptide amphiphiles comprising a hydrophilic peptide sequence and a hydrophobic tail, wherein the hydrophobic tail comprises a moiety having a substituted C16 alkyl chain, wherein the hydrophilic peptide sequence comprises a nitric oxide producing donor sequence comprising an amino acid sequence Lys-Lys-Lys-Lys-Lys (KKKKK; SEQ ID NO:3) and a degrading sequence (DS) comprising an amino acid sequence Gly-Thr-Ala-Gly-Leu-ILE-Gly-Gln (GTAGLIGQ; SEQ ID NO:1), wherein one or more of the lysine residues comprise a pendant amine group. In some aspects, the peptide amphiphiles comprise one or more molecules of nitric oxide bound to at least one pendant amine group.

Disclosed herein are mixtures of peptide amphiphiles. In an aspect, disclosed herein is a mixture of two peptide amphiphiles, wherein the first peptide amphiphile comprises a hydrophilic peptide sequence and a hydrophobic tail, wherein the hydrophobic tail comprises a moiety having a substituted C16 alkyl chain, wherein the hydrophilic peptide sequence comprises an endothelial cell-adhesive sequence (CA) comprising an amino acid sequence Tyr-Ile-Gly-Ser-Arg (YIGSR; SEQ ID NO:2) and a degrading sequence (DS) comprising an amino acid sequence Gly-Thr-Ala-Gly-Leu-ILE-Gly-Gln (GTAGLIGQ; SEQ ID NO:1), and wherein the second peptide amphiphile comprises comprises a hydrophilic peptide sequence and a hydrophobic tail, wherein the hydrophobic tail comprises a moiety having optionally substituted C16 alkyl chain, wherein the hydrophilic peptide sequence comprises a nitric oxide producing donor sequence comprising an amino acid sequence Lys-Lys-Lys-Lys-Lys (KKKKK) (K5; SEQ ID NO:3) and also comprises a degrading sequence (DS) comprising an amino acid sequence Gly-Thr-Ala-Gly-Leu-ILE-Gly-Gln (GTAGLIGQ; SEQ ID NO:1). In some aspects, the peptide amphiphiles comprise one or more molecules of nitric oxide bound to at least one pendant amine group.

Disclosed herein are peptide amphiphiles comprising a hydrophilic peptide sequence and a hydrophobic tail, wherein the hydrophobic tail comprises a moiety having a substituted C5 alkyl chain, wherein the hydrophilic peptide sequence comprises a nitric oxide producing donor sequence, comprising the amino acid sequence Lys-Lys-Lys-Lys-Lys (KKKKK; SEQ ID NO:3) and wherein one or more of the Lys residues comprise a pendant amine group. In some aspects, the peptide amphiphiles comprise one or more molecules of nitric oxide bound to at least one pendant amine group.

Disclosed herein is peptide amphiphile 1 (PA-C16-DS) that comprises a hydrophilic peptide sequence and a hydrophobic tail, wherein the hydrophobic tail comprises a moiety having optionally substituted C16 alkyl chain, wherein the hydrophilic peptide sequence comprises a degrading sequence (DS) comprising an amino acid sequence Gly-Thr-Ala-Gly-Leu-ILE-Gly-Gln (GTAGLIGQ; SEQ ID NO:1). Besides peptide amphiphile 1, peptide amphiphile 2 comprises a hydrophilic peptide sequence and a hydrophobic tail, wherein the hydrophilic peptide sequence comprises a degradation sequence comprising an amino acid sequence of Gly-Thr-Ala-Gly-Leu-ILE-Gly-Gln (GTATGIQ; SEQ ID NO:1) and one cell-adhesive sequence comprising the amino acid sequence Tyr-Ile-Gly-Ser-Arg (YIGSR; SEQ ID NO:2). The adhesive sequence is an endothelial cell adhesive sequence that does not bind to smooth muscle cells and/or platelets. Peptide amphiphile 3 (PA-C16-1(5) comprises a hydrophilic peptide sequence and a hydrophobic tail, wherein the hydrophobic tail comprises a moiety having optionally substituted C16 alkyl chain, wherein the hydrophilic peptide sequence comprises a nitric oxide producing donor sequence comprising an amino acid sequence Lys-Lys-Lys-Lys-Lys (KKKKK) (K5; SEQ ID NO:3) and also comprises a degrading sequence (DS) comprising an amino acid sequence Gly-Thr-Ala-Gly-Leu-ILE-Gly-Gln (GTAGLIGQ; SEQ ID NO:1), wherein herein one or more of the lysine residues comprise pendant amine groups that react with NO to form a diazeniumdiolate-modified peptide amphiphile, named as peptide amphiphile 4 (PA-C16-K5-N0). Particularly, peptide amphiphile 4 can release NO to promote the homing of satellite endothelial progenitor cells, inhibiting restenosis and thrombosis. Peptide amphiphile 4 comprises a hydrophilic peptide and a hydrophobic tail, wherein the hydrophilic peptide comprises a nitric oxide producing donor sequence comprising an amino acid sequence Lys-Lys-Lys-Lys-Lys (SEQ ID NO:3: KKKKK) and a degradation sequence comprising an amino acid sequence of GTAGLIGQ. The hydrophobic tail comprises a moiety having optionally substituted C16 alkyl chain. In some aspect, the peptide amphiphile 4 can be PA-KKKKK-GTAGLIGQ-NO or PA-GTAGLIGQ-KKKKK-NO, wherein its C16 chain conncects to Q or K of the hydrophilic sequence KKKKK-GTAGLIGQ. Peptide amphiphile 3 (PA-C16-K5) has the same component as peptide amphiphile 4 but without NO conjugation. In addition to peptide amphiphile 4, peptide amphiphile6 (PA-C16-YK5-NO), can release NO, which comprises a combination of peptide amphiphile 2 (PA-C16-CA) and peptide amphiphile 4 (PA-C16-K5-NO) that can self-assemble into nanofibers. The NO-releasing peptide amphiphile 6 is regarded as an endothelium mimicking nanomatrix. The endothelium mimicking nanomatrix comprising peptide amphiphile 2 (PA-C16-CA) and peptide amphiphile 4 (PA-C16-K5-NO) can be present in the nanofibers at a ratio of from about 1:20 to about 20:1, including about 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, or 1:20. Peptide amphiphile 5 (PA-C16-YK) comprises peptide amphiphile 2 (PA-C16-CA) and peptide amphiphile 3 (PA-C16-K5) are at a molar ratio of from about 1:20 to about 20:1, including about 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, or 1:20. Peptide amphiphile 5 (PA-C16-YK) has the same component as peptide amphiphile 6 but without NO conjugation. Another NO-releasing peptide amphiphile, peptide amphiphile 8, comprises a hydrophilic peptide sequence that only comprises a nitric oxide producing donor sequence comprising an amino acid sequence KKKKK. It also comprises a hydrophobic tail from the low alkyl group that comprises a moiety having optionally substituted C5. In some respects, peptide amphiphiles 4,6 and 8 can be loaded into drug delivery systems to form NO-releasing nanomaterials, wherein the drug delivery systems are selected from liposomes, polymeric nanoparticles, micelles, and inorganic nanoparticles.

Disclosed herein is peptide amphiphile 9 (PA-C16-K5-YIGSR) comprising a hydrophilic peptide sequence and a hydrophobic tail, wherein the hydrophobic tail comprises a moiety having an optionally substituted C16 alkyl chain, wherein the hydrophilic peptide sequence comprises a degrading sequence (DS) comprising an amino acid sequence Gly-Thr-Ala-Gly-Leu-ILE-Gly-Gln (GTAGLIGQ; SEQ ID NO:1), and further comprises a nitric oxide producing donor sequence comprising an amino acid sequence Lys-Lys-Lys-Lys-Lys (KKKKK) (K5; SEQ ID NO:3) and one cell adhesive sequence comprising the amino acid sequence Tyr-Ile-Gly-Ser-Arg (YIGSR; SEQ ID NO:2). In some aspects, the peptide amphiphile 9 can be, PA-KKKKK-GTAGLIGQ-YIGSR or PA-GTAGLIGQ-KKKKK-YIGSR, with its C16 chain connected to the hydrophilic sequence with a sequence order of KKKKK-GTAGLIGQ-YIGSR or GTAGLIGQ-KKKKK-YIGSR.

Also disclosed herein is peptide amphiphile 10 (PA-C16-K5-YIGSR-NO) comprising a hydrophilic peptide sequence and a hydrophobic tail, wherein the hydrophobic tail comprises a moiety having an optionally substituted C16 alkyl chain, wherein the hydrophilic peptide sequence further comprises a degrading sequence (DS) comprising an amino acid sequence Gly-Thr-Ala-Gly-Leu-ILE-Gly-Gln (GTAGLIGQ; SEQ ID NO:1), and further comprises a nitric oxide producing donor sequence comprising an amino acid sequence Lys-Lys-Lys-Lys-Lys (KKKKK) (K5; SEQ ID NO:3) and one cell adhesive sequence comprising the amino acid sequence Tyr-Ile-Gly-Ser-Arg (YIGSR; SEQ ID NO:2), wherein one or more of the Lys residues of the peptide amphiphile comprises a pendant amine group, wherein one or more molecules of nitric oxide is bound to at least one pendant amine group. In some aspects, peptide amphiphile 10 can release NO. In some aspects, the peptide amphiphile 10 can be, PA-KKKKK-GTAGLIGQ-YIGSR-NO, or PA-GTAGLIGQ-KKKKK-YIGSR-NO, with its C16 chain connected to the hydrophilic sequence with a sequence order of KKKKK-GTAGLIGQ-YIGSR, or GTAGLIGQ-KKKKK-YIGSR.

Also disclosed herein is peptide amphiphile 11 (PA-C16-K5-RGDS) comprising a hydrophilic peptide sequence and a hydrophobic tail, wherein the hydrophobic tail comprises a moiety having an optionally substituted C16 alkyl chain, wherein the hydrophilic peptide sequence comprises a degrading sequence (DS) comprising an amino acid sequence Gly-Thr-Ala-Gly-Leu-ILE-Gly-Gln (GTAGLIGQ; SEQ ID NO:1), and further comprises a nitric oxide producing donor sequence comprising an amino acid sequence Lys-Lys-Lys-Lys-Lys (KKKKK) (K5; SEQ ID NO:3) and one cell adhesive sequence comprising the amino acid sequence Arg-Gly-Asp-Ser (RGDS; SEQ ID NO:5).

Also disclosed herein is peptide amphiphile 12 (PA-C16-K5-RGDS-NO) comprising a hydrophilic peptide sequence and a hydrophobic tail, wherein the hydrophobic tail comprises a moiety having an optionally substituted C16 alkyl chain, wherein the hydrophilic peptide sequence further comprises a degrading sequence (DS) comprising an amino acid sequence Gly-Thr-Ala-Gly-Leu-ILE-Gly-Gln (GTAGLIGQ; SEQ ID NO:1), and further comprises a nitric oxide producing donor sequence comprising an amino acid sequence Lys-Lys-Lys-Lys-Lys (KKKKK) (K5; SEQ ID NO:3) and one cell adhesive sequence comprising the amino acid sequence Arg-Gly-Asp-Ser (RGDS; SEQ ID NO:5), wherein one or more of the Lys residues of the peptide amphiphile comprises a pendant amine group, wherein one or more molecules of nitric oxide is bound to at least one pendant amine group.

It is understood that each lysine residue can be a donor for two nitric oxide molecules. The number of lysine residues or diazeniumdiolate-modified lysine residues can therefore be selected based on the amount of NO desired. This can further be regulated by the artisan by selecting the amount or concentration of the peptide amphiphiles comprising these lysine residues.

The hydrophobic tail can comprise a moiety having an optionally substituted C4 or larger alkyl chain. Thus, the hydrophobic tail can comprise a moiety having an optionally substituted C5 to C28 or larger alkyl chain. Thus, the hydrophobic tail can comprise a moiety having an optionally substituted C10 to C25 or larger alkyl chain. Thus, the hydrophobic tail can comprise a moiety having an optionally substituted C4, C5, C6, C7, C8, C9, C10, C11, C12, C13, C14, C15, C16, C17, C18, C19, C20, C21, C22, C23, C24, C25, C26, C27, C28, or larger alkyl chain. Thus, the hydrophobic tail can comprise a moiety having an optionally substituted C5 alkyl chain.

### 1. Nitric Oxide

Nitric oxide (NO) plays an essential role in biology, physiology, and pathophysiology, a key signaling molecule in vessel wall homeostasis. In addition, NO is an endothelium-derived relaxing factor produced in the vascular endothelial cells (VECs), which plays a crucial role in maintaining endothelial function and shows vasoprotective effects for the endothelium. NO production in VECs was achieved by oxidizing the L-arginine and oxygen, catalyzed by nitric oxide synthase. It is also reported that the abnormal alteration of endothelial function is strongly associated with the imbalance between endogenous vasodilator, NO, and other factors such as endothelium-dependent hyperpolarization, the enhanced oxidative stress state, and the generated vasoconstrictors. Moreover, NO also regulates vascular tone, lowers lipid levels, and inhibits adhesion molecule expressions, platelet aggregation, and vascular smooth muscle cell proliferation. In particular, NO released from the endothelium leads to increased concentrations of cyclic guanosine monophosphate (GMP) and cyclic GMP levels in SMCs, thereby resulting in the local relaxation of SMCs is essential for maintaining laminar blood flow for the blood vessel. In addition to SMCs, cyclic GMP in platelet was also increased by NO and suppressed the activation and adhesion of platelets to the endothelium. NO is also known for promoting endothelial cell growth, survival, and migration. Thus, insufficient NO bioavailability in the cardiovascular system may lead to endothelial injury and atherosclerosis development. Meanwhile, NO is a potent mediator of inflammation and immune response, shown to have anti-inflammatory effects. Despite NO's excellent properties, it is challenging to handle NO due to the necessity of complete oxygen exclusion. In addition, the use of NO in the clinic is still limited due to its high reactivity and short diffusion distance. Therefore, the delivery of NO by carriers has been investigated.

Several groups have synthesized exogenous NO donors, such as S-nitrosothiols (RSNOs), n-diazeniumdiolates (NONOates), and nitrosamines, which can generate NO under specific conditions. However, these exogenous NO donors are commonly administrated by systematic delivery, which suffers from short half-lives and ineffective delivery. In addition, burst release may occur when the NO is released from these exogenous NO donors, possibly resulting in adverse local toxicity. Therefore, drug delivery systems have been developed to solve those problems, including nanomaterials such as liposomes, micelles, dendrimers, peptide amphiphiles, inorganic nanoparticles, carbon nanotubes, and polymer nanoparticles. For instance, nontoxic polyamide dendrimers bearing 18 NO-releasing groups showed potent anti-inflammatory activity, as demonstrated by a significant inhibition of IL-8 production. In addition, PAMAM dendrimers were reported to inhibit thrombin-mediated platelet aggregation.

The disclosed NO-associated compound herein from peptide amphiphile 4, and 2 is NONOates. The chemical structure of this compound can be inferred from the name: diazen N═N, ium: formal positive charge, diolate: two negative oxygens.

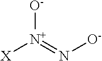

Diazeniumdiolates can be formed by the reaction of a nucleophilic amine (X—) with NO as shown in the following reaction:

X⁻+2NO→X—[N(O)NO]⁻

Diazoniumdiolates then dissociate on protonation to release free NO as shown in the following reaction:

X—[N(O)NO]⁻→X⁻+2NO

Notably, as shown below, the peptides amphiphile 4, amphiphile 6, and 8 for making the disclosed composition for improving the treatment of cardiovascular diseases such as atherosclerosis have nucleophilic amine on their side chain of the amino acid (lysine). The pendant amine groups of the lysine of the peptide react with NO to form a diazeniumdiolate-modified peptide [K(NO)NO⁻]]ₙ, wherein “n” is from 1 to 20. As the nitric oxide producing donor sequence of both peptide amphiphile 4 and amphiphile 2 comprise the amino acid sequence Lys-Lys-Lys-Lys-Lys, one or more lysine residues comprising the pendant amine group can react with NO to form a diazeniumdiolate-modified peptide [K(NO)NO⁻]]ₙ, where n can be 1, 2, 3, 4, or 5.

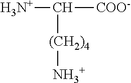

Notably, some earlier studies showed that NO released from polymers possessing lysine could suppress platelet attachment and SMC proliferation, contributing significantly to restenosis.

### 2. Hydrophilic Peptide Sequences and Hydrophobic Tail

In the disclosed invention, the peptide amphiphiles for making the composition can comprise one or more hydrophilic peptide sequences and a hydrophobic tail. There are three types of hydrophilic peptide sequences in the peptide amphiphiles that can be for making the disclosed compositions.

In an aspect, the first peptide sequence of the peptide amphiphiles can be a degradation sequence, comprising an amino acid sequence that undergoes cell-mediated proteolytic degradation. The degradation sequence can comprises an MMP2 specific cleavage site, wherein comprises an amino acid sequence Gly-Thr-Ala-Gly-Leu-Ile-Gly-Gln (GTAGLIGQ; SEQ ID NO:1). MMPs are zinc-dependent endopeptidases belonging to a larger family of proteases known as the metzincin Superfamily. The MMPs can be divided into four types, the collagenases, the gelatinases, the stromelysins, and the membrane-type MMPs (MT-MMPs), dependent on the MMPs' substrate specificity and intercellular location. In particular, the collagenases are responsible for degrading triple-helicalcollagens, the significant components of bone and cartilage, into distinctive fragments. The traditional collagenase family includes MMP1, MMP8, MMP13, and MMP18, while MMP2 and MM9 belong to gelatinases. The primary substrates of the gelatinases are type IV collagen and gelatin. In contrast to collagenase, the stromelysins can cleave extracellular matrix proteins but not the triple-helical fibrillar collagens, including MMP3, MMP10, and MMP11. In addition to the discussed MMPs, other MMPs, such as MMP14, MMP15, MMP16, MMP17, MMP24, and MMP25, are MT-MMPs.

In an aspect, endothelial cell adhesive sequences comprise the amino acid sequence of Tyr-Ile-Gly-Ser-Arg (YIGSR; SEQ ID NO:2) can be used. YIGSR is a synthetic laminin-derived pentapeptide. Laminins are a non-collagenous glycoprotein from basement membranes, essential for building a cellular network that connects the intracellular and extracellular components. YIGSR has been shown to improve cell adhesion, regulate myoblast cell function, and promote laminin receptor binding. Incorporating the YIGSR sequence in polyurethane has been shown to enhance endothelial cell adhesion and spreading but inhibit smooth muscle cell proliferation. In some aspects, peptide amphiphiles with YIGSR sequence can significantly improved endothelial cell adhesion, spreading, and proliferation while remarkably reducing platelet adhesion.

In an aspect, a nitric oxide-producing donor sequence comprising an amino acid sequence Lys-Lys-Lys-Lys-Lys (SEQ ID NO:3: KKKKK) can be used.

In some aspects, the hydrophobic tail of the peptide amphiphile can comprise a moiety having an optionally substituted C4 or larger alkyl chain. Thus, the hydrophobic tail can comprise a moiety having an optionally substituted C6 to C28 or a larger alkyl chain. Thus, the hydrophobic tail can comprise a moiety having an optionally substituted C10 to C25 or larger alkyl chain. Thus, the hydrophobic tail can comprise a moiety having an optionally substituted C4, C5, C6, C7, C8, C9, C10, C11, C12, C13, C14, C15, C16, C17, C18, C19, C20, C21, C22, C23, C24, C25, C26, C27, C28, or larger alkyl chain. Thus, the hydrophobic tail can comprise a moiety having an optionally substituted C5 or C16 alkyl chain.

### 3. Modified DS Peptide Amphiphiles

Disclosed are modified DS peptide amphiphiles, wherein a modified DS peptide amphiphile comprises at least one substitution in the degrading sequence (DS) compared to a wild type peptide amphiphile. In some aspects, a wild type peptide amphiphile can be any of the peptide amphiphiles described herein.

In an aspect, disclosed are one or more of the peptide amphiphiles described herein, wherein the hydrophilic peptide sequence comprises a degrading sequence (DS), wherein the DS comprises the amino acid sequence GTAGLIGQ (SEQ ID NO:1), wherein the DS comprises one or more amino acid substitutions to SEQ ID NO:1.

In some aspects, the hydrophobic tail of the disclosed peptide amphiphiles, comprise a substituted C5 alkyl chain and a degrading sequence. For example, disclosed herein is a peptide amphiphile, PA-05-GTAGLIGQ-YIGSR that comprises multiple hydrophobic amino acids, including glycine (G), alanine (A), leucine (L), and isoleucine (I), and tyrosine (Y). The hydrophobic portions can lead to a solubility issue of the peptide amphiphile in both aqueous and organic solvents and ununiform coating by using it for medical devices. Thus, to solve the issue, disclosed herein are peptide amphiphiles with significantly improved solubility. In some aspects, the peptide amphiphiles disclosed herein comprise a sequence wherein one or multiple amino acids of the sequence portion (GTAGLIGQ) are substituted with a Lysine (K). In some aspects, the original endothelial adhesive ligand sequence, YIGSR of a peptide amphpiphile can be part of the disclosed peptide amphiphiles. In some aspects, the disclosed peptide amphiphiles can be used with other peptide amphiphiles, such as PA-05-KKKKK-NO, as coating materials to significantly improve the technology based on peptide amphiphile PA-05-GTAGLIGQ-YIGSR.

Disclosed are peptide amphiphiles, wherein the peptide amphiphile comprises at least one substitution in a degrading sequence (DS) compared to DS sequence GTAGLIGQ. In some aspects, the peptide amphiphiles can be a modified version of any of the peptide amphiphiles described herein. Thus, in some aspects, the peptide amphiphiles having at least one substitution in a degrading sequence (DS) can be referred to as modified peptide amphiphiles.

Disclosed are peptide amphiphiles comprising a hydrophilic peptide sequence and a hydrophobic tail, wherein the hydrophobic tail comprises a moiety having a substituted C5 alkyl chain, wherein the hydrophilic peptide sequence comprises a degrading sequence (DS), wherein the degrading sequence (DS) comprises the amino acid sequence GTAGLIGQ (SEQ ID NO:1) wherein the DS comprises one or more amino acid substitutions. In some aspects, the one or more amino acid substitutions of SEQ ID NO: 1 can be any amino acid substitution. In some aspects, the amino acid substitution of SEQ ID NO: 1 can be a negatively charged amino acid or a polar uncharged amino acid. In some aspects, the amino acid substitution of SEQ ID NO: 1 can be arginine (Arg) and histidine (His), aspartic acid (Asp) and glutamic acid (Glu), serine (Ser), threonine (Thr), asparagine (Asn) and glutamine (Gln), and cysteine (Cys).

In some aspects, the DS comprises the sequence, GTAGLIGK, GTAGLIKK, GTAGLKKK, GTAGKKKK, GTAKKKKK, GTKKKKKK, GKKKKKKK or KKKKKKKK.

In some aspects, the one or more substituted amino acids can be on the N-terminal end, C-terminal end, or in the middle of the DS.

In some aspects, the disclosed peptide amphiphiles having a DS with one or more substitutions comprise a hydrophilic peptide sequence and a hydrophobic tail, wherein the hydrophobic tail comprises a moiety having a substituted C5 alkyl chain, wherein the hydrophilic peptide sequence comprises a degrading sequence (DS) wherein the degrading sequence (DS) comprises an amino acid sequence comprising the sequence X₁X₂X₃X₄X₅X₆X₇X₈, wherein any of X1 -X8 can be any amino acid. In some aspects, the DS comprises the sequence, GTAGLIGK, GTAGLIKK, GTAGLKKK, GTAGKKKK, GTAKKKKK, GTKKKKKK, GKKKKKKK or KKKKKKKK.

In some aspects, the disclosed peptide amphiphiles having a DS with one or more substitutions comprise a hydrophilic peptide sequence and a hydrophobic tail, wherein the hydrophobic tail comprises a moiety having a substituted C5 alkyl chain, wherein the hydrophilic peptide sequence comprises a degrading sequence (DS) wherein the degrading sequence (DS) comprises an amino acid sequence comprising the sequence X₁X₂X₃X₄X₅X₆X₇X₈, wherein X1 is Gly, a positive amino acid, a negatively charged amino acid or a polar uncharged amino acid; wherein X2 is Thr, a positive amino acid, a negatively charged amino acid or a polar uncharged amino acid; wherein X3 is Ala, a positive amino acid, a negatively charged amino acid or a polar uncharged amino acid; wherein X4 is r Gly, a positive amino acid, a negatively charged amino acid or a polar uncharged amino acid; wherein X5 is Leu, a positive amino acid, a negatively charged amino acid or a polar uncharged amino acid; wherein X6 is Ile, a positive amino acid, a negatively charged amino acid or a polar uncharged amino acid; wherein X7 is Gly, a positive amino acid, a negatively charged amino acid or a polar uncharged amino acid; and wherein X8 is Gln. a positive amino acid, a negatively charged amino acid or a polar uncharged amino acid. In some aspects, the DS comprises the sequence, GTAGLIGK, GTAGLIKK, GTAGLKKK, GTAGKKKK, GTAKKKKK, GTKKKKKK, GKKKKKKK or KKKKKKKK.

In some aspects, disclosed herein are peptide amphiphiles comprising a hydrophilic peptide sequence and a hydrophobic tail, wherein the hydrophobic tail comprises a moiety having a substituted C5 alkyl chain, wherein the hydrophilic peptide sequence comprises a degrading sequence (DS) wherein the degrading sequence (DS) comprises an amino acid sequence comprising the sequence (X₁X₂X₃X₄X₅X₆X₇X₈), wherein X1 is Lys or Gly, X2 is Lys or Thr, X3 is Lys or Ala, X4 is Lys or Gly, X5 is Lys or Leu, X6 is Lys or Ile, X7 is Lys or Gly, and X8 is Lys or Gln. In some aspects, the DS comprises the sequence, GTAGLIGK, GTAGLIKK, GTAGLKKK, GTAGKKKK, GTAKKKKK, GTKKKKKK, GKKKKKKK or KKKKKKKK.

In some aspects, all but one, all but two, all but three, all but four, all but five, all but six, or all but seven of the amino acids of the DS (e.g. SEQ ID NO: 1 or X₁X₂X₃X₄X₅X₆X₇X₈) are substituted.

In some aspects, the DS of the disclosed peptide amphiphiles comprises a MMP specific cleavage site.

In some aspects, the disclosed peptide amphiphiles further comprise a cell-adhesive sequence. In some aspects, the cell-adhesive sequence is an endothelial cell adhesive sequence that does not bind to smooth muscle cells and/or platelets. In some aspects, the cell-adhesive sequence comprises the amino acid sequence YIGSR (SEQ ID NO:2).

### C. Compositions

Disclosed are compositions comprising one or more of the peptide amphiphiles described herein. For example, disclosed herein are compositions comprising a peptide amphiphile comprising a hydrophilic peptide sequence and a hydrophobic tail, wherein the hydrophobic tail comprises a moiety having a substituted C5 alkyl chain, wherein the hydrophilic peptide sequence comprises a nitric oxide producing donor sequence.

Disclosed herein are compositions comprising peptide amphiphiles comprising a hydrophilic peptide sequence and a hydrophobic tail, wherein the hydrophobic tail comprises a moiety having a substituted C5 alkyl chain, wherein the hydrophilic peptide sequence comprises a nitric oxide producing donor sequence wherein the nitric oxide producing donor sequence comprises the amino acid sequence Lys-Lys-Lys-Lys-Lys (KKKKK) (K5; SEQ ID NO:3).

Disclosed herein are compositions comprising peptide amphiphile comprising a hydrophilic peptide sequence and a hydrophobic tail, wherein the hydrophobic tail comprises a moiety having a substituted C5 alkyl chain, wherein the hydrophilic peptide sequence comprises a nitric oxide producing donor sequence wherein the nitric oxide producing donor sequence comprises the amino acid sequence Lys-Lys-Lys-Lys-Lys (KKKKK) (K5; SEQ ID NO:3) and wherein one or more of the Lys residues comprise a pendant amine group.

Disclosed herein are compositions comprising peptide amphiphiles comprising a hydrophilic peptide sequence and a hydrophobic tail, wherein the hydrophobic tail comprises a moiety having a substituted C16 alkyl chain, wherein the hydrophilic peptide sequence comprises a nitric oxide producing donor sequence comprising an amino acid sequence Lys-Lys-Lys-Lys-Lys (KKKKK; SEQ ID NO:3) and a degrading sequence (DS) comprising an amino acid sequence Gly-Thr-Ala-Gly-Leu-ILE-Gly-Gln (GTAGLIGQ; SEQ ID NO:1), wherein one or more of the lysine residues comprise a pendant amine group. In some aspects, the peptide amphiphiles comprise one or more molecules of nitric oxide bound to at least one pendant amine group. Disclosed herein are compositions comprising peptide Amphiphiles 9, 10, or 11.

Disclosed herein are compositions comprising mixtures of peptide amphiphiles. In an aspect, disclosed herein are compositions comprising a mixture of two peptide amphiphiles, wherein the first peptide amphiphile comprises a hydrophilic peptide sequence and a hydrophobic tail, wherein the hydrophobic tail comprises a moiety having a substituted C16 alkyl chain, wherein the hydrophilic peptide sequence comprises an endothelial cell-adhesive sequence (CA) comprising an amino acid sequence Tyr-Ile-Gly-Ser-Arg (YIGSR; SEQ ID NO:2) and a degrading sequence (DS) comprising an amino acid sequence Gly-Thr-Ala-Gly-Leu-ILE-Gly-Gln (GTAGLIGQ; SEQ ID NO:1), and wherein the second peptide amphiphile comprises comprises a hydrophilic peptide sequence and a hydrophobic tail, wherein the hydrophobic tail comprises a moiety having optionally substituted C16 alkyl chain, wherein the hydrophilic peptide sequence comprises a nitric oxide producing donor sequence comprising an amino acid sequence Lys-Lys-Lys-Lys-Lys (KKKKK) (K5; SEQ ID NO:3) and also comprises a degrading sequence (DS) comprising an amino acid sequence Gly-Thr-Ala-Gly-Leu-ILE-Gly-Gln (GTAGLIGQ; SEQ ID NO:1). In some aspects, the peptide amphiphiles comprise one or more molecules of nitric oxide bound to at least one pendant amine group. In some aspects, disclosed are compositions comprising a mixture of two or more of the peptide amphiphiles disclosed herein. For example, disclosed are compositions comprising a mixture of two or more peptide amphiphiles, wherein the two or more peptide amphiphiles are two or more of peptide amphiphiles 1-12. Also disclosed are compositions comprising a mixture of two or more peptide amphiphiles, wherein the two or more peptide amphiphiles are two or more of peptide amphiphiles 1-12 wherein one or more of the peptide amphiphiles further comprise nitric oxide.

Disclosed herein are compositions comprising peptide amphiphiles comprising a hydrophilic peptide sequence and a hydrophobic tail, wherein the hydrophobic tail comprises a moiety having a substituted C5 alkyl chain, wherein the hydrophilic peptide sequence comprises a nitric oxide producing donor sequence, comprising the amino acid sequence Lys-Lys-Lys-Lys-Lys (KKKKK; SEQ ID NO:3) and wherein one or more of the Lys residues comprise a pendant amine group. In some aspects, the peptide amphiphiles comprise one or more molecules of nitric oxide bound to at least one pendant amine group.

Disclosed are compositions comprising peptide amphiphiles comprising a hydrophilic peptide sequence and a hydrophobic tail, wherein the hydrophobic tail comprises a moiety having a substituted C5 alkyl chain, wherein the hydrophilic peptide sequence comprises a degrading sequence (DS), wherein the degrading sequence (DS) comprises the amino acid sequence GTAGLIGQ (SEQ ID NO:1) wherein the DS comprises one or more amino acid substitutions.

Disclosed are compositions comprising peptide amphiphiles comprising a hydrophilic peptide sequence and a hydrophobic tail, wherein the hydrophobic tail comprises a moiety having a substituted C5 alkyl chain, wherein the hydrophilic peptide sequence comprises a degrading sequence (DS) wherein the degrading sequence (DS) comprises an amino acid sequence comprising the sequence (X₁X₂X₃X₄X₅X₆X₇X₈), wherein X1 is Gly, a positive amino acid, a negatively charged amino acid or a polar uncharged amino acid; wherein X2 is Thr, a positive amino acid, a negatively charged amino acid or a polar uncharged amino acid; wherein X3 is Ala, a positive amino acid, a negatively charged amino acid or a polar uncharged amino acid; wherein X4 is r Gly, a positive amino acid, a negatively charged amino acid or a polar uncharged amino acid; wherein X5 is Leu, a positive amino acid, a negatively charged amino acid or a polar uncharged amino acid; wherein X6 is Ile, a positive amino acid, a negatively charged amino acid or a polar uncharged amino acid; wherein X7 is Gly, a positive amino acid, a negatively charged amino acid or a polar uncharged amino acid; and wherein X8 is Gln. a positive amino acid, a negatively charged amino acid or a polar uncharged amino acid.

In some aspects, the composition is a pharmaceutical composition. In some aspects, the disclosed compositions comprise one or more of the peptide amphiphiles and a pharmaceutically acceptable carrier.

In some aspects, the pharmaceutical compositions described herein can be sterile and contain any of the disclosed compositions for producing the desired response in a unit of weight or volume suitable for administration to a subject. In some aspects, the pharmaceutical compositions can contain suitable buffering agents, including, e.g., acetic acid in a salt; citric acid in a salt; boric acid in a salt; and phosphoric acid in a salt.

When administered, the disclosed compositions or pharmaceutical compositions can be administered in pharmaceutically acceptable preparations. Such preparations may routinely contain pharmaceutically acceptable concentrations of salt, buffering agents, preservatives, compatible carriers, supplementary immune potentiating agents such as adjuvants and cytokines, and optionally other therapeutic agents.

As used herein, the term “pharmaceutically acceptable” means a non-toxic material that does not interfere with the effectiveness of the biological activity of the active ingredients. The term “physiologically acceptable” refers to a non-toxic material that is compatible with a biological system such as a cell, cell culture, tissue, or organism. The characteristics of the carrier will depend on the route of administration. Physiologically and pharmaceutically acceptable carriers include diluents, fillers, salts, buffers, stabilizers, solubilizers, and other materials which are well known in the art. The term denotes an organic or inorganic ingredient, natural or synthetic, with which the active ingredient is combined to facilitate the application. The components of the pharmaceutical compositions also are capable of being co-mingled with the disclosed compositions, and with each other, in a manner such that there is no interaction which would substantially impair the desired pharmaceutical efficacy.

As used herein, the term “pharmaceutically acceptable carrier” refers to solvents, dispersion media, coatings, antibacterial, isotonic and absorption delaying agents, buffers, excipients, binders, lubricants, gels, surfactants that can be used as media for a pharmaceutically acceptable substance. The pharmaceutically acceptable carriers can be lipid-based or a polymer-based colloid. Examples of colloids include liposomes, hydrogels, microparticles, nanoparticles and micelles. The compositions can be formulated for administration by any of a variety of routes of administration, and can include one or more physiologically acceptable excipients, which can vary depending on the route of administration. Any of the compositions described herein can be administered in the form of a pharmaceutical composition.

As used herein, the term “excipient” means any compound or substance, including those that can also be referred to as “carriers” or “diluents.” Preparing pharmaceutical and physiologically acceptable compositions is considered routine in the art, and thus, one of ordinary skill in the art can consult numerous authorities for guidance if needed. The compositions can also include additional agents (e.g., preservatives).

The pharmaceutical compositions disclosed herein can be sterile and sterilized by conventional sterilization techniques developed for non-soluble biological material like collagens. Aqueous solutions can be packaged for use as is, or lyophilized, the lyophilized preparation, which is encompassed by the present disclosure, can be combined with a sterile aqueous carrier prior to administration. The pH of the pharmaceutical compositions typically will be between 3 and 11 (e.g., between about 5 and 9) or between 6 and 8 (e.g., between about 7 and 8). The resulting compositions in solid form can be packaged in multiple single dose units, each containing a fixed amount of the above-mentioned agent or agents, such as in a sealed package of tablets or capsules. The composition in solid form can also be packaged in a container for a flexible quantity, such as in a squeezable tube designed for a topically applicable cream or ointment. The compositions can also be formulated as powders, elixirs, suspensions, emulsions, solutions, syrups, aerosols, lotions, creams, ointments, gels, suppositories, sterile injectable solutions and sterile packaged powders. The active ingredient can be any of the adipose or devitalized adipose tissues described herein in combination with one or more pharmaceutically acceptable carriers.

In some aspects, administration of disclosed compositions or pharmaceutical compositions disclosed herein can be administered to mammals other than humans, e.g., for testing purposes or veterinary therapeutic purposes, can be carried out under substantially the same conditions as described above.

Disclosed are one or more of the peptide amphiphiles described herein and a pharmaceutically active agent. In some aspects, the pharmaceutically active agent is a therapeutic drug for cardiovascular disease or an antibiotic.

### 1. Peptide Amphiphile Plus Therapeutic

Disclosed are compositions comprising one or more peptide amphiphiles described herein or described in US Patent Application Publication No 2010/0119573, which is incorporated by reference in its entirety herein for its teaching of peptide amphiphiles, and a pharmaceutically active agent. Disclosed are compositions comprising one or more peptide amphiphiles described herein or described in US Patent Application Publication No 2010/0119573, which is incorporated by reference in its entirety herein for its teaching of peptide amphiphiles, and a pharmaceutically active agent, wherein the peptide amphiphile further comprises nitric oxide. In some aspects, the pharmaceutically active agent is a therapeutic drug or a therapeutic drug releasing liposome for cardiovascular disease.

Disclosed herein are compositions comprising one or more peptide amphiphiles and a pharmaceutically active agent, wherein the pharmaceutically active agent is a therapeutic drug or a therapeutic drug releasing liposome for cardiovascular disease. In some aspects, the therapeutic drug is sirolimus, everolimus, paclitaxel, colchicine, or a statin.

In some aspects, the therapeutic drug releasing liposome can be one or more of the therapeutic drug releasing liposomes described herein. In some aspects, a therapeutic drug is encapsulated in a liposome forming the therapeutic drug releasing liposome. For example, a therapeutic drug releasing liposome can comprise, or encapsulate, one or more of the peptide amphiphiles or compositions described throughout and a therapeutic drug.

Disclosed herein are compositions and peptide amphiphiles that further comprise colchicine. The chemical name of the colchicine is N[5′,6,7,9-tetrahydro-1,2,3,10-tetratmethoxy 9-oxobenzo[a]heptalen-7-yl], (S)-acetamide; molecular formula: C₂₂H₂₅NO₆; CAS number: 64-86-8. The chemical structure of colchicine is shown as follows:

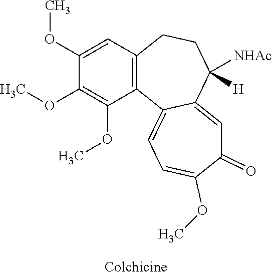 [SMILES: COc1cc2c(c(OC)c1OC)-c1ccc(OC)c(=O)cc1[C@@H](NC(C)=O)CC2]

Colchicine is a natural product used as an anti-inflammatory drug widely for treating symptomatic inflammatory diseases, such as gout. Colchicine is commonly extracted from two plants of the lily family, Colchicum autumnale and Gloriosa superba. Colchicine is a tricyclic alkaloid and has a molecular mass of 399.437. The active ingredient colchicine and its tablet formulation are listed in various national and international pharmacopeias such as the United States Pharmacopeia (USP).

Recently, colchicine has been studied for treating cardiovascular disease. For instance, In the Colchicine Cardiovascular Outcomes Trial (COLCOT) involving patients who had a myocardial infarction within 30 days before enrollment, the percentage of those who had the composite endpoint of cardiovascular death, resuscitated cardiac arrest, myocardial infarction, stroke, or urgent hospitalization for angina leading to coronary revascularization was lower among those who received 0.5 mg of colchicine once daily than among those who received placebo.

In an earlier trial of low-dose colchicine (LoDoCo) involving patients with chronic coronary disease, we found that the risk of acute cardiovascular events was lower among those who received 0.5 mg of colchicine once daily than those who did not receive colchicine. This was an open-label trial involving only 532 patients, and the results required confirmation. Accordingly, we conducted an investigator-initiated, randomized, controlled, double-blind, event-driven trial of low-dose colchicine (LoDoCo2) to determine whether 0.5 mg of colchicine once daily, as compared with placebo, prevents cardiovascular events in patients with chronic coronary disease.

Disclosed herein are compositions and peptide amphiphiles that further comprise sirolimus. Sirolimus is a white crystalline solid, of which the melting range is between 183° to 185° C. It is a lipophilic macrocyclic lactone structurally, soluble in most organic solvents, and virtually insoluble in water. Sirolimus is also known as rapamycin, the USAN-assigned generic name. Sirolimus is produced by a bacterium strain of Streptomyces hygroscopicus, known for its immunosuppressive and antiproliferative properties. Sirolimus consists of a 29-membered ring containing 4 trans double bonds, three of which are conjugated. The chemical structure of sirolimus is shown as follows:

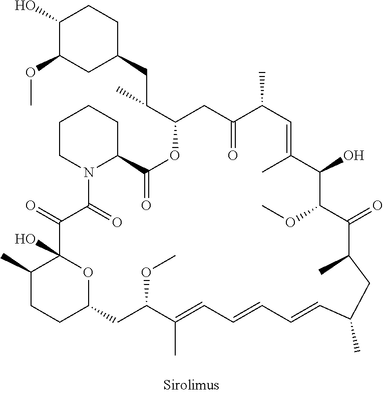 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)CC(=O)[C@H](C)/C=C(\C)[C@@H](O)[C@@H](OC)C(=O)[C@H](C)C[C@H](C)/C=C/C=C/C=C/1C]

Sirolimus is a potent inhibitor of S6K1 activation (a serine/threonine kinase) and a mediator of phosphoinositide 3-kinase (PI3K) signaling, which can form a gain-of-function complex with the FK506-binding protein (FKBP12) to bind and act as a specific allosteric inhibitor of the mechanistic target of rapamycin (mTOR) complex 1 (mTORC1). Because S6K and a eukaryotic translation initiation factor 4E (eIF4E)-binding protein 1 (4EBP1) are the main targets of mTORC1, mTOR inhibits 4EBP1 and activates S6K, thereby activating protein synthesis, ribosome biogenesis, nutrient transport, and lipid synthesis in response to nutrients, growth factors, and cellular energy. In addition, sirolimus can inhibit the activation of T and B cells.

Disclosed herein are compositions and peptide amphiphiles that further comprise a statin. Overwhelming evidence indicates that cardiovascular morbidity and mortality can be mitigated if the low-density lipoprotein cholesterol (LDL-C) is lowered. The frequently prescribed classic drugs for lowering cholesterol are statins, which are for primary and secondary prevention of CVD. The active part of statins is their modified 3,5-dihydroxyglutaric acid moiety, which is structurally similar to the endogenous substrate (hydroxymethylglutaryl-coenzyme A) HMG-CoA, and the mevaldyl CoA transition state intermediate, thus, statins can inhibit HMG-CoA reductase that can limit the rate of the cholesterol biosynthesis pathway. Commercially available statin drugs include atorvastatin, cerivastatin, fluvastatin, lovastatin, pitavastatin, pravastatin, rosuvastatin, and simvastatin. The structural differences of these statins are the rings attached to the active moiety. For instance, lovastatin, simvastatin, and pravastatin have a partially reduced naphthalene, while atorvastatin, Fluvastatin, and rosuvastatin have a pyrrole, an indole, and a pyrimidine, respectively. Below is the chemical structure of lovastatin.

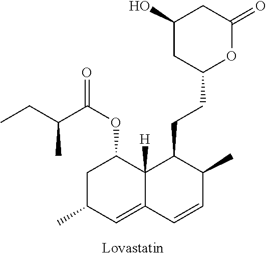 [SMILES: CC[C@H](C)C(=O)O[C@H]1C[C@@H](C)C=C2C=C[C@H](C)[C@H](CC[C@@H]3C[C@@H](O)CC(=O)O3)[C@H]21]

Statin therapy has demonstrated significant efficacy in lowering LDL-C levels. For instance, Statins only can decrease the 20-50% of the LDL-C level. In addition, it was reported that the LDL-C levels could be reduced to a low level (<40 mg/dL) when statin and PCSK9 inhibitors were used. Other Beneficial effects could include improved vascular endothelial function, cardiac regeneration, and re-endothelialization. Although statins provide lipid-lowering effects and reduce cardiovascular risk when administered systemically, they are often associated with systemic side effects, including muscle soreness, myopathy, liver damage, and increased risk of diabetes development. In addition, the more severe adverse effect may be associated with new-onset type 2, neurological and neurocognitive effects, hepatotoxicity, and renal toxicity. Local delivery of statins might provide greater stain concentration in target tissues while minimizing side effects.

Disclosed herein are compositions and peptide amphiphiles that further comprise one or more microRNAs (MiRNAs). In some aspects, one or more of the miRNAs provided in Tables 1-3 can be used in the disclosed compositions (SHOCK, 46(2), 122-131, (2016)).

TABLE 1
Examples of miRNAs that can regulate M1 and M2 polarization
through targeting various adaptor proteins and transcription
factors that can be used in the disclosed compositions.

| MiRNAs | Phenotype | Targets | Function |
|---|---|---|---|
| miR-124 | M2 | STAT3 | TACE Inhibits production of pro- |
| | | | inflammatory cytokines |
| mrR-223 | M2 | STAT3 | Promotes anti-inflammatory response |
| miR-34a | M2 | Nothc1 | Inhibits production of pro-inflammatory |
| | | | cytokines |
| Let-7c | M2 | PAK1 | Inhibits activation of NF-kB pathway |
| miR-132 | M2 | AChE | Promotes cholinergic anti-inflammatory |
| | | | response |
| miR-146a | M2 | IRAK1, | Prevents activation of NF-kB |
| | | TRAF6 | |
| miR-125a | M2 | KLF4 | Promotes M2 polarization |

*PAK1 indicates p21-activated kinase 1.

TABLE 2
Examples of bone regeneration related miRNAs
that can be used in the disclosed compositions.
(Current Genomics, 16, 441-452 (2015)).

| MicroRNA | Cell | Target Gene |
|---|---|---|
| Let-7f | Human MSC | Axin2 |
| miRNA-15b | Human MSC | BMPR2 |
| miR-20a | Human MSC | PPARg, Bambi, Crim1 |
| miR-21 | Human MSC | Spry1 |
| miR-30c | Human MSC | CAMTA1*, CXCL12*, |
| | | ITGB1*, FLT1* |
| miR-96 | Human MSC | FABP4 |
| miR-130b | Human MSC | CAMTA1*, CD44*, GDF6*, |
| | | PDGFRA*, COL9A3* |
| miR-199a | Human MSC | SOX9 |

*putative target gene

TABLE 3
Examples of vascular related miRNAs that can be used in the disclosed
compositions. (Cardiovascular Research, 110, 6-22, (2016)).

| miRNA | Modulation | Effect |
|---|---|---|
| miR-122 | Antagomir | Cholesterol decrease |
| miR-145 | Lentiviral overexpression | Plaque size and fibrosis decrease |
| miR-21 | Antagomir | Neointimal proliferation decrease |
| miR-126 | Apoptotic bodies | Lesion size decrease, vascular |
| | | repair increase |

In some aspects, compositions comprising one or more peptide amphiphiles and a therapeutic drug or a therapeutic drug releasing liposome comprise at least one peptide amphiphile comprising a) PA-C16-K5-NO; a hydrophilic peptide sequence and a hydrophobic tail, wherein the hydrophobic tail comprises a moiety having a substituted C16 alkyl chain, wherein the hydrophilic peptide sequence comprises a nitric oxide producing donor sequence comprising an amino acid sequence Lys-Lys-Lys-Lys-Lys (KKKKK; SEQ ID NO:3) and a degrading sequence (DS) comprising an amino acid sequence Gly-Thr-Ala-Gly-Leu-Ile-Gly-Gln (GTAGLIGQ; SEQ ID NO:1), wherein one or more of the lysine residues comprise a pendant amine group, wherein one or more molecules of NO is bound to at least one pendant amine group; b) PA-C16-YK5-NO; a mixture of two peptide amphiphiles, wherein the first peptide amphiphile comprises a hydrophilic peptide sequence and a hydrophobic tail, wherein the hydrophobic tail comprises a moiety having a substituted C16 alkyl chain, wherein the hydrophilic peptide sequence comprises an endothelial cell-adhesive sequence (CA) comprising an amino acid sequence Tyr-Ile-Gly-Ser-Arg (YIGSR; SEQ ID NO:2) and a degrading sequence (DS) comprising an amino acid sequence Gly-Thr-Ala-Gly-Leu-ILE-Gly-Gln (GTAGLIGQ; SEQ ID NO:1), (PA-C16-CA); wherein the second peptide amphiphile comprises comprises a hydrophilic peptide sequence and a hydrophobic tail, wherein the hydrophobic tail comprises a moiety having optionally substituted C16 alkyl chain, wherein the hydrophilic peptide sequence comprises a nitric oxide producing donor sequence comprising an amino acid sequence Lys-Lys-Lys-Lys-Lys (KKKKK) (1(5; SEQ ID NO:3) and also comprises a degrading sequence (DS) comprising an amino acid sequence Gly-Thr-Ala-Gly-Leu-ILE-Gly-Gln (GTAGLIGQ; SEQ ID NO:1), (PA-C16-K5), wherein one or more of the Lys residues of the second peptide amphiphile comprises a pendant amine group, wherein one or more molecules of NO is bound to at least one pendant amine group; c) PA-CS-KS-NO; a hydrophilic peptide sequence and a hydrophobic tail, wherein the hydrophobic tail comprises a moiety having a substituted C5 alkyl chain, wherein the hydrophilic peptide sequence comprises a nitric oxide producing donor sequence, comprising the amino acid sequence Lys-Lys-Lys-Lys-Lys (KKKKK; SEQ ID NO:3) and wherein one or more of the Lys residues comprise a pendant amine group, wherein one or more molecules of NO is bound to at least one of the pendant amine groups; or d) PA-C16-K5-YIGSR-NO; a hydrophilic peptide sequence and a hydrophobic tail, wherein the hydrophobic tail comprises a moiety having optionally substituted C16 alkyl chain, wherein the hydrophilic peptide sequence comprises a nitric oxide producing donor sequence comprising an amino acid sequence Lys-Lys-Lys-Lys-Lys (KKKKK) (1(.5; SEQ ID NO:3) and comprises a degrading sequence (DS) comprising an amino acid sequence Gly-Thr-Ala-Gly-Leu-ILE-Gly-Gln (GTAGLIGQ; SEQ ID NO:1), and also a cell-adhesive sequence comprising the amino acid sequence Tyr-Ile-Gly-Ser-Arg (YIGSR; SEQ ID NO:2) and further wherein one or more of the lysine residues comprise pendant amine groups that react with NO to form a diazeniumdiolate-modified peptide amphiphile.

Disclosed are compositions comprising one or more peptide amphiphiles and sirolimus or a sirolimus-releasing liposome, wherein the peptide amphiphiles comprise a) PA-C16-K5-NO; a hydrophilic peptide sequence and a hydrophobic tail, wherein the hydrophobic tail comprises a moiety having a substituted C16 alkyl chain, wherein the hydrophilic peptide sequence comprises a nitric oxide producing donor sequence comprising an amino acid sequence Lys-Lys-Lys-Lys-Lys (KKKKK; SEQ ID NO:3) and a degrading sequence (DS) comprising an amino acid sequence Gly-Thr-Ala-Gly-Leu-Ile-Gly-Gln (GTAGLIGQ; SEQ ID NO:1), wherein one or more of the lysine residues comprise a pendant amine group, wherein one or more molecules of NO is bound to at least one pendant amine group; b) PA-C16-YK5-NO; a mixture of two peptide amphiphiles, wherein the first peptide amphiphile comprises a hydrophilic peptide sequence and a hydrophobic tail, wherein the hydrophobic tail comprises a moiety having a substituted C16 alkyl chain, wherein the hydrophilic peptide sequence comprises an endothelial cell-adhesive sequence (CA) comprising an amino acid sequence Tyr-Ile-Gly-Ser-Arg (YIGSR; SEQ ID NO:2) and a degrading sequence (DS) comprising an amino acid sequence Gly-Thr-Ala-Gly-Leu-ILE-Gly-Gln (GTAGLIGQ; SEQ ID NO:1), (PA-C16-CA); wherein the second peptide amphiphile comprises comprises a hydrophilic peptide sequence and a hydrophobic tail, wherein the hydrophobic tail comprises a moiety having optionally substituted C16 alkyl chain, wherein the hydrophilic peptide sequence comprises a nitric oxide producing donor sequence comprising an amino acid sequence Lys-Lys-Lys-Lys-Lys (KKKKK) (K5; SEQ ID NO:3) and also comprises a degrading sequence (DS) comprising an amino acid sequence Gly-Thr-Ala-Gly-Leu-ILE-Gly-Gln (GTAGLIGQ; SEQ ID NO:1), (PA-C16-K5), wherein one or more of the Lys residues of the second peptide amphiphile comprises a pendant amine group, wherein one or more molecules of NO is bound to at least one pendant amine group; c) PA-05-K5-NO; a hydrophilic peptide sequence and a hydrophobic tail, wherein the hydrophobic tail comprises a moiety having a substituted C5 alkyl chain, wherein the hydrophilic peptide sequence comprises a nitric oxide producing donor sequence, comprising the amino acid sequence Lys-Lys-Lys-Lys-Lys (KKKKK; SEQ ID NO:3) and wherein one or more of the Lys residues comprise a pendant amine group, wherein one or more molecules of NO is bound to at least one of the pendant amine groups; or d) PA-C16-K5-YIGSR-NO; a hydrophilic peptide sequence and a hydrophobic tail, wherein the hydrophobic tail comprises a moiety having optionally substituted C16 alkyl chain, wherein the hydrophilic peptide sequence comprises a nitric oxide producing donor sequence comprising an amino acid sequence Lys-Lys-Lys-Lys-Lys (KKKKK) (1(5; SEQ ID NO:3) and comprises a degrading sequence (DS) comprising an amino acid sequence Gly-Thr-Ala-Gly-Leu-ILE-Gly-Gln (GTAGLIGQ; SEQ ID NO:1), and also a cell-adhesive sequence comprising the amino acid sequence Tyr-Ile-Gly-Ser-Arg (YIGSR; SEQ ID NO:2) and further wherein one or more of the lysine residues comprise pendant amine groups that react with NO to form a diazeniumdiolate-modified peptide amphiphile.

Disclosed are compositions comprising one or more peptide amphiphiles and everolimus or a everolimus releasing liposome, wherein the peptide amphiphiles comprise a) PA-C16-K5-NO; a hydrophilic peptide sequence and a hydrophobic tail, wherein the hydrophobic tail comprises a moiety having a substituted C16 alkyl chain, wherein the hydrophilic peptide sequence comprises a nitric oxide producing donor sequence comprising an amino acid sequence Lys-Lys-Lys-Lys-Lys (KKKKK; SEQ ID NO:3) and a degrading sequence (DS) comprising an amino acid sequence Gly-Thr-Ala-Gly-Leu-Ile-Gly-Gln (GTAGLIGQ; SEQ ID NO:1), wherein one or more of the lysine residues comprise a pendant amine group, wherein one or more molecules of NO is bound to at least one pendant amine group; b) PA-C16-YK5-NO; a mixture of two peptide amphiphiles, wherein the first peptide amphiphile comprises a hydrophilic peptide sequence and a hydrophobic tail, wherein the hydrophobic tail comprises a moiety having a substituted C16 alkyl chain, wherein the hydrophilic peptide sequence comprises an endothelial cell-adhesive sequence (CA) comprising an amino acid sequence Tyr-Ile-Gly-Ser-Arg (YIGSR; SEQ ID NO:2) and a degrading sequence (DS) comprising an amino acid sequence Gly-Thr-Ala-Gly-Leu-ILE-Gly-Gln (GTAGLIGQ; SEQ ID NO:1), (PA-C16-CA); wherein the second peptide amphiphile comprises a hydrophilic peptide sequence and a hydrophobic tail, wherein the hydrophobic tail comprises a moiety having optionally substituted C16 alkyl chain, wherein the hydrophilic peptide sequence comprises a nitric oxide producing donor sequence comprising an amino acid sequence Lys-Lys-Lys-Lys-Lys (KKKKK) (K5; SEQ ID NO:3) and also comprises a degrading sequence (DS) comprising an amino acid sequence Gly-Thr-Ala-Gly-Leu-ILE-Gly-Gln (GTAGLIGQ; SEQ ID NO:1), (PA-C16-K5), wherein one or more of the Lys residues of the second peptide amphiphile comprises a pendant amine group, wherein one or more molecules of NO is bound to at least one pendant amine group; c) PA-05-K5-NO; a hydrophilic peptide sequence and a hydrophobic tail, wherein the hydrophobic tail comprises a moiety having a substituted C5 alkyl chain, wherein the hydrophilic peptide sequence comprises a nitric oxide producing donor sequence, comprising the amino acid sequence Lys-Lys-Lys-Lys-Lys (KKKKK; SEQ ID NO:3) and wherein one or more of the Lys residues comprise a pendant amine group, wherein one or more molecules of NO is bound to at least one of the pendant amine groups; or d) PA-C16-K5-YIGSR-NO; a hydrophilic peptide sequence and a hydrophobic tail, wherein the hydrophobic tail comprises a moiety having optionally substituted C16 alkyl chain, wherein the hydrophilic peptide sequence comprises a nitric oxide producing donor sequence comprising an amino acid sequence Lys-Lys-Lys-Lys-Lys (KKKKK) (1(.5; SEQ ID NO:3) and comprises a degrading sequence (DS) comprising an amino acid sequence Gly-Thr-Ala-Gly-Leu-ILE-Gly-Gln (GTAGLIGQ; SEQ ID NO:1), and also a cell-adhesive sequence comprising the amino acid sequence Tyr-Ile-Gly-Ser-Arg (YIGSR; SEQ ID NO:2) and further wherein one or more of the lysine residues comprise pendant amine groups that react with NO to form a diazeniumdiolate-modified peptide amphiphile.

Disclosed are compositions comprising one or more peptide amphiphiles and colchicine or a colchicine-releasing liposome, wherein the peptide amphiphiles comprise a) PA-C16-K5-NO; a hydrophilic peptide sequence and a hydrophobic tail, wherein the hydrophobic tail comprises a moiety having a substituted C16 alkyl chain, wherein the hydrophilic peptide sequence comprises a nitric oxide producing donor sequence comprising an amino acid sequence Lys-Lys-Lys-Lys-Lys (KKKKK; SEQ ID NO:3) and a degrading sequence (DS) comprising an amino acid sequence Gly-Thr-Ala-Gly-Leu-Ile-Gly-Gln (GTAGLIGQ; SEQ ID NO:1), wherein one or more of the lysine residues comprise a pendant amine group, wherein one or more molecules of NO is bound to at least one pendant amine group; b) PA-C16-YK5-NO; a mixture of two peptide amphiphiles, wherein the first peptide amphiphile comprises a hydrophilic peptide sequence and a hydrophobic tail, wherein the hydrophobic tail comprises a moiety having a substituted C16 alkyl chain, wherein the hydrophilic peptide sequence comprises an endothelial cell-adhesive sequence (CA) comprising an amino acid sequence Tyr-Ile-Gly-Ser-Arg (YIGSR; SEQ ID NO:2) and a degrading sequence (DS) comprising an amino acid sequence Gly-Thr-Ala-Gly-Leu-ILE-Gly-Gln (GTAGLIGQ; SEQ ID NO:1), (PA-C16-CA); wherein the second peptide amphiphile comprises a hydrophilic peptide sequence and a hydrophobic tail, wherein the hydrophobic tail comprises a moiety having optionally substituted C16 alkyl chain, wherein the hydrophilic peptide sequence comprises a nitric oxide producing donor sequence comprising an amino acid sequence Lys-Lys-Lys-Lys-Lys (KKKKK) (K5; SEQ ID NO:3) and also comprises a degrading sequence (DS) comprising an amino acid sequence Gly-Thr-Ala-Gly-Leu-ILE-Gly-Gln (GTAGLIGQ; SEQ ID NO:1), (PA-C16-K5), wherein one or more of the Lys residues of the second peptide amphiphile comprises a pendant amine group, wherein one or more molecules of NO is bound to at least one pendant amine group; c) PA-05-K5-NO; a hydrophilic peptide sequence and a hydrophobic tail, wherein the hydrophobic tail comprises a moiety having a substituted C5 alkyl chain, wherein the hydrophilic peptide sequence comprises a nitric oxide producing donor sequence, comprising the amino acid sequence Lys-Lys-Lys-Lys-Lys (KKKKK; SEQ ID NO:3) and wherein one or more of the Lys residues comprise a pendant amine group, wherein one or more molecules of NO is bound to at least one of the pendant amine groups; or d) PA-C16-K5-YIGSR-NO; a hydrophilic peptide sequence and a hydrophobic tail, wherein the hydrophobic tail comprises a moiety having optionally substituted C16 alkyl chain, wherein the hydrophilic peptide sequence comprises a nitric oxide producing donor sequence comprising an amino acid sequence Lys-Lys-Lys-Lys-Lys (KKKKK) (1(5; SEQ ID NO:3) and comprises a degrading sequence (DS) comprising an amino acid sequence Gly-Thr-Ala-Gly-Leu-ILE-Gly-Gln (GTAGLIGQ; SEQ ID NO:1), and also a cell-adhesive sequence comprising the amino acid sequence Tyr-Ile-Gly-Ser-Arg (YIGSR; SEQ ID NO:2) and further wherein one or more of the lysine residues comprise pendant amine groups that react with NO to form a diazeniumdiolate-modified peptide amphiphile.

Disclosed are compositions comprising one or more peptide amphiphiles and a statin or a statin-releasing liposome, wherein the peptide amphiphiles comprise a) PA-C16-K5-NO; a hydrophilic peptide sequence and a hydrophobic tail, wherein the hydrophobic tail comprises a moiety having a substituted C16 alkyl chain, wherein the hydrophilic peptide sequence comprises a nitric oxide producing donor sequence comprising an amino acid sequence Lys-Lys-Lys-Lys-Lys (KKKKK; SEQ ID NO:3) and a degrading sequence (DS) comprising an amino acid sequence Gly-Thr-Ala-Gly-Leu-Ile-Gly-Gln (GTAGLIGQ; SEQ ID NO:1), wherein one or more of the lysine residues comprise a pendant amine group, wherein one or more molecules of NO is bound to at least one pendant amine group; b) PA-C16-YK5-NO; a mixture of two peptide amphiphiles, wherein the first peptide amphiphile comprises a hydrophilic peptide sequence and a hydrophobic tail, wherein the hydrophobic tail comprises a moiety having a substituted C16 alkyl chain, wherein the hydrophilic peptide sequence comprises an endothelial cell-adhesive sequence (CA) comprising an amino acid sequence Tyr-Ile-Gly-Ser-Arg (YIGSR; SEQ ID NO:2) and a degrading sequence (DS) comprising an amino acid sequence Gly-Thr-Ala-Gly-Leu-ILE-Gly-Gln (GTAGLIGQ; SEQ ID NO:1), (PA-C16-CA); wherein the second peptide amphiphile comprises comprises a hydrophilic peptide sequence and a hydrophobic tail, wherein the hydrophobic tail comprises a moiety having optionally substituted C16 alkyl chain, wherein the hydrophilic peptide sequence comprises a nitric oxide producing donor sequence comprising an amino acid sequence Lys-Lys-Lys-Lys-Lys (KKKKK) (K5; SEQ ID NO:3) and also comprises a degrading sequence (DS) comprising an amino acid sequence Gly-Thr-Ala-Gly-Leu-ILE-Gly-Gln (GTAGLIGQ; SEQ ID NO:1), (PA-C16-K5), wherein one or more of the Lys residues of the second peptide amphiphile comprises a pendant amine group, wherein one or more molecules of NO is bound to at least one pendant amine group; or c) PA-05-K5-NO; a hydrophilic peptide sequence and a hydrophobic tail, wherein the hydrophobic tail comprises a moiety having a substituted C5 alkyl chain, wherein the hydrophilic peptide sequence comprises a nitric oxide producing donor sequence, comprising the amino acid sequence Lys-Lys-Lys-Lys-Lys (KKKKK; SEQ ID NO:3) and wherein one or more of the Lys residues comprise a pendant amine group, wherein one or more molecules of NO is bound to at least one of the pendant amine groups; or d) PA-C16-K5-YIGSR-NO; a hydrophilic peptide sequence and a hydrophobic tail, wherein the hydrophobic tail comprises a moiety having optionally substituted C16 alkyl chain, wherein the hydrophilic peptide sequence comprises a nitric oxide producing donor sequence comprising an amino acid sequence Lys-Lys-Lys-Lys-Lys (KKKKK) (K5; SEQ ID NO:3) and comprises a degrading sequence (DS) comprising an amino acid sequence Gly-Thr-Ala-Gly-Leu-ILE-Gly-Gln (GTAGLIGQ; SEQ ID NO:1), and also a cell-adhesive sequence comprising the amino acid sequence Tyr-Ile-Gly-Ser-Arg (YIGSR; SEQ ID NO:2) and further wherein one or more of the lysine residues comprise pendant amine groups that react with NO to form a diazeniumdiolate-modified peptide amphiphile.

2. Peptide Amphiphile plus Antibiotic

Disclosed are compositions comprising one or more peptide amphiphiles described herein or described in US Patent Application Publication No 2010/0119573 for its teaching of peptide amphiphiles, which is incorporated by reference in its entirety herein and a pharmaceutically active agent. In some aspects, the pharmaceutically active agent is an antibiotic or an antibiotic releasing liposome for preventing or treating infection.

Disclosed herein are compositions comprising one or more peptide amphiphiles and a pharmaceutically active agent, wherein the pharmaceutically active agent is an antibiotic or an antibiotic releasing liposome for treating or preventing infection. In some aspects, the antibiotic is gentamicin, vancomycin, ciprofloxacin or metronidazole.

Gentamicin, an aminoglycoside antibiotic, has been used to treat several gram-negative bacteria for infections, such as bone infection, pelvic inflammatory disease, and urinary tract infections. Commonly, gentamicin has been administrated through parenteral routes, such as systemic, topical, and ophthalmic formulations, including intramuscular and intravenous administration. The highly susceptible microorganisms to gentamicin are members of the Enterobacteriaceae family, such as Escherichia coli, Klebsiella pneumoniae, Serratia spp. and Enterobacter spp.), Pseudomonas aeruginosa, and some strains of Neisseria, Moraxella, and Haemophilus genera. The gentamicin is only effective for aerobic bacteria, as it enters the bacteria membrane through an oxygen-dependent active transport. After gentamicin passes through the membrane and stays in the cytoplasm, it leads to the formation of truncated or nonfunctional proteins by disturbing mRNA translation. Therefore, the gentamicin dosage used in the patient depends on the patient's weight. Although gentamicin is widely used in clinics, it still can result in inner van ear and kidney problems.

In contrast to gentamicin, vancomycin, a glycopeptide antibacterial, has been developed to treat severe gram-positive infections involving methicillin (methicillin)-resistant S. aureus (MRSA). Although vancomycin was regarded for its excellent efficacy more than its toxicity, recently, it was found that several types of bacteria, such as enterococci and later in staphylococci, showed resistance to vancomycin. Vancomycin is used to treat skin infections and bone and joint infections. Below are the chemical structures of gentamicin and vancomycin.

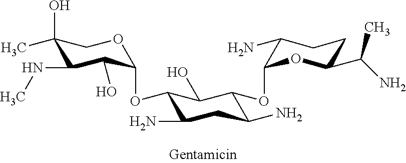

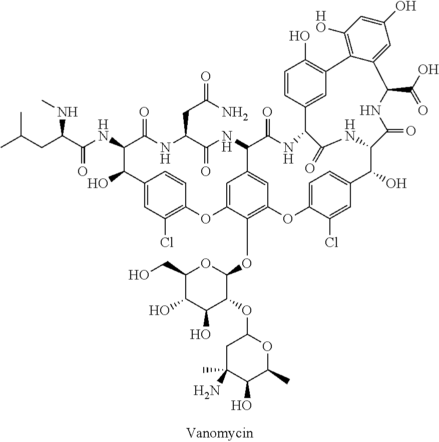

In some aspects, the antibiotic releasing liposome can be one or more of the antibiotic releasing liposomes described herein. In some aspects, an antibiotic is encapsulated in a liposome forming the antibiotic releasing liposome. For example, an antibiotic releasing liposome can comprise, or encapsulate, one or more of the peptide amphiphiles or compositions described throughout and an antibiotic.

In some aspects, compositions comprising one or more peptide amphiphiles and an antibiotic or antibiotic releasing liposome comprise at least one peptide amphiphile comprising a) PA-C16-K5-NO; a hydrophilic peptide sequence and a hydrophobic tail, wherein the hydrophobic tail comprises a moiety having a substituted C16 alkyl chain, wherein the hydrophilic peptide sequence comprises a nitric oxide producing donor sequence comprising an amino acid sequence Lys-Lys-Lys-Lys-Lys (KKKKK; SEQ ID NO:3) and a degrading sequence (DS) comprising an amino acid sequence Gly-Thr-Ala-Gly-Leu-Ile-Gly-Gln (GTAGLIGQ; SEQ ID NO:1), wherein one or more of the lysine residues comprise a pendant amine group, wherein one or more molecules of NO is bound to at least one pendant amine group; b) PA-C16-YK5-NO; a mixture of two peptide amphiphiles, wherein the first peptide amphiphile comprises a hydrophilic peptide sequence and a hydrophobic tail, wherein the hydrophobic tail comprises a moiety having a substituted C16 alkyl chain, wherein the hydrophilic peptide sequence comprises an endothelial cell-adhesive sequence (CA) comprising an amino acid sequence Tyr-Ile-Gly-Ser-Arg (YIGSR; SEQ ID NO:2) and a degrading sequence (DS) comprising an amino acid sequence Gly-Thr-Ala-Gly-Leu-ILE-Gly-Gln (GTAGLIGQ; SEQ ID NO:1), (PA-C16-CA); wherein the second peptide amphiphile comprises comprises a hydrophilic peptide sequence and a hydrophobic tail, wherein the hydrophobic tail comprises a moiety having optionally substituted C16 alkyl chain, wherein the hydrophilic peptide sequence comprises a nitric oxide producing donor sequence comprising an amino acid sequence Lys-Lys-Lys-Lys-Lys (KKKKK) (K5; SEQ ID NO:3) and also comprises a degrading sequence (DS) comprising an amino acid sequence Gly-Thr-Ala-Gly-Leu-ILE-Gly-Gln (GTAGLIGQ; SEQ ID NO:1), (PA-C16-K5), wherein one or more of the Lys residues of the second peptide amphiphile comprises a pendant amine group, wherein one or more molecules of NO is bound to at least one pendant amine group; c) PA-05-K5-NO; a hydrophilic peptide sequence and a hydrophobic tail, wherein the hydrophobic tail comprises a moiety having a substituted C5 alkyl chain, wherein the hydrophilic peptide sequence comprises a nitric oxide producing donor sequence, comprising the amino acid sequence Lys-Lys-Lys-Lys-Lys (KKKKK; SEQ ID NO:3) and wherein one or more of the Lys residues comprise a pendant amine group, wherein one or more molecules of NO is bound to at least one of the pendant amine groups; or PA-C16-K5-RGDS-NO; a hydrophilic peptide sequence and a hydrophobic tail, wherein the hydrophobic tail comprises a moiety having optionally substituted C16 alkyl chain, wherein the hydrophilic peptide sequence comprises a nitric oxide producing donor sequence comprising an amino acid sequence Lys-Lys-Lys-Lys-Lys (KKKKK) (1(.5; SEQ ID NO:3) and comprises a degrading sequence (DS) comprising an amino acid sequence Gly-Thr-Ala-Gly-Leu-ILE-Gly-Gln (GTAGLIGQ; SEQ ID NO:1), and further comprising a cell adhesive ligand comprising an amino acid sequence Arg-Gly-Asp-Ser (RGDS; SEQ ID NO:4), wherein herein one or more of the lysine residues comprise pendant amine groups that react with NO to form a diazeniumdiolate-modified peptide amphiphile; or PA-C16-K5-YIGSR-NO; a hydrophilic peptide sequence and a hydrophobic tail, wherein the hydrophobic tail comprises a moiety having optionally substituted C16 alkyl chain, wherein the hydrophilic peptide sequence comprises a nitric oxide producing donor sequence comprising an amino acid sequence Lys-Lys-Lys-Lys-Lys (KKKKK) (K5; SEQ ID NO:3) and comprises a degrading sequence (DS) comprising an amino acid sequence Gly-Thr-Ala-Gly-Leu-ILE-Gly-Gln (GTAGLIGQ; SEQ ID NO:1), and further comprising a cell adhesive ligand comprising an amino acid sequence Tyr-Ile-Gly-Ser-Arg (YIGSR; SEQ ID NO:2), wherein herein one or more of the lysine residues comprise pendant amine groups that react with NO to form a diazeniumdiolate-modified peptide amphiphile.

Disclosed are compositions comprising one or more peptide amphiphiles and gentamicin or a gentamicin-releasing liposome, wherein the peptide amphiphiles comprise a) PA-C16-K5-NO; a hydrophilic peptide sequence and a hydrophobic tail, wherein the hydrophobic tail comprises a moiety having a substituted C16 alkyl chain, wherein the hydrophilic peptide sequence comprises a nitric oxide producing donor sequence comprising an amino acid sequence Lys-Lys-Lys-Lys-Lys (KKKKK; SEQ ID NO:3) and a degrading sequence (DS) comprising an amino acid sequence Gly-Thr-Ala-Gly-Leu-Ile-Gly-Gln (GTAGLIGQ; SEQ ID NO:1), wherein one or more of the lysine residues comprise a pendant amine group, wherein one or more molecules of NO is bound to at least one pendant amine group; b) PA-C16-YK5-NO; a mixture of two peptide amphiphiles, wherein the first peptide amphiphile comprises a hydrophilic peptide sequence and a hydrophobic tail, wherein the hydrophobic tail comprises a moiety having a substituted C16 alkyl chain, wherein the hydrophilic peptide sequence comprises an endothelial cell-adhesive sequence (CA) comprising an amino acid sequence Tyr-Ile-Gly-Ser-Arg (YIGSR; SEQ ID NO:2) and a degrading sequence (DS) comprising an amino acid sequence Gly-Thr-Ala-Gly-Leu-ILE-Gly-Gln (GTAGLIGQ; SEQ ID NO:1), (PA-C16-CA); wherein the second peptide amphiphile comprises a hydrophilic peptide sequence and a hydrophobic tail, wherein the hydrophobic tail comprises a moiety having optionally substituted C16 alkyl chain, wherein the hydrophilic peptide sequence comprises a nitric oxide producing donor sequence comprising an amino acid sequence Lys-Lys-Lys-Lys-Lys (KKKKK) (K5; SEQ ID NO:3) and also comprises a degrading sequence (DS) comprising an amino acid sequence Gly-Thr-Ala-Gly-Leu-ILE-Gly-Gln (GTAGLIGQ; SEQ ID NO:1), (PA-C16-K5), wherein one or more of the Lys residues of the second peptide amphiphile comprises a pendant amine group, wherein one or more molecules of NO is bound to at least one pendant amine group; c) PA-05-K5-NO; a hydrophilic peptide sequence and a hydrophobic tail, wherein the hydrophobic tail comprises a moiety having a substituted C5 alkyl chain, wherein the hydrophilic peptide sequence comprises a nitric oxide producing donor sequence, comprising the amino acid sequence Lys-Lys-Lys-Lys-Lys (KKKKK; SEQ ID NO:3) and wherein one or more of the Lys residues comprise a pendant amine group, wherein one or more molecules of NO is bound to at least one of the pendant amine groups; PA-C16-K5-RGDS-NO; a hydrophilic peptide sequence and a hydrophobic tail, wherein the hydrophobic tail comprises a moiety having optionally substituted C16 alkyl chain, wherein the hydrophilic peptide sequence comprises a nitric oxide producing donor sequence comprising an amino acid sequence Lys-Lys-Lys-Lys-Lys (KKKKK) (K5; SEQ ID NO:3) and comprises a degrading sequence (DS) comprising an amino acid sequence Gly-Thr-Ala-Gly-Leu-ILE-Gly-Gln (GTAGLIGQ; SEQ ID NO:1), and further comprising a cell adhesive ligand comprising an amino acid sequence Arg-Gly-Asp-Ser (RGDS; SEQ ID NO:4), wherein herein one or more of the lysine residues comprise pendant amine groups that react with NO to form a diazeniumdiolate-modified peptide amphiphile; or PA-C16-K5-YIGSR-NO; a hydrophilic peptide sequence and a hydrophobic tail, wherein the hydrophobic tail comprises a moiety having optionally substituted C16 alkyl chain, wherein the hydrophilic peptide sequence comprises a nitric oxide producing donor sequence comprising an amino acid sequence Lys-Lys-Lys-Lys-Lys (KKKKK) (K5; SEQ ID NO:3) and comprises a degrading sequence (DS) comprising an amino acid sequence Gly-Thr-Ala-Gly-Leu-ILE-Gly-Gln (GTAGLIGQ; SEQ ID NO:1), and further comprising a cell adhesive ligand comprising an amino acid sequence Tyr-Ile-Gly-Ser-Arg (YIGSR; SEQ ID NO:2), wherein herein one or more of the lysine residues comprise pendant amine groups that react with NO to form a diazeniumdiolate-modified peptide amphiphile.

Disclosed are compositions comprising one or more peptide amphiphiles and vancomycin or a vancomycin-releasing liposome, wherein the peptide amphiphiles comprise a) PA-C16-1(5-NO; a hydrophilic peptide sequence and a hydrophobic tail, wherein the hydrophobic tail comprises a moiety having a substituted C16 alkyl chain, wherein the hydrophilic peptide sequence comprises a nitric oxide producing donor sequence comprising an amino acid sequence Lys-Lys-Lys-Lys-Lys (KKKKK; SEQ ID NO:3) and a degrading sequence (DS) comprising an amino acid sequence Gly-Thr-Ala-Gly-Leu-Ile-Gly-Gln (GTAGLIGQ; SEQ ID NO:1), wherein one or more of the lysine residues comprise a pendant amine group, wherein one or more molecules of NO is bound to at least one pendant amine group; b) PA-C16-YK5-NO; a mixture of two peptide amphiphiles, wherein the first peptide amphiphile comprises a hydrophilic peptide sequence and a hydrophobic tail, wherein the hydrophobic tail comprises a moiety having a substituted C16 alkyl chain, wherein the hydrophilic peptide sequence comprises an endothelial cell-adhesive sequence (CA) comprising an amino acid sequence Tyr-Ile-Gly-Ser-Arg (YIGSR; SEQ ID NO:2) and a degrading sequence (DS) comprising an amino acid sequence Gly-Thr-Ala-Gly-Leu-ILE-Gly-Gln (GTAGLIGQ; SEQ ID NO:1), (PA-C16-CA); wherein the second peptide amphiphile comprises comprises a hydrophilic peptide sequence and a hydrophobic tail, wherein the hydrophobic tail comprises a moiety having optionally substituted C16 alkyl chain, wherein the hydrophilic peptide sequence comprises a nitric oxide producing donor sequence comprising an amino acid sequence Lys-Lys-Lys-Lys-Lys (KKKKK) (K5; SEQ ID NO:3) and also comprises a degrading sequence (DS) comprising an amino acid sequence Gly-Thr-Ala-Gly-Leu-ILE-Gly-Gln (GTAGLIGQ; SEQ ID NO:1), (PA-C16-K5), wherein one or more of the Lys residues of the second peptide amphiphile comprises a pendant amine group, wherein one or more molecules of NO is bound to at least one pendant amine group; c) PA-05-K5-NO; a hydrophilic peptide sequence and a hydrophobic tail, wherein the hydrophobic tail comprises a moiety having a substituted C5 alkyl chain, wherein the hydrophilic peptide sequence comprises a nitric oxide producing donor sequence, comprising the amino acid sequence Lys-Lys-Lys-Lys-Lys (KKKKK; SEQ ID NO:3) and wherein one or more of the Lys residues comprise a pendant amine group, wherein one or more molecules of NO is bound to at least one of the pendant amine groups; PA-C16-K5-RGDS-NO; a hydrophilic peptide sequence and a hydrophobic tail, wherein the hydrophobic tail comprises a moiety having optionally substituted C16 alkyl chain, wherein the hydrophilic peptide sequence comprises a nitric oxide producing donor sequence comprising an amino acid sequence Lys-Lys-Lys-Lys-Lys (KKKKK) (1(5; SEQ ID NO:3) and comprises a degrading sequence (DS) comprising an amino acid sequence Gly-Thr-Ala-Gly-Leu-ILE-Gly-Gln (GTAGLIGQ; SEQ ID NO:1), and further comprising a cell adhesive ligand comprising an amino acid sequence Arg-Gly-Asp-Ser (RGDS; SEQ ID NO:4), wherein herein one or more of the lysine residues comprise pendant amine groups that react with NO to form a diazeniumdiolate-modified peptide amphiphile; or PA-C16-K5-YIGSR-NO; a hydrophilic peptide sequence and a hydrophobic tail, wherein the hydrophobic tail comprises a moiety having optionally substituted C16 alkyl chain, wherein the hydrophilic peptide sequence comprises a nitric oxide producing donor sequence comprising an amino acid sequence Lys-Lys-Lys-Lys-Lys (KKKKK) (K5; SEQ ID NO:3) and comprises a degrading sequence (DS) comprising an amino acid sequence Gly-Thr-Ala-Gly-Leu-ILE-Gly-Gln (GTAGLIGQ; SEQ ID NO:1), and further comprising a cell adhesive ligand comprising an amino acid sequence Tyr-Ile-Gly-Ser-Arg (YIGSR; SEQ ID NO:2), wherein herein one or more of the lysine residues comprise pendant amine groups that react with NO to form a diazeniumdiolate-modified peptide amphiphile.

In some aspects, the disclosed compositions comprise two or more antibiotics.

### D. Liposomes

Disclosed are liposomes comprising one or more of the peptide amphiphiles or compositions disclosed throughout.

For example, disclosed herein are liposomes comprising a peptide amphiphile comprising a hydrophilic peptide sequence and a hydrophobic tail, wherein the hydrophobic tail comprises a moiety having a substituted C5 alkyl chain, wherein the hydrophilic peptide sequence comprises a nitric oxide producing donor sequence.

Disclosed herein are liposomes comprising peptide amphiphiles comprising a hydrophilic peptide sequence and a hydrophobic tail, wherein the hydrophobic tail comprises a moiety having a substituted C5 alkyl chain, wherein the hydrophilic peptide sequence comprises a nitric oxide producing donor sequence wherein the nitric oxide producing donor sequence comprises the amino acid sequence Lys-Lys-Lys-Lys-Lys (KKKKK) (K5; SEQ ID NO:3).

Disclosed herein are liposomes comprising peptide amphiphile comprising a hydrophilic peptide sequence and a hydrophobic tail, wherein the hydrophobic tail comprises a moiety having a substituted C5 alkyl chain, wherein the hydrophilic peptide sequence comprises a nitric oxide producing donor sequence wherein the nitric oxide producing donor sequence comprises the amino acid sequence Lys-Lys-Lys-Lys-Lys (KKKKK) (K5; SEQ ID NO:3) and wherein one or more of the Lys residues comprise a pendant amine group.

Disclosed herein are liposomes comprising peptide amphiphiles comprising a hydrophilic peptide sequence and a hydrophobic tail, wherein the hydrophobic tail comprises a moiety having a substituted C16 alkyl chain, wherein the hydrophilic peptide sequence comprises a nitric oxide producing donor sequence comprising an amino acid sequence Lys-Lys-Lys-Lys-Lys (KKKKK; SEQ ID NO:3) and a degrading sequence (DS) comprising an amino acid sequence Gly-Thr-Ala-Gly-Leu-ILE-Gly-Gln (GTAGLIGQ; SEQ ID NO:1), wherein one or more of the lysine residues comprise a pendant amine group. In some aspects, the peptide amphiphiles comprise one or more molecules of nitric oxide bound to at least one pendant amine group.

Disclosed herein are liposomes comprising mixtures of peptide amphiphiles. In an aspect, disclosed herein are compositions comprising a mixture of two peptide amphiphiles, wherein the first peptide amphiphile comprises a hydrophilic peptide sequence and a hydrophobic tail, wherein the hydrophobic tail comprises a moiety having a substituted C16 alkyl chain, wherein the hydrophilic peptide sequence comprises an endothelial cell-adhesive sequence (CA) comprising an amino acid sequence Tyr-Ile-Gly-Ser-Arg (YIGSR; SEQ ID NO:2) and a degrading sequence (DS) comprising an amino acid sequence Gly-Thr-Ala-Gly-Leu-ILE-Gly-Gln (GTAGLIGQ; SEQ ID NO:1), and wherein the second peptide amphiphile comprises a hydrophilic peptide sequence and a hydrophobic tail, wherein the hydrophobic tail comprises a moiety having optionally substituted C16 alkyl chain, wherein the hydrophilic peptide sequence comprises a nitric oxide producing donor sequence comprising an amino acid sequence Lys-Lys-Lys-Lys-Lys (KKKKK) (1(.5; SEQ ID NO:3) and also comprises a degrading sequence (DS) comprising an amino acid sequence Gly-Thr-Ala-Gly-Leu-ILE-Gly-Gln (GTAGLIGQ; SEQ ID NO:1). In some aspects, the peptide amphiphiles comprise one or more molecules of nitric oxide bound to at least one pendant amine group.

Disclosed herein are liposomes comprising peptide amphiphiles comprising a hydrophilic peptide sequence and a hydrophobic tail, wherein the hydrophobic tail comprises a moiety having a substituted C5 alkyl chain, wherein the hydrophilic peptide sequence comprises a nitric oxide producing donor sequence, comprising the amino acid sequence Lys-Lys-Lys-Lys-Lys (KKKKK; SEQ ID NO:3) and wherein one or more of the Lys residues comprise a pendant amine group. In some aspects, the peptide amphiphiles comprise one or more molecules of nitric oxide bound to at least one pendant amine group.

Also disclosed are liposomes comprising a peptide amphiphile in combination with a pharmaceutically active agent (e.g., therapeutic or antibiotic). For example, disclosed are liposomes comprising a peptide amphiphile comprising a hydrophilic peptide sequence and a hydrophobic tail, wherein the hydrophobic tail comprises a moiety having a substituted C5 alkyl chain, wherein the hydrophilic peptide sequence comprises a nitric oxide producing donor sequence. Another example disclosed herein is liposomes comprising one or more peptide amphiphiles and a therapeutic drug, wherein the one or more peptide amphiphile comprise at least one peptide amphiphile comprising a) PA-C16-K5-NO; b) PA-C16-YK5-NO; or c) PA-05-K5-NO.

In some aspects, the liposome comprises cholesterol and a lipid. In some aspects, the lipid is 1,2-distearoyl-snglycero-3-phosphocholine (DSPC), 1,2-dipalmitoyl-snglycero-3-phosphocholine (DPPC), 1, 2-Distearoyl-sn-glycero-3-phosphoethanolamine-poly (ethylene glycol) (DSPE-PEG), Dioleoyl-3-trimethylammonium propane (DOTAP), dioleoylphosphatidylglycerol (DOPG), dioleoylphosphatidylcholine (DOPC), dimyristoylphosphatidylcholine (DMPC), dioleoylphosphatidylserine (DOPS), palmitoyloleyolphosphatidylglycerol (POPG), dioleoylphosphatidylethanolamine (DOPE), dipalmitoyl phosphatidyl ethanolamine (DPPE), dimyristoylphosphoethanolamine (DMPE), distearoyl-phosphatidy 1-ethanolamine (DSPE), palmitoyloleoyl-phosphatidylethanolamine (POPE), palmitoyloleoylphosphatidylcholine (POPC), egg phosphatidylcholine (EPC), distearoylphosphatidylcholine (DSPC), dipalmitoylphosphatidylcholine (DPPC), dipalmitoylphosphatidylglycerol (DPPG), DSPG, palmitoyloleyolphosphatidylglycerol (POPG), palmitoyloleoyl-phosphatidylethanolamine (POPE), l-stearoyl-2-oleoylphosphatidyethanolamine (SOPE), DSPE-(polyethylene glycol) PEG, DMPE-PEG, DPPE-PEG or DOPE-PEG.

In some aspects, the liposome has a diameter of about 40 nm to about 200 nm.

In some aspects, the pharmaceutically active agent is a therapeutic drug. In some aspects, the therapeutic drug is sirolimus, everolimus, paclitaxel, colchicine, a statin, or miRNA. Thus, disclosed are liposomes comprising one or more of the peptide amphiphiles and one or more of sirolimus, everolimus, paclitaxel, colchicine, statins, or miRNA. In some aspect, the liposome is a sirolimus releasing liposome, everolimus releasing liposome, paclitaxel releasing liposome, colchicine releasing liposome, statin releasing liposome, miRNA releasing liposome.

In some aspects, the pharmaceutically active agent is an antibiotic. In some aspects, the antibiotic is gentamicin or vancomycin. Thus, disclosed are liposomes comprising one or more of the peptide amphiphiles and one or more of gentamicin and vancomycin. In some aspect, the liposome is a gentamicin releasing liposome or vancomycin releasing liposome.

Disclosed are compositions comprising one or more of the liposomes described herein.

### E. Gels

Disclosed are compositions comprising only one peptide amphiphile. In some aspect, the peptide amphiphile is in the form of gel. In some aspects, disclosed are compositions comprising the peptide amphiphile (PA-C16-K5-NO) in the form of a gel, wherein the peptide amphiphile a hydrophilic peptide sequence and a hydrophobic tail, wherein the hydrophobic tail comprises a moiety having an optionally substituted C16 alkyl chain, wherein the hydrophilic peptide sequence comprises a nitric oxide producing donor sequence comprising an amino acid sequence KKKKK (SEQ ID NO:3) and also comprises a degrading sequence (DS) comprising an amino acid sequence GTAGLIGQ (SEQ ID NO:1), wherein one or more of the Lys residues of the peptide amphiphile comprises a pendant amine group, wherein one or more molecules of nitric oxide is bound to at least one pendant amine group. In some aspects, the presence of calcium chloride forms a gel. Therefore, in some aspects, the compositions can be a gel. Also disclosed are gels comprising a composition comprising PA-C16-1(5-NO and calcium chloride.

Disclosed are compositions comprising two or more peptide amphiphiles and calcium chloride, wherein at least one peptide amphiphile comprises one or more molecules of NO. In some aspects, the presence of the calcium chloride forms a gel. Therefore, in some aspects, the compositions can be a gel. Thus, also disclosed are gels comprising a composition comprising two or more peptide amphiphiles and calcium chloride, wherein at least one peptide amphiphile comprises one or more molecule of NO.

In some aspects, the two or more peptide amphiphiles are different peptide amphiphiles.

In some aspects, a first peptide can comprise peptide amphiphile comprising PA-C16-YK5-NO and a second peptide amphiphile can comprises peptide amphiphile comprising PA-C16-DS. Disclosed herein are compositions comprising two or more peptide amphiphiles and calcium chloride, wherein the first peptide amphiphile comprises a peptide amphiphile comprising a mixture of two peptide amphiphiles, wherein the first peptide amphiphile comprises a hydrophilic peptide sequence and a hydrophobic tail, wherein the hydrophobic tail comprises a moiety having a substituted C16 alkyl chain, wherein the hydrophilic peptide sequence comprises an endothelial cell-adhesive sequence (CA) comprising an amino acid sequence YIGSR (SEQ ID NO:2) and a degrading sequence (DS) comprising an amino acid sequence GTAGLIGQ (SEQ ID NO:1), (PA-C16-CA); wherein the second peptide amphiphile comprises a hydrophilic peptide sequence and a hydrophobic tail, wherein the hydrophobic tail comprises a moiety having optionally substituted C16 alkyl chain, wherein the hydrophilic peptide sequence comprises a nitric oxide producing donor sequence comprising an amino acid sequence KKKKK (SEQ ID NO:3) and also comprises a degrading sequence (DS) comprising an amino acid sequence GTAGLIGQ (SEQ ID NO:1), (PA-C16-1(5), wherein one or more of the Lys residues of the second peptide amphiphile comprises a pendant amine group, wherein one or more molecules of NO is bound to at least one pendant amine group; and the second peptide (PA-C16-DS) of the composition comprises a hydrophilic peptide sequence and a hydrophobic tail, wherein the hydrophobic tail comprises a moiety having a substituted C16 alkyl chain, wherein the hydrophilic peptide sequence comprises a degrading sequence (DS) comprising an amino acid sequence GTAGLIGQ (SEQ ID NO:1), wherein the degradation sequence comprises a matrix metalloprotease (MMP) specific cleavage site.

In some aspects, a first peptide can comprises peptide amphiphile comprising PA-C16-K5-NO and a peptide amphiphile comprising PA-C16-DS. Disclosed herein are compositions comprising two or more peptide amphiphiles and calcium chloride, wherein the first peptide amphiphile (PA-C16-K5-NO) comprises a peptide amphiphile comprising a hydrophilic peptide sequence and a hydrophobic tail, wherein the hydrophobic tail comprises a moiety having a substituted C16 alkyl chain, wherein the hydrophilic peptide sequence comprises a nitric oxide producing donor sequence comprising an amino acid sequence Lys-Lys-Lys-Lys-Lys (KKKKK; SEQ ID NO:3) and a degrading sequence (DS) comprising an amino acid sequence Gly-Thr-Ala-Gly-Leu-Ile-Gly-Gln (GTAGLIGQ; SEQ ID NO:1), wherein one or more of the lysine residues comprise a pendant amine group, wherein one or more molecules of NO is bound to at least one pendant amine group; and the second peptide (PA-C16-DS) of the composition comprises a hydrophilic peptide sequence and a hydrophobic tail, wherein the hydrophobic tail comprises a moiety having a substituted C16 alkyl chain, wherein the hydrophilic peptide sequence comprises a degrading sequence (DS) comprising an amino acid sequence GTAGLIGQ (SEQ ID NO:1), wherein the degradation sequence comprises a matrix metalloprotease (MMP) specific cleavage site.

In some aspects, a first peptide can comprise peptide amphiphile comprising PA-05-K5-NO and a second peptide amphiphile can comprise peptide amphiphile comprising PA-C16-DS. Disclosed herein are compositions comprising two or more peptide amphiphiles and calcium chloride, wherein the first peptide amphiphile (PA-05-K5-NO) comprises a peptide amphiphile comprising a hydrophilic peptide sequence and a hydrophobic tail, wherein the hydrophobic tail comprises a moiety having a substituted C5 alkyl chain, wherein the hydrophilic peptide sequence comprises a nitric oxide producing donor sequence, wherein the nitric oxide producing donor sequence comprises the amino acid sequence KKKKK (SEQ ID NO:3), wherein one or more of the Lys residues comprise a pendant amine group, wherein the pendant amine groups can react with Nitric Oxide to form a diazeniumdiolate-modified peptide amphiphile, wherein the diazeniumdiolate is a NO donor and comprises one or more molecules of NO. and a second peptide (PA-C16-DS) of the composition comprises a hydrophilic peptide sequence and a hydrophobic tail, wherein the hydrophobic tail comprises a moiety having substituted C16 alkyl chain, wherein the hydrophilic peptide sequence comprises a degrading sequence (DS) comprising an amino acid sequence GTAGLIGQ (SEQ ID NO:1), wherein the degradation sequence comprises a MMP specific cleavage site.

Disclosed are gels comprising two or more peptide amphiphiles and calcium chloride, wherein the first peptide is PA-C16-YK5-NO, and the second peptide is PA-C16-DS. Disclosed are gels comprising two or more peptide amphiphiles and calcium chloride, wherein a first peptide is PA-C16-1(5-NO and a second peptide is PA-C16-DS. Also, disclosed are gels comprising two or more peptide amphiphiles and calcium chloride, wherein a first peptide is PA-05-K5-NO, and a second peptide is PA-C16-DS.

In some aspects, the compositions further comprise a pharmaceutically active agent. In some aspects, the pharmaceutically active agent can be encapsulated in a liposome forming a therapeutic drug releasing liposome or antibiotic releasing liposome. In some aspects, the pharmaceutically active agent is a therapeutic drug or a therapeutic drug releasing liposome for cardiovascular disease. In some aspects, the therapeutic drug is one or more of sirolimus, everolimus, paclitaxel, colchicine, statins, and miRNA. In some aspects, the pharmaceutically active agent is an antibiotic or an antibiotic releasing liposome for treating infection. In some aspects, the antibiotic is gentamicin or vancomycin.

Thus, for example, disclosed are gels comprising two or more peptide amphiphiles, a pharmaceutically active agent, and calcium chloride, wherein the first peptide is PA-C16-YK5-NO, and the second peptide is PA-C16-DS, and wherein the pharmaceutically active agent is sirolimus, everolimus, colchicine, statins, or miRNA.

In some aspects, the gels described herein comprise two or more peptide amphiphiles and calcium chloride but no pharmaceutically active agent. In some aspects, the gels disclosed herein can be used to treat arteriovenous fistula failure.

In some aspects, the gels described herein comprise two or more peptide amphiphiles, calcium chloride, and a pharmaceutically active agent. In some aspects, these gels can be used to treat a disease that the pharmaceutically active agent is specific for. For example, cardiovascular disease can be treated if sirolimus, everolimus, paclitaxel, colchicine, statins, miRNA or a liposome releasing one or more of sirolimus, everolimus, paclitaxel, colchicine, statins, or miRNA are the pharmaceutically active agent.

Disclosed are gels comprising one or more of the peptides described herein, one or more of the compositions described herein, or one or more of the liposomes described herein.

### F. Medical Devices

Disclosed are medical devices coated with one or more of the peptide amphiphiles, compositions, liposomes, or gels described herein.

In some aspects, the medical device is a cardiovascular medical device. For example, a cardiovascular medical device can be a vascular stent, vascular graft, catheter, pacemaker, or heart valve.

In some aspects, the medical device can be, but is not limited to, coronary stents, drug eluting balloons, brain aneurysm coils, brain flow diverters, total knee arthroplasty, total disc arthroplasty, total hip arthroplasty, or percutaneous osseointegrated prostheses (POP).

In some aspects, a medical device can be any device known or identified for use inside a subject's body. Preferably, the medical device is one that is inserted into the cardiovascular system. In addition, the medical device can comprise any material suitable for use as a surgical implant.

Percutaneous coronary intervention has been used to treat atherosclerotic plaques clinically, which sometimes lead to barotrauma with injury to the endothelium and the vessel wall. Restenosis happens after standard balloon angioplasty, primarily due to elastic recoil of the vessel wall. Restenosis detected using coronary angiography is commonly defined as a binary event with re-narrowing of ≥50% of the vessel diameter. Although BMS demonstrated effectiveness in treating coronary artery disease, in-stent restenosis represented the central issue of BMS. Moreover, BMS implantation is associated with a high (nearly 40%) recurrence of binary restenosis. In addition, it has some critical long-term disadvantages, including limiting normal vasomotion and adaptive arterial remodeling, preclusion of bypass surgery, and persistent, chronic foreign body reaction that elicits inflammation from long-term metallic implants.

Thus, to address the issue of bare-metal stent (BMS), another type of stents, known as drug-eluting stents, have been developed. Drug-eluting stents (DES) can elute anti-proliferative agents, reducing in-stent restenosis. Current anti-proliferaive agents include everolimus, sirolimus, and biolimus. Current generation DES includes both durable and biodegradable polymer-coated stents, and the lifelong presence of durable polymer is reported to be related to chronic inflammation and neoatherosclerosis. Examples of drug-eluting stents include sirolimus-eluting stents such as Orsiro, COMBO Plus, and Ultimaster Tansei from BIOTRONIK, OrbusNeich, and Terumo. Despite DES demonstrating significant efficacy in suppressing restenosis; however, sirolimus released from DES is associated with a higher risk of endothelial apoptosis and incomplete re-endothelialization. Moreover, recent studies have revealed that the clinically used dose of sirolimus and its analogs cause serious adverse effects, including 1) damage to the endothelium, and 2) inflammation responses to the polymer coating that delivers sirolimus, and 3) the persistent risk of late thrombosis. Thus, it is imperative to develop effective approaches, such as the delivery of other agents along with sirolimus, to limit the adverse effects of sirolimus or its analogs at their currently used dose.

In addition to anti-proliferative agents, one option to avoid implanting multiple metallic layers of stents into ISR could be bioresorbable scaffolds (BRS), which, compared with DEB, can achieve more significant acute gain and prevent restenotic tissue prolapse and cover any edge dissection. So far, the BRS has been made primarily from PLLA, followed by magnesium and iron. Because PLLA has lower tensile and mechanical strength, stiffness, and ductility than steel or CoCr, scaffolds made from PLLA have thicker struts and a wider strut profile than metallic stents to improve tensile and radial strength. The most typical example of BRS is the Abbott Bioresorbable Vascular Scaffold (BVS) developed by Abbott Vascular, consisting of a poly d,llactide (PDLLA) backbone with a coating in a 1:1 ratio with everolimus. However, in 2017, based on a meta-analysis of 2-year outcomes of seven randomized clinical trials, Abbott Bioresorbable Vascular Scaffold was shown to have significantly higher rates of target-vessel myocardial infarction and stent thrombosis than DES, the everolimus-eluting, cobalt-chromium metallic stent. Thus, Abbott Bioresorbable Vascular Scaffold was withdrawn from the market. In addition to Abbott Bioresorbable Vascular Scaffold (BVS), other BVSs are under investigation, such as DESolve Nx from Elixir Medical, Fantom from REVA Medical, Magmaris from Biotronik, and XINSORB from HuaAn Biotechnology Coronary stents were developed to prevent the arterial recoiling process and acute closure due to arterial dissection which limited the effectiveness of coronary balloon angioplasty. Importantly, BVS can be coated with various drugs to functionalize as DES.

Disclosed herein are medical devices, such as a stents that can comprise titanium alloy. In an aspect, the medical device can comprise cobalt-cromium. The medical device can comprise nickel-titanium. In an aspect, the medical device can comprise a biodegradable polymer. In some aspects, the medical device is a vascular stent. In some aspects, the stent is a drug-eluting stent. For example, the stent can be a sirolimus-eluting stent or a paclitaxel-eluting stent.

The skilled person in the field can appreciate additional medical devices for use with the disclosed NO-releasing nanomaterials and a pharmaceutically active agent. Preferably, the medical device administered to a tissue or organ of the body usually comprises a natural endothelium. For example, in some aspects, the medical device is a vascular graft in some aspects. In some aspects, the medical device is a catheter. In some aspects, the medical device is a pacemaker. In some aspects, the medical device is a heart valve.

### G. Methods of Treating

Disclosed are methods of preventing infection in a subject comprising administering to the subject a therapeutically effective amount of one or more of the peptide amphiphiles, mixtures of the peptide amphiphiles, compositions, liposomes, gels, or medical devices described herein to a subject in need thereof.

Disclosed are methods of treating a subject in need thereof comprising administering one or more of the medical devices described herein to a subject during osseointegration.

Disclosed are methods of increasing vascularization in a subject comprising administering one or more of the gels or the medical devices described herein to a subject in need thereof.

Disclosed are methods of treating inflammation in a subject comprising administering one or more of the gels or the medical devices described herein to a subject in need thereof.

Disclosed are methods of treating atherosclerosis in a subject comprising administering to the subject a therapeutically effective amount of one or more of the compositions, liposomes, gels, or medical devices described herein to a subject in need thereof.

Disclosed are methods of treating cardiovascular disease in a subject comprising administering to the subject a therapeutically effective amount of one or more of the compositions, liposomes, gels, or medical devices described herein to a subject in need thereof. For example, disclosed are methods of treating cardiovascular disease in a subject comprising administering to the subject a therapeutically effective amount of a composition comprising a NO-releasing peptide amphiphile, drug or drug releasing liposomes including sirolimus and/or everolimus, paclitaxel, colchicine, and peptide amphiphile PA-C16-DS that can form a gel when CaCl₂ is applied.

Disclosed are methods of treating arteriovenous fistula failure in a subject, comprising administering to the subject a therapeutically effective amount of one or more of the peptide amphiphiles, compositions, liposomes, gels, or medical device described herein to a subject in need thereof. In some aspects, the gel comprises a first peptide and a second peptide, wherein the first peptide is the peptide PA-05-K5-NO, wherein the second peptide (PA-C16-DS) comprises a hydrophilic peptide sequence and a hydrophobic tail, wherein the hydrophobic tail comprises a moiety having a substituted C16 alkyl chain, wherein the hydrophilic peptide sequence comprises a degrading sequence (DS) comprising an amino acid sequence GTAGLIGQ (SEQ ID NO:1), wherein the degradation sequence comprises a matrix metalloprotease (MMP) specific cleavage site. In some aspects, treating arteriovenous fistula failure in a subject can be performed by administering to the subject a therapeutically effective amount of one or more of the peptide amphiphiles, compositions, liposomes, gels, or medical device, wherein the peptide amphiphile comprises PA-C16-YK5-NO and PA-C16-DS or PA-C16-K5-NO and PA-C16-DS.

### 1. Use of Disclosed Medical Devices

Disclosed are medical devices coated with the one or more of the disclosed compositions comprising NO-releasing nanomaterials and therapeutic drugs. Any of the disclosed medical devices can be used to treat subjects in need thereof.

Disclosed are methods of implanting (e.g. administering) the disclosed coated medical devices into a subject. Thus, in one aspect, the disclosed methods can comprise the steps of implanting a composition comprising a medical device (e.g., stent, vascular graft, catheter, pacemaker, or heart valve) coated with NO-releasing nanomaterials and a pharmaceutical agent into a subject. In some aspects, a medical device is coated with NO-releasing nanomaterials and a pharmaceutical agent prior to implanting into a subject. In a further aspect, a method can comprise the step of coating NO-releasing nanomaterials and a pharmaceutical agent onto a medical device after implantation into a subject.

### 2. Use of Disclosed Gels

As disclosed here, the composition disclosed herein can be in the form of a gel and the gel can be used to treat a subject in need thereof. In some aspects, the NO-releasing gel can be delivered to a subject through injection. In some aspects, the composition comprises a NO-releasing peptide amphiphile, wherein the NO-releasing peptide amphiphile can form a NO-releasing gel. In an aspect, disclosed is a NO-releasing gel comprising the NO-releasing peptide amphiphile (PA-C16-K5-NO). In some aspects, the composition comprises a NO-releasing peptide amphiphile and peptide amphiphile PA-C16-DS that can form a NO-releasing gel when CaCl₂ is applied. In one example, a NO-releasing gel comprising NO-releasing peptide amphiphile (PA-C16-YK5-NO) and peptide amphiphile PA-C16-DS and CaCl₂ was applied perivascularly at the arteriovenous anastomosis immediately following rat AVF creation. The NO-releasing nanomatrix gel inhibited intimal hyperplasia formation (more than 70% reduction), as well as improved vascular outward remodeling (increased vein diameter) and hemodynamic adaptation (lower wall shear stress approaching the preoperative level and less vorticity).

In addition, a method comprises the step of making the gelled macromolecules composed of NO-releasing peptide amphiphile, drugs, peptide amphiphile PA-C16-DS, and CaCl₂ before administration.

For peptide amphiphile PA-C16-DS, at neutral pH, the net negative charge can prevent the peptide amphiphile from self-assembling into gel structure; instead, the peptide amphiphiles remain amorphous. However, upon adding a high concentration of Ca⁺², the repulsive negative charges can be eliminated, and the peptide amphiphile undergoes physical cross-linking to provide the gelled macrostructure. The methods for administering NO-releasing gel into a subject include but are not limited to, oral administration, transdermal administration, administration by inhalation, nasal administration, topical administration, intravaginal administration, ophthalmic administration, intraaural administration, intracerebral administration, rectal administration, and parenteral administration, including injectables such as intravenous administration, intra-arterial administration, intramuscular administration. In addition to treating AVF maturation failure, the NO-releasing gel is also promising for treating cardiovascular disease.

### 3. Cardiovascular Disease Treatment

Cardiovascular disease (CVD) is the leading cause of death in the United States. The most frequent CVD cause is atherosclerosis, which is an inflammatory disease. Current therapeutic options for treating cardiovascular atherosclerosis mostly are cholesterol-lowering drugs, such as statins. In addition, stents are the most implanted devices used to treat cardiovascular diseases. However, bare-metal stent (BMS) use remains limited by high rates of in-stent restenosis resulting from neointimal proliferation in response to vessel injury during stent deployment. To address this, drug-eluting stents (DES) coated with anti-proliferative agents such as sirolimus or sirolimus analogs have been developed to reduce restenosis by targeting the biochemical pathways of neointimal hyperplasia. However, sirolimus released from DES is associated with a higher risk of endothelial apoptosis and incomplete re-endothelialization. Moreover, recent studies have revealed that the clinically used dose of sirolimus and its analogs cause serious adverse effects, including 1) damage to the endothelium, 2) inflammation responses to the polymer coating that delivers sirolimus, and 3) the persistent risk of late thrombosis. Thus, it is imperative to develop effective approaches to limit the adverse effects of sirolimus or its analogs at their currently used dose.

Inflammation resolution has been studied as a potential novel approach for treating atherosclerosis. Colchicine, is an alkaloid extracted from Colchicum autumnale, Gloriosa superba, and other plants, with a chemical name (−)-N-[(7S, 12a5)-1,2,3,10-tetramethoxy-9-oxo-5,6,7,9-tetrahydrobenzo[a]heptalen-7-yl]-acetamide. It is a microtubule-disrupting agent clinically used to treat acute flares of gouty arthritis, familial Mediterranean fever (FMF), and Behçet's disease. In recent years, colchicine has been investigated as a potential therapeutic for treating CVDs, due to its potent anti-inflammatory properties. A recent meta-analysis of four major randomized controlled trials demonstrated that the use of colchicine was associated with a significant reduction in the primary composite endpoint of cardiovascular mortality, myocardial infarction, ischemic stroke, and urgent revascularization. Moreover, evidence from recent clinic trial studies demonstrated that low-dose colchicine could decrease the risk of acute cardiovascular events in patients with chronic coronary disease. Colchicine is therapeutic to improve outcomes in coronary atherosclerosis, pericarditis, atrial fibrillation and heart failure.

Another potential class of drugs for local delivery to reduce inflammation are statins. Statins provide lipid-lowering effects and reduce cardiovascular risk when administered systemically. However, they are often associated with systemic side effects, including muscle soreness, myopathy, liver damage and increased risk of diabetes development. Local delivery of statins might provide greater stain concentration in target tissues while minimizing side effects. Beneficial effects could include improved vascular endothelial function, cardiac regeneration, and improved re-endothelialization

In sum, the desire to reduce the delivered dose of sirolimus used as well as the potential benefits of NO, colchicine, and statins for CVD therapy, resulted in the disclosed co-delivery of NO with sirolimus, colchicine or statin for improving the treatment of CVD. This can improve efficacy and decrease potential adverse effects from delivering sirolimus or other medications.

As a solution to the problems associated with treating cardiovascular disease, disclosed are methods of treating a subject comprising administering to the subject a therapeutically effective amount of one or more of the peptide amphiphiles, mixtures of the peptide amphiphiles, compositions, liposomes, gels, or medical devices described herein to a subject in need thereof.

### 4. Amputation Treatment

Approximately 1.6 million people live with an amputation within the US, and amputation cases are expected to rise to approximately 3.6 million by 2050. Amputation is recommended for many reasons in the overall population, including peripheral artery disease (leading cause), severe trauma, tumor, or infection. Major lower limb amputations are much more frequent, with an estimated prevalence of nearly 40% of all limb loss. Transtibial is the most prevalent lower limb amputation at 52%, followed by transfemoral at 25%. The outcome of limb loss often leads to a decline in physical, social, and financial wellbeing. Returning to normal life and/or continuing pre-injury activities is challenged by conventional prosthetic techniques. However, current socket technology is associated with discomfort and functional limitations, especially in patients with short residual limbs or multiple limb loss. The conventional prosthetic sockets technology is unable to adapt to the dynamic residual limb as it atrophies over time and swells with heat or weight gain. In addition, socket prosthetics are prone to skin irritation, pain, and problems with prosthetic fixation. The issues of comfort and fit associated with the socket reduce the quality of life and mobility of the patient.

Percutaneous osseointegrated prostheses (POP) are a promising development for the limb-prosthesis interface involving the direct skeletal attachment of the prosthetic device. There are many variations of the implant system, the anchoring portion of the POP, including osseointegrated prostheses for the rehabilitation of amputees (OPRA), integral leg prosthesis (ILP), osseointegrated prosthetic limb (OPL), intraosseous transcutaneous amputation prosthesis (ITAP), keep walking advanced, and POP. To date, OPRA, ILP, and OPL are the only commercially available implant systems. POP improves donning and doffing, comfort, fit, skin irritation, range of motion, and osseo-proprioception over traditional socket prosthetics. Alongside the promising benefits of POP, significant risks are present at the bone-implant interface, including superficial and deep infection, inflammation, insufficient osseointegration, lack of vascularization, and implant loosening: 1) Infection: In screw implants, infection in soft tissue (Grade 1-2) occurs in 28% of cases and in bone in 5-13% (Grade 3). The introduction of bacteria to the implant site leads to the formation of an antibiotic-resistant biofilm and/or invasion into osteoblasts reducing bone tissue formation. 2) Inflammation: A study retrospectively assessing femoral implant patients found a 10-year cumulative risk for implant-associated osteomyelitis of 20% and a subsequent extraction in 9% of cases. Infection and biomaterial rejection are significant causes of inflammation at the implant and/or soft tissue site. It is critical for POP devices to avoid implementing immunogenic biomaterials to avoid rejection. To date, attempts to confront inflammation proactively have been confined to the aseptic operative technique. 3) Vascularization: Vascularization at the implant site fosters an environment rich in nutrients, biomolecules, and cellularity to encourage bone development, regeneration, and remodeling. 4) Osseointegration: Reports indicate the incidence of screw implant loosening within the bone varied from 3-23% of reported cases. Titanium has been implemented in the majority of modern POP designs due to its strength and biocompatibility. However, long-term studies have shown that a layer of fibrous tissue forms between the implant and bone. To further improve implant surface characteristics, hydroxyapatite is often coated onto the implant surface to torm a stronger bond and quicker fixation with bone. However, plasma spraying hydroxyapatite onto the implant surface requires a high temperature leading to an elevated bio-dissolution rate.

Although there have been efforts to improve antibacterial effects or osseointegration of POP, there are no studies to improve multiple aspects, including angiogenesis and inflammation. The disclosed peptide Amphiphiles and uses thereof overcome current issues in POP by ameliorating infection, suppressing inflammation, and enhancing vascularization and osseointegration.

The direct incorporation of NO into POP intended for osseointegration or bone healing has never been attempted. However, NO plays a role in promoting angiogenesis via recruiting perivascular and endothelial cells. NO has also been shown to affect vascular endothelial growth factor release during angiogenesis occurring in bone remodeling. In addition, NO promotes the synthesis of cGMP, thereby activating downstream pathways, including cGMP-dependent kinase II (cGKII), which is known to play an essential role in endochondral bone formation. Since healing of a bone fracture undergoes endochondral ossification, using the NO-releasing multifunctional nanomatrix can promote the osseointegration of POP.

For antibacterial effects, NO is known to act as a signaling molecule in promoting the detachment of biofilms but does not affect host cell viability. NO can protect host cells from oxidative damage by terminating lipid peroxidation reactions. Recently, concentration-dependent antibacterial effects of the NO-releasing Nanomatrix were demonstrated. For anti-inflammatory effects of NO, NO synthase systems are associated with macrophages' wound healing processes. The NO-releasing multifunctional nanomatrix significantly decreased monocyte adhesion and pro-inflammatory gene expression.

Therefore, to tackle the critical issues of infection, osseointegration, vascularization, and inflammation of current POP, the disclosed compositions comprising a multifunctional NO and low dose of antibiotics have been used. These compositions can be used for improved osseointegration of prosthetics and other orthopedic and dental implants to help promote healing and prevent infection.

As a solution to the problems associated with amputation, disclosed are methods of treating a subject comprising administering to the subject a therapeutically effective amount of one or more of the peptide amphiphiles, mixtures of the peptide amphiphiles, compositions, liposomes, gels, or medical devices described herein to a subject in need thereof.

### 5. Arteriovenous Fistulas Treatment

A working vascular access is the “lifeline” for hemodialysis patients. Over 700,000 patients with End-Stage Renal Disease (ESRD), and 70% of these patients utilize hemodialysis as their kidney replacement modality of choicel. A functioning and durable vascular access provides the conduit to achieve consistent dialysis therapy for the hemodialysis patient. Arteriovenous fistulas (AVFs), created by a direct anastomosis between a native artery and vein are the preferred type of vascular access because AVFs have substantially lower thrombosis rates, infection and healthcare-related expenditures if they mature successfully for dialysis. However, AVF maturation failure remains a significant cause of morbidity, mortality, and hospitalization among hemodialysis patients. Results from a multicenter randomized clinical trial in the United States reported that 60% of AVFs created in hemodialysis patients fail to mature for dialysis use. The annual costs of treating vascular access dysfunction are over two billion U.S. dollars, mainly because the high proportion of non-maturing AVFs result in frequent interventions to promote maturation and long-term dialysis catheter use. Despite the magnitude of AVF maturation failure, we presently have no effective therapies to prevent or treat this clinical problem. Thus, identifying therapeutic targets and developing local novel therapies to reduce AVF maturation failure in hemodialysis patients represents an unmet clinical need.

The endothelium plays an essential role in regulating AVF development. In addition to the benefits of NO for vascular remodeling discussed previously, endothelial-derived NO is a key vasodilator and signaling molecule in vascular remodeling, and it has also been shown to inhibit intimal hyperplasia in arterial injury models. Moreover, the overexpression of NOS3 reduced initial hyperplasia development in AVFs supported the hypothesis that increasing and sustaining local NO bioavailability—concurrent with AVF creation—can inhibit AVF maturation failure. Thus described herein are methods of administering NO, via peptide Amphiphiles, to the AVF. Importantly, it was demonstrated that the NO-releasing gel was successfully applied at the venous anastomosis of the rat AVF and reduced both venous intimal hyperplasia and a pro-inflammatory mediator and other markers of inflammation compared to the control nanomatrix gel (without NO) treated group by histological, biological analysis, and transcriptomics analysis. Thus, described herein is a local delivery of NO to address AVF maturation failure.

As a solution to the problems associated with treating AVF, disclosed are methods of treating a subject comprising administering to the subject a therapeutically effective amount of one or more of the peptide amphiphiles, mixtures of the peptide amphiphiles, compositions, liposomes, gels, or medical devices described herein to a subject in need thereof.

### H. Methods of Making Peptide Amphiphiles

The compositions disclosed herein and the compositions necessary to perform the disclosed methods can be made using any method known to those of skill in the art for that particular reagent or compound unless otherwise explicitly noted.

In an aspect, disclosed is a method of producing the disclosed peptide amphiphiles, such as SEQ ID NO:1 to SEQ ID NO:3, by linking two or more peptides or polypeptides together by protein chemistry techniques and solid peptide synthesis (SPPS). Particularly, peptides or polypeptides can be chemically synthesized using currently available laboratory equipment, either Fmoc (9-fluorenylmethyloxycarbonyl) or Boc (tert-butyloxycarbonoyl) chemistry. (Applied Biosystems, Inc., Foster City, Calif). One skilled in the art can readily appreciate that a peptide or polypeptide corresponding to the disclosed proteins, for example, can be synthesized by standard chemical reactions. The solid-phase peptide synthesis (SPPS) is traditionally carried out in the C→N direction. Most peptides are synthesized as C-terminal acids or amides. By peptide condensation reactions, these two fragments can be covalently joined via a peptide bond at their carboxyl and amino termini, respectively, to form an antibody or fragment thereof. Successful SPPS depends on the solid support, linker (between the solid support and the synthesized peptide), appropriately protected amino acids, coupling methodology, and protocol for cleaving the peptide from the solid support. The solid support often refers to commensally available resin. The term coupling refers to forming a peptide bond between two adjacent amino acids. The typical examples of in situ coupling reagents are N,N′-dicyclohexylcarbodiimide (DCC), and the related N,N′-diisopropylcarbodiimide (Rich and Singh, 1979). The generality of carbodiimide-mediated couplings is extended significantly. The use of either 1-hydroxybenzotriazole (HOBt) or 1-hydroxy-7-azabenzotriazole (HOAt) as an additive, either of which accelerates carbodiimide-mediated couplings, suppresses racemization, and inhibits dehydration of the carboxamide side chains of Asn and Gln to the corresponding nitriles. In addition to carborbodiimides, protocols involves chemicals, such as, hexafluorophosphate (PyAOP), O-benzotriazol-1-yl-N,N,N′,N′-tetramethyluronium hexafluorophosphate (HBTU), O-(7-azabenzotriazol-1-yl)-N,N,N′N′-tetramethyluronium hexafluorophosphate (HATU), O-(6-Chlorobenzotriazol-1-yl)-N, N,N′,N′- tetramethyluronium hexafluorophosphate (HCTU), leads to more rapid coupling kinetics than these obtained with carbodiimides

For resin cleavage, in some cases, a peptide or polypeptide can be synthesized and not cleaved from its synthesis resin, whereas the other fragment of a peptide or protein can be synthesized and subsequently cleaved from the resin, thereby exposing a terminal group that is functionally blocked on the other fragment.

It is contemplated that a disclosed peptide amphiphiles can be prepared by attachment of a hydrophobic moiety via conventional synthetic techniques. For example, a hydrophobic moiety can be attached to the N-terminus of the hydrophilic peptide. That is, hydrophobic electrophilic compounds (e.g., alkyl halide, carboxylic compound) can be reacted with the amine function

In further examples, a hydrophobic moiety can be attached at the C-terminus of the hydrophilic peptide. Hydrophobic nucleophilic compounds (e.g., alcohol, amine, thiol) can be reacted with the carboxylic function present at the C-terminus to provide a covalent linkage (e.g., ester, amide, thioesters). It is further contemplated that the carboxylic function present at the C-terminus can be derivatized or reduced prior to reaction. For example, the carboxylic function can be reduced to form an alcohol and subsequently reacted with one or more hydrophobic electrophilic compounds (e.g., alkyl halide, carboxylic compound) to provide a covalent linkage (e.g., ether, ester).

As readily understood by those of skill in the art, peptide sequences can comprise peptide residues having one or more pendant groups. The pendant groups, in various aspects, can comprise one or more nucleophilic moieties (e.g., amine, hydroxyl, thiol) or one or more electrophilic moieties (e.g., carboxylic function). Such moieties can be reacted in a manner analogous to that disclosed above for N-terminus and C-terminus of a disclosed hydrophilic peptide.

Disclosed are methods of making the peptide amphiphile PA-05-K5-NO comprising obtaining a peptide amphiphile comprising a nitric oxide (NO) producing donor sequence, wherein the peptide amphiphile comprises an N-terminus, alkylating the N-terminus of the peptide amphiphile with a hydrophobic moiety, wherein the alkylation comprises amination with a valeric acid, and conjugating NO to the peptide amphiphile. In some aspects, the peptide amphiphile that is obtained is the peptide amphiphile PA-05-K5 wherein the pendant amine groups can react with nitric oxide to form a diazeniumdiolate-modified peptide amphiphile.

Disclosed are methods of making a liposome comprising producing a lipid thin film, wherein the lipid thin film comprises cholesterol and lipids, wherein the lipids are 1,2-distearoyl-snglycero-3-phosphocholine (DSPC), 1,2-dipalmitoyl-snglycero-3-phosphocholine (DPPC), and 1, 2-Distearoyl-sn-glycero-3-phosphoethanolamine-poly (ethylene glycol) (DSPE-PEG), or Dioleoyl-3-trimethylammonium propane (DOTAP); hydrating the lipid thin film with a buffer solution, wherein the buffer solution comprises a peptide amphiphile; and extruding the buffer solution using a polycarbonate membrane filters. In some aspects, the peptide amphiphile is the peptide amphiphile of any of those described herein, particularly PA-05-K5-NO or PA-05-K5. In some aspects, extruding the buffer solution using a polycarbonate membrane filters comprises using 800 nm, 400 nm, and 200 nm polycarbonate membrane filters.

### I. Kits

The materials described above as well as other materials can be packaged together in any suitable combination as a kit useful for performing, or aiding in the performance of, the disclosed method. It is useful if the kit components in a given kit are designed and adapted for use together in the disclosed method. For example disclosed are kits comprising one or more of the peptide amphiphiles, compositions, liposomes, gels, medical devices, or combinations thereof described herein.

The disclosed kits can also include directions for making the medical devices coated in the disclosed peptide amphiphiles, compositions, gels or liposomes.

### EXAMPLES

The following examples are put forth so as to provide those of ordinary skill in the art with a complete disclosure and description of how the compounds, compositions, articles, devices and/or methods claimed herein are made and evaluated and are intended to be purely exemplary and are not intended to limit the disclosure. Efforts have been made to ensure accuracy concerning numbers (e.g., amounts, temperature, etc.), but some errors and deviations should be accounted for. Unless indicated otherwise, parts are parts by weight, the temperature is in ° C. or is at ambient temperature, and pressure is at or near atmospheric.

### A. Example 1

### 1. Synthesis of Peptide Amphiphile 2 (PA-C16-CA), Peptide Amphiphile 3 (PA-C16-K5), and Peptide Amphiphile 7 (PA-05-K5).

Peptide Amphiphiles 2 (PA-C16-CA), 3 (PA-C16-1(5), and 7 (PA-05-K5) have been synthesized based on our established protocols in our previous studies. Specifically, peptide amphiphile 2 or 3 consisting of MMP-2 sensitive sequences (GTAGLIGQ; SEQ ID NO: 1) with cell-adhesive sequence YIGSR (SEQ ID NO:2) (“PA-C16-CA”) or NO donor sequence KKKKK (SEQ ID NO:3) (“PA-C16-K5”) were synthesized using standard Fmoc-chemistry on an Advanced Chemtech Apex 396 peptide synthesizer, respectively. Alkylation was obtained by reacting N-termini of the peptides with 2 equivalents of palmitic acid, 2 equivalents of o-benzotriazole-N,N,N′,N′-tetramethyluroniumhexafluorophosphate (HBTU), and 4 equivalents of diisopropylethylamine (DiEA) in dimethylformamide (DMF) for 6 h at room temperature. Similarly, peptide amphiphile 7 (PA-05-K5), comprising NO donor sequence KKKKK (SEQ ID NO:3), was synthesized using the abovementioned approaches. However, in the alklyaltion, valeric acid can be used instead of palmitic acid.

After repeating the alkylation reaction, cleavage and deprotection of PA 2,3 and 7 were performed using a mixture of trifluoroacetic acid (TFA), deionized (DI) water, triisopropylsilane, and anisole in the ratio of 90:1:1:1 for 3 hours at room temperature. The solution was concentrated using a rotary evaporator. PAs were precipitated in cold ether, collected, and dried under a vacuum. The crude PAs were dissolved in DI water at a concentration of 2 wt %. The PAs were analyzed by matrix-assisted laser desorption ionization time-of-flight (MALDI-TOF) mass spectrometry. Based on the MALDI-TOF result, the molecular weight of PA-05-K5 is about 743 g/mol.

2. Self-Assembly of Peptide Amphiphiles 2 (PA-C16-CA) and Peptide Amphiphile 3 (PA-C16-K5) into Peptide Amphiphile 5 (PA-C16-YK5).

The self-assembly of peptide amphiphile 5 was reported in our previous studies. 1 wt % stock solutions for PA-C16-CA and PA-C16-K5 were prepared in DI water (pH 7.4) and mixed in a molar ratio of 9: 1 to form PA-C16-YK5.

3. Preparation of Peptide Amphiphile 6 (PA-C16-YK5-NO) and peptide Amphiphile 8 (PA-05-K5-NO)

Peptide amphiphile 6 (PA-C16-YK5-NO) and peptide amphiphile 8 (PA-05-K5-NO) were synthesized using peptide amphiphile 5 (PA-C16-YK5) or PA-05-K5-NO with scrubbed NO under argon gas. Scrubbing is a process by which a gas is passed through a large liquid surface area to remove unwanted impurities from the gas stream. Herein, the commercially available NO is passed through an alkaline solution which dissolves the unwanted higher nitrogen oxide species. Then, 1 wt % PA-C16-YK5 and PA-05-K5 aqueous solutions were reacted with scrubbed NO solution at room temperature under argon gas in 100 mL round bottom flask overnight to obtain PA-C16-YK5-NO or PA-05-K5-NO.

### 4. Size Analysis of Peptide Amphiphile 8 (PA-05-K5-NO) Using DLS

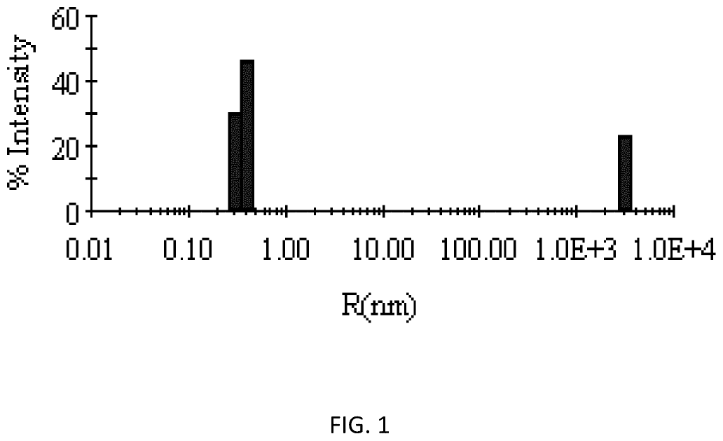

The size and size uniformity of PA-05-K5-NO was assessed using dynamic light scattering (DLS). PA-05-K5-NO were diluted in DI water, and lml was added to the cuvette (Eppendorf, Hamburg, Germany). The cuvette was loaded into the DLS (DynaPro), and the sample was read at 10% laser power using the Dynamics V6.3.40 software. The radius and polydispersity index (PDI) were recorded to evaluate size and distribution. As demonstrated in FIG. 1, the average radius of PA-05-K5-NO is 0.53±0.07 nm.

### B. Example 2 Liposome Fabrication

### 1. NO-Releasing Liposomes

i. Synthesis of NO-Releasing Liposomes (Peptide Amphiphile 8 (PA-05-K5-NO) Encapsulating Liposomes)

Briefly, DPPC, DOTAP, DSPE-PEG, and CHOL were dispersed in chloroform in a molar ratio of 55:25:3:27 in a 3 ml vial. The mixture was then left to dry overnight to create a thin lipid film using a vacuum. Next, the lipid thin film was hydrated with 0.025M HEPES solution with 1 wt % PA-05-K5-NO for 3 hours to form PA-05-K5-NO encapsulating liposomes. Liposomes were then extruded using 800 nm, 400 nm, and 200 nm polycarbonate membrane filters, respectively, to control size homogeneity. Finally, the unencapsulated PA-05-K5-NO was separated using the G-25 Sephadex column with the spin method.

ii. Transmission Electron Microscope (TEM) Imaging of PA-05-K5-NO Encapsulating Liposomes

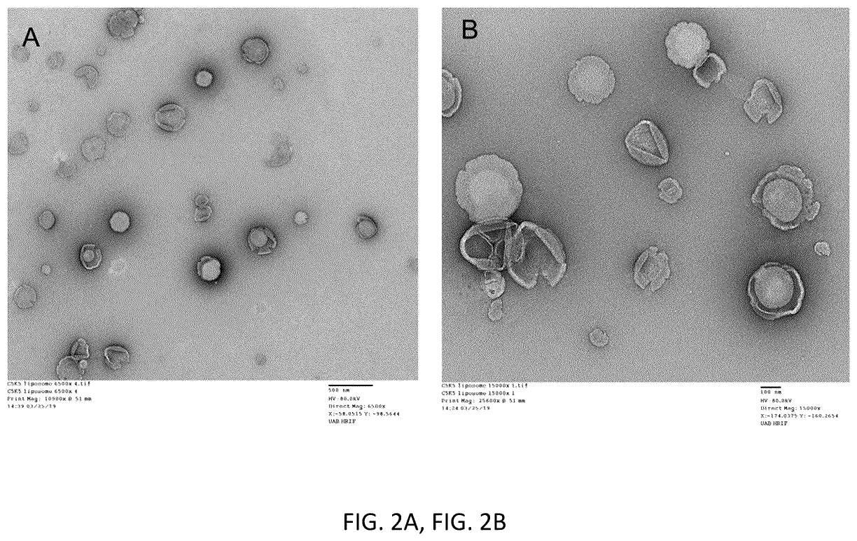

For TEM samples, 5 μl of liposome encapsulated with PA-05-K5-NO aqueous solution were cast on a carbon-coated formvar copper grid (400 mesh). This grid was dried overnight. Before imaging, the dried samples were negatively stained with 10 μl of 20% phosphotungstenic acid (PTA) for 30s. The samples were imaged on a FEI Tecnai T12 TEM microscope at 60 kV accelerating voltage. As demonstrated in FIG. 2A-2B, the liposomes encapsulated with PA-05-K5-NO demonstrated a spherical shape

iii. Size Analysis of PA-05-K5-NO Encapsulating Liposomes Using DLS

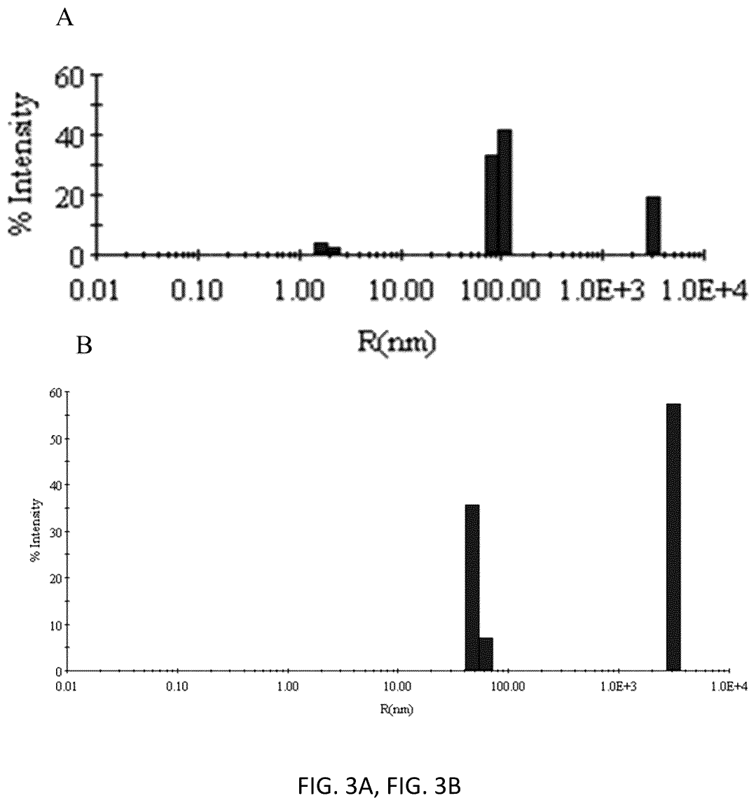

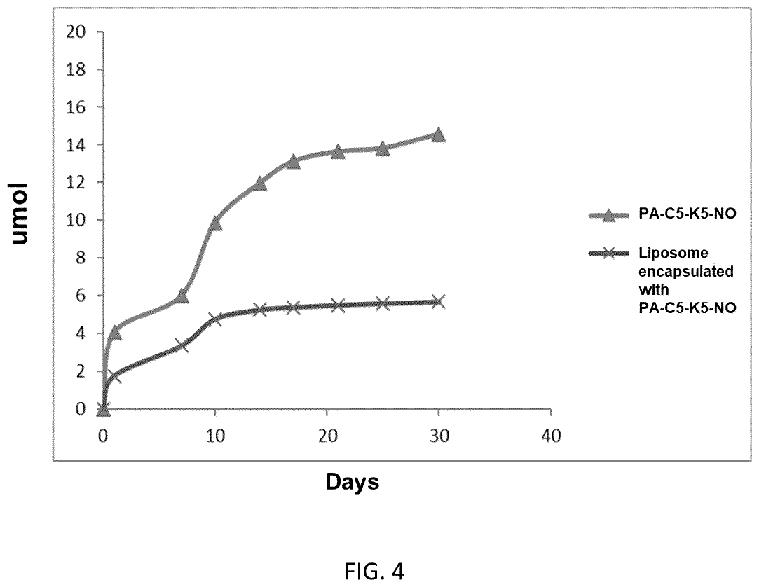

Size and size uniformity of NO-releasing liposomes was assessed using DLS). NO-releasing liposomes were diluted in DI water, and 1mL was added to the cuvette (Eppendorf, Hamburg, Germany). The cuvette was loaded into the DLS (DynaPro), and the sample was read at 10% laser power using the Dynamics V6.3.40 software. The radius and polydispersity index (PDI) were recorded to evaluate size and distribution. As demonstrated in FIG. 3, the average radius of NO-releasing liposomes is around 107.87±4.68 nm. Moreover, the NO-releasing liposomes are relatively stable, with a size of 105.47±12.66 nm after 7 days in the buffer (FIG. 4).

iv. Release of NO From PA-05-K5-NO Encapsulating Liposomes

The NO release kinetic from PA-05-K5-NO encapsulating liposomes was investigated.

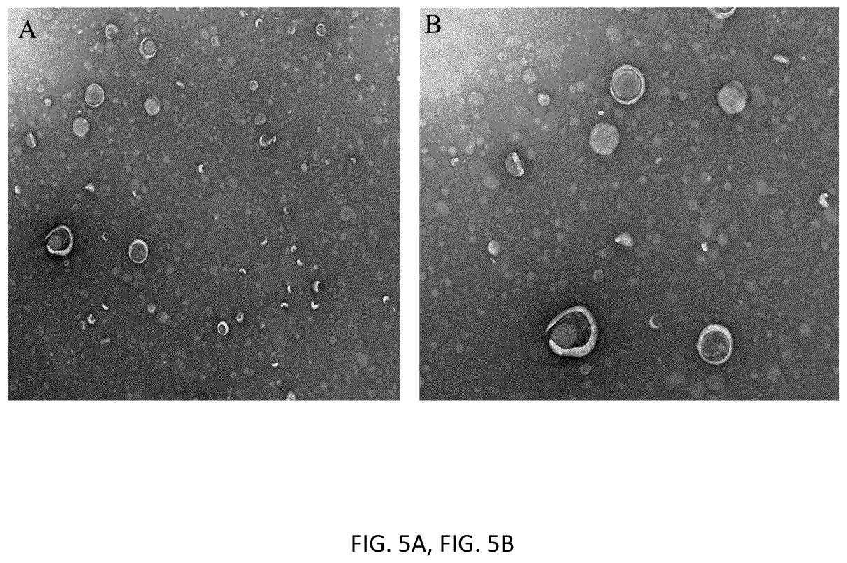

Result: as shown in FIG. 5, the rapid NO release was observed within the first 10 hours, the total release within the first 10 hours around 4 nmol.

v. Synthesis of PA-C16-K5-NO Encapsulating Liposomes

Briefly, DPPC, DOTAP, DSPE-PEG, and CHOL were dispersed in chloroform in a molar ratio of 55:25:3:27 in a 3 ml vial. The mixture was then left to dry overnight to create a thin lipid film using a vacuum. Next, the lipid thin film was hydrated with 0.025M HEPES solution with 1 wt % PA-C16-K5-NO for 3 hours to form PA-C16-K5-NO encapsulating liposomes. Liposomes were then extruded using 800 nm, 400 nm, and 200 nm polycarbonate membrane filters, respectively, to control size homogeneity. Finally, the unencapsulated PA-C16-K5-NO was separated using the G-25 Sephadex column with the spin method.

vi. Transmission Electron Microscope (TEM) Imaging of PA-C16-K5-NO Encapsulating Liposomes

For TEM samples, 5 μl of liposome encapsulated with PA-C16-K5-NO aqueous solution were cast on a carbon-coated formvar copper grid (400 mesh). This grid was dried overnight. Before imaging, the dried samples were negatively stained with 10 μl of 20% phosphotungstenic acid (PTA) for 30 s. The samples were imaged on a FEI Tecnai T12 TEM microscope at 60 kV accelerating voltage. As demonstrated in FIGS. 5A-5B, the liposomes encapsulated with PA-05-K5-NO demonstrated a spherical shape

vii. Size Analysis of PA-C16-K5-NO Encapsulating Liposomes Using DLS

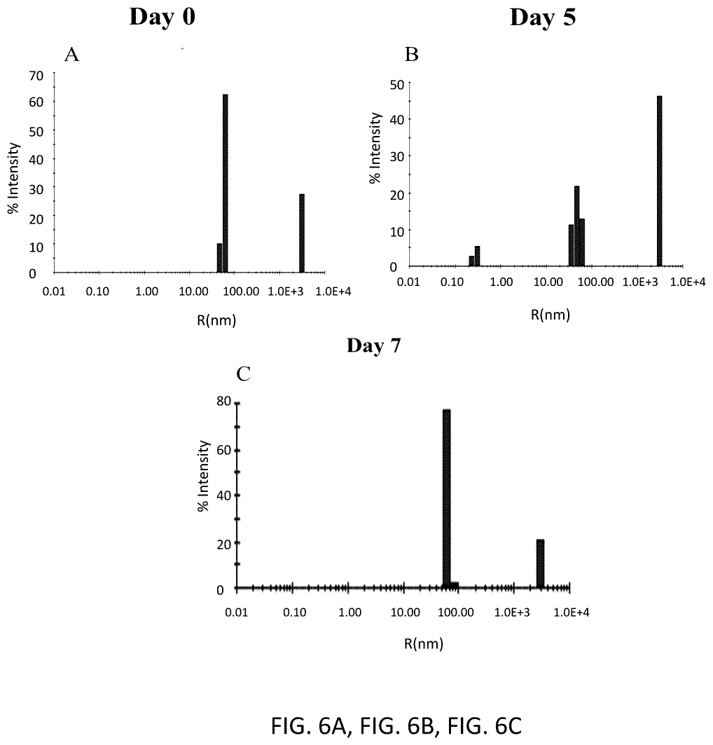

Size and size uniformity of PA-C16-K5-NO encapsulating liposomes was assessed using DLS). NO-releasing liposomes were diluted in DI water, and 1 mL was added to the cuvette (Eppendorf, Hamburg, Germany). The cuvette was loaded into the DLS (DynaPro), and the sample was read at 10% laser power using the Dynamics V6.3.40 software. The radius and polydispersity index (PDI) were recorded to evaluate size and distribution. As demonstrated in FIG. 6, the average radius of PA-C16-K5-NO encapsulating liposomes is around 81.9±6.43 nm. Moreover, the NO-releasing liposomes are relatively stable, with a size of 81.66±18 nm and 80.67±3.56 nm after 5 days and 7 days in the buffer, respectively.

2. microRNA146a-Releasing Liposomes
i. Synthesis of microRNA146a-Releasing Liposomes

DPPC, DOTAP, DSPE-PEG, and CHOL (molar ratio: 55:15:3:27; Avanti Polar Lipids) were dissolved in chloroform (1 ml) and dehydrated under vacuum overnight. The dehydrated lipids were then rehydrated with RNase free water containing microRNA146a (150 nM) for encapsulation for 2 hours. The liposome solution was extruded through 0.8 μm, 0.4 μm, and 0.2 μm pore polycarbonate membrane filters sequentially using an extruder set

ii. Characterization of microRNA146a-Releasing Liposomes

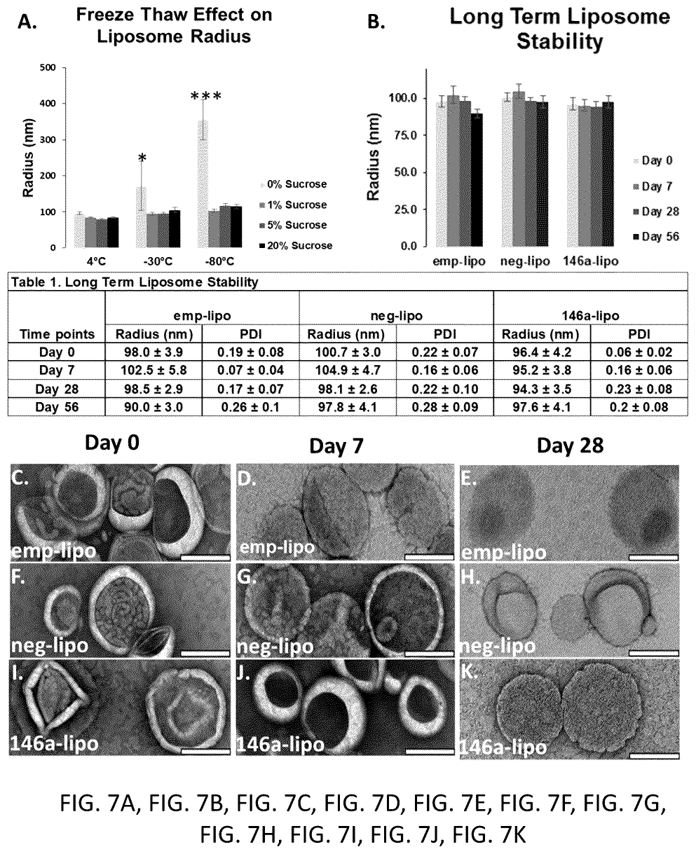

The storage condition, size and stability of microRNA146a-releasing liposomes were characterized and shown in FIG. 7. As shown in FIG. 7A, microRNA146a-releasing liposomes are better to storage at 4° C. with sucrose. TEM of the liposomes reflected these DLS results well with the 4° C. liposome having a uniform round shape. Long term stability of microRNA146a-releasing liposomes, at room temperature was also evaluated by DLS at 0, 7, 21, and 56 days and TEM imaging at 0, 7, and 28 days. At all time points, up to 28 days, the hydrodynamic sizes of the liposomes were approximately 90-100 nm, which suggested microRNA146a-releasing liposomes were stable and maintained their expected size of a 100 nm radius (FIG. 7B and Table 1). Additionally, TEM imaging did not show any aggregation or breakdown of the liposomes after 4 weeks (FIGS. 7C-K). These results indicated the liposomes may maintain stability and shelf-life for at least one month.

iii. Transfection of microRNA146a-Releasing Liposomes

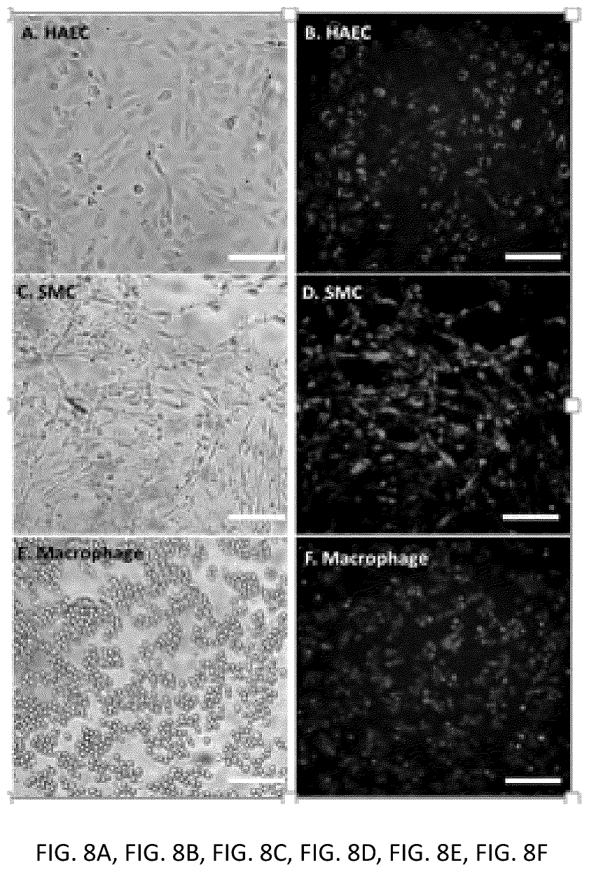

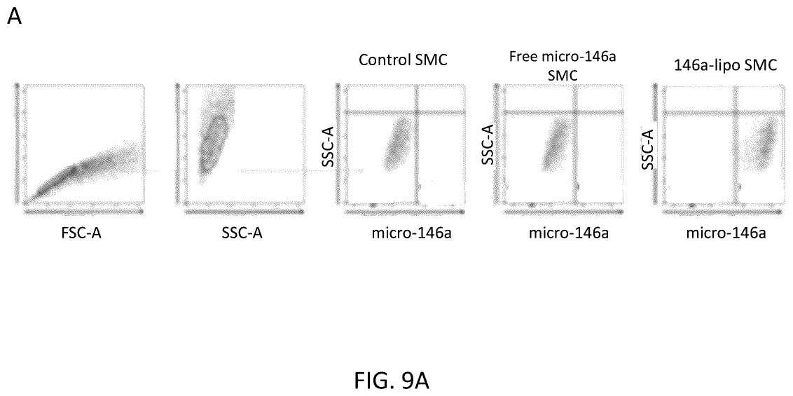

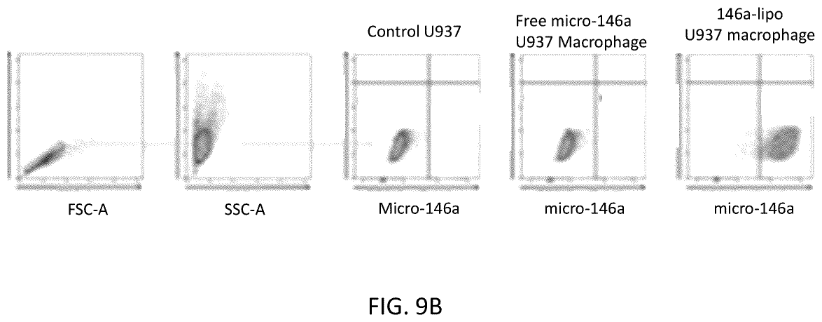

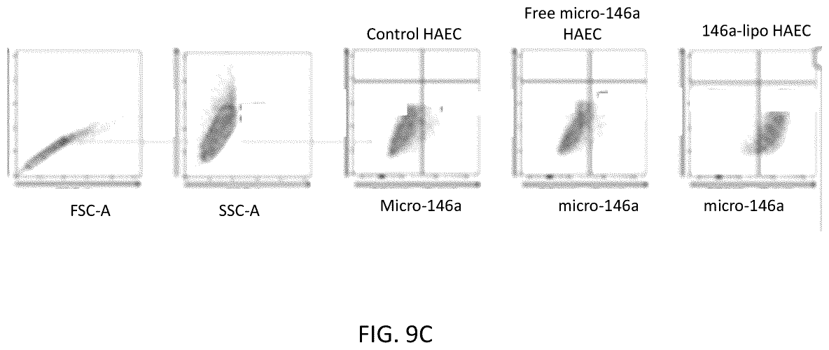

The transfection of microRNA146a-releasing liposomes were studied. As shown in FIGS. 8 and 9, successful transfection of the liposomes was observed using fluorescent microscopy as well as flow cytometry for all three cell types indicating that the liposomes could effectively deliver the miR-146a to the cells (Table 4, FIG. 8). Table 4 shows the Transfection efficiency of free miR-146a+and microRNA146a-releasing liposome.

TABLE 4
Transfection Efficiency of Free
miR-146a and miR-146a Liposomes

| Transfection | Cell Type | | |
|---|---|---|---|
| Efficiency | U937 | | |
| (mean % ± SD %) | Macrophages | HAECs | SMCs |
| Negative Control | 0.03 ± 0.02% | 6.29 ± 0.50% | 0.73 ± 0.41% |
| Free miR-146a | 0.07 ± 0.002% | 5.73 ± 0.41% | 1.36 ± 0.08% |
| 146a-lipo | 96.7 ± 0.66% | 97.2 ± 2.98% | 97.9 ± 0.55% |

The transfection efficiency of the miR-146a encapsulated liposomes for U937 macrophages, HAECs, and SMCs treated with 146a-lipo as determined by flow cytometry were 96.7%, 97.2%, and 97.9% transfected with fluorescent tagged miR-146a, respectively (FIG. 9).

iv. microRNA146a-Releasing Liposome Effect on Foam Cells.

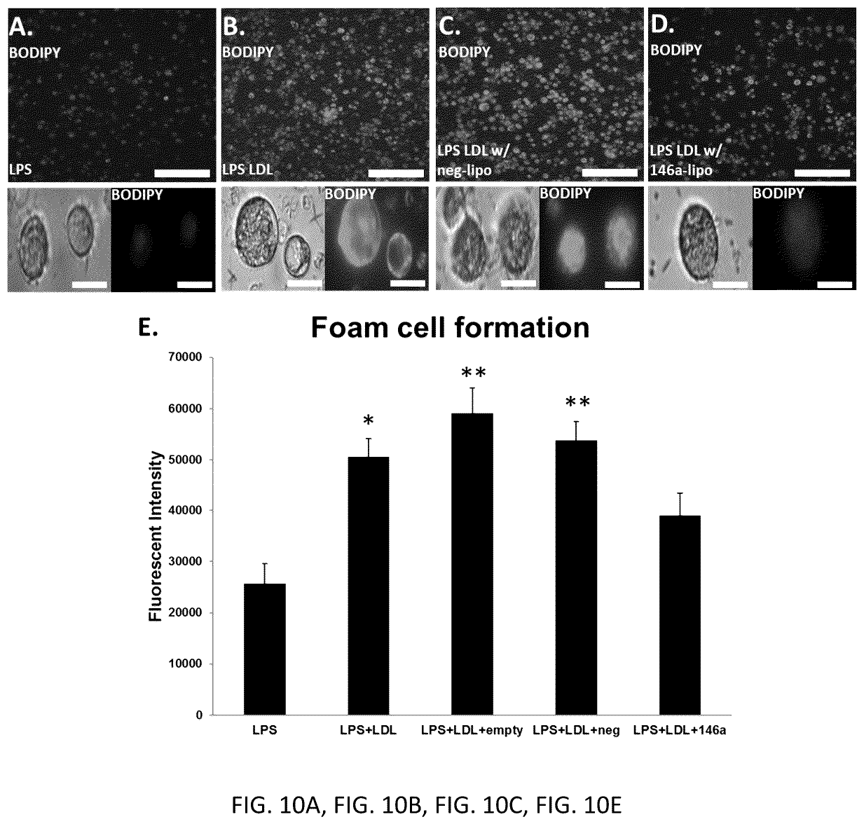

To determine the effect of microRNA146a-releasing liposome on foam cell formation, LPS induced differentiated U937 cells in the presence of ox-LDL were evaluated using BODIPY staining after treatment with liposomes. The microRNA146a-releasing liposome treated differentiated U937 cells showed about 23% less BODIPY staining compared to only LPS-LDL treated differentiated U937 cells. MicroRNA146a-releasing liposome differentiated U937 cells also demonstrated approximately 28% and 24% reduction compared to emp-lipo and neg-lipo differentiated U937 cells respectively (FIG. 10). By effectively reducing foam cell formation, miR-146a loaded liposomes further demonstrate their potential in combatting vascular diseases, particularly atherosclerosis, in which prolonged foam cell formation and lesion formation pose significant health risks.

### 3. Sirolimus-Releasing Liposomes

i. Synthesis of Sirolimus-Releasing Liposomes

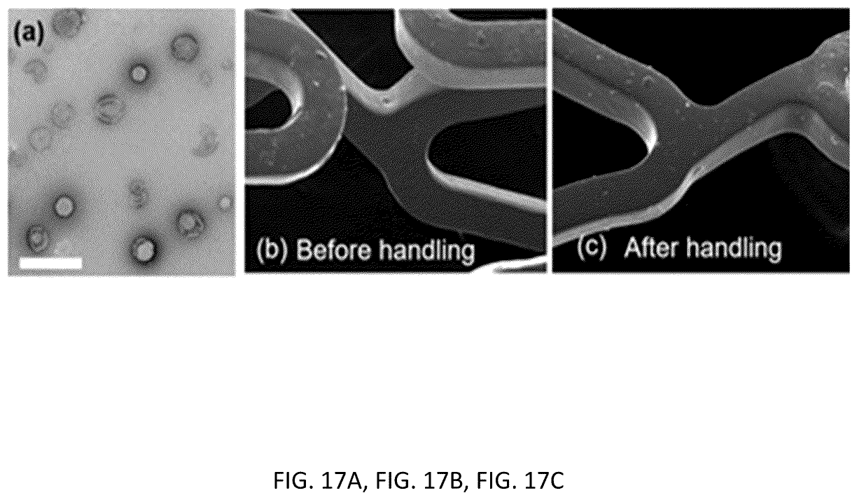

Briefly, DPPC (55%), DSPC-PEG (3%), DOTAP (15%), and CHOL (27%) were dispersed in chloroform and the chloroform was evaporated overnight to create a thin lipid film using a vacuum. The lipid thin film was rehydrated with 0.025M buffer solution for 3 hours to form liposomes. To control size homogeneity, liposomes were then extruded using 800 nm, 400 nm, and 200 nm polycarbonate membrane filters. Then, the liposomes were mixed with sirolimus (1 mg/mL) and sonicated using a water bath for 10 minutes at 55° C. The liposomes were separated from the free, unencapsulated sirolimus using centrifugation (18000 rpm, 20 min). As previously demonstrated, uniform stent coating will be achieved by a rotating ultrasonic spray coating method. Sirolimus encapsulated liposomes will be mixed with PA-YKNO nanomatrix and coated together. The Pt-Cr stent will be coated by the combined prohealing nanomatrix (Sir/NO) using an ultrasonic machine (FIGS. 17B and C).

### 4. Colchicine-Releasing Liposomes

i. Synthesis of Colchicine-Releasing Liposomes

DPPC, DOTAP, and CHOL were dispersed in chloroform in a molar ratio of 3:1:1 in a 3 ml vial. The mixture was then left to dry overnight to create a thin lipid film using a vacuum. The lipid thin film was hydrated with 0.025M HEPES solution for 3 hours to form liposomes. Liposomes were then extruded using 800 nm, 400 nm, and 200 nm polycarbonate membrane filters, respectively, to control size homogeneity. Then, the liposomes were incubated with colchicine (2 mg/mL) and sonicated using a water bath for 10 minutes at 55° C. After that, the solution containing liposomes and sirolimus mixture was incubated at 65° C. overnight.

### 5. Everlolimus-Releasing Liposomes

i. Synthesis of Everolimus-Releasing Liposomes

DPPC, DOTAP, DSPE-PEG, and CHOL were dispersed in chloroform in a molar ratio of 55:25:3:27 in a 3 ml vial. The mixture was then left to dry overnight to create a thin lipid film using a vacuum. Next, the lipid thin film was hydrated with MilliQ water. Liposomes were then extruded using 400 nm and 200 nm polycarbonate membrane filters, respectively, to control size homogeneity. Then, the liposomes were incubated with Everolimus and sonicated using a water bath for 10 minutes at 45° C. The liposomes were allowed to rest for 1 hour before purification. Unencapsulated Everolimus was removed by centrifuging liposomes at 63,000 rpm for 30 minutes.

ii. Release Kinetics of Everolimus-Releasing Liposomes

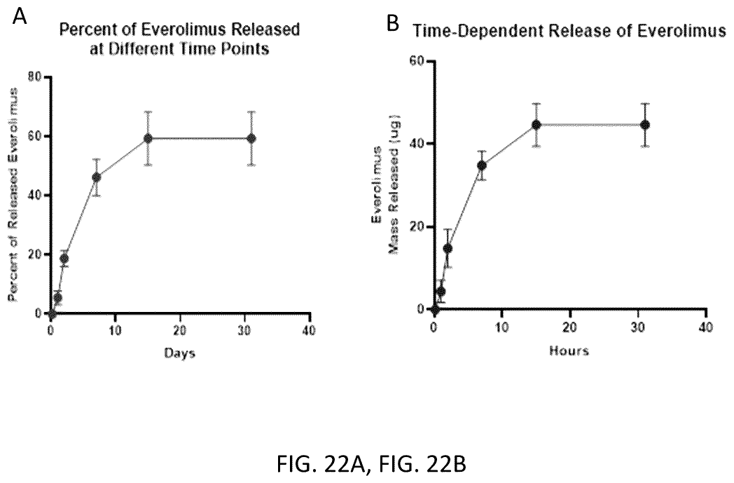

The results show the sustained release profile of Everolimus-releasing liposomes. FIG. 22A demonstrates the percentage of total Everolimus released from the liposomes while FIG. 22B represents the mass of released Everolimus. FIGS. 22A and 22B both demonstrate sustained release as the Everolimus is released rapidly from 0-10 days, gradually from 10-15 days, and stops releasing from 15-30 days. About 60% encapsulated Everolimus (45 μg) was released over 30 days.

iii. Size Analysis of Everlimus-Releasing Liposomes Using DLS

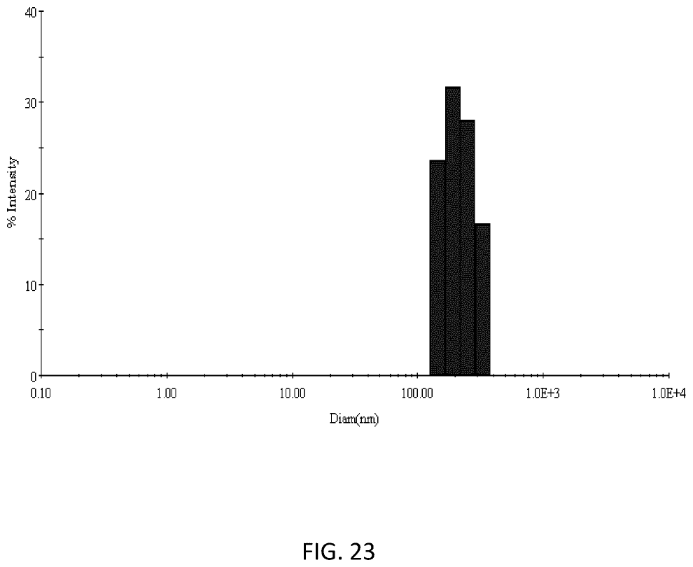

Size and size uniformity of Everlimus-releasing liposomes was assessed using DLS. Everlimus-releasing liposomes were diluted in DI water, and lmL was added to the cuvette (Eppendorf, Hamburg, Germany). The cuvette was loaded into the DLS (DynaPro), and the sample was read at 10% laser power using the Dynamics V6.3.40 software. The radius and polydispersity index (PDI) were recorded to evaluate size and distribution. FIG. 23 demonstrates the distribution of sizes via a logarithmic scale in the Everolimus-releasing liposomes. FIG. 23 shows the Everolimus-releasing liposomes with size of 101 nm±4.4 nm.

### C. Example 3 In Vitro Study and In Vivo Study of PA-C16-YK5-Coated Stent

### i.In Vitro Study of PA-C16-YK5-Coated Stent

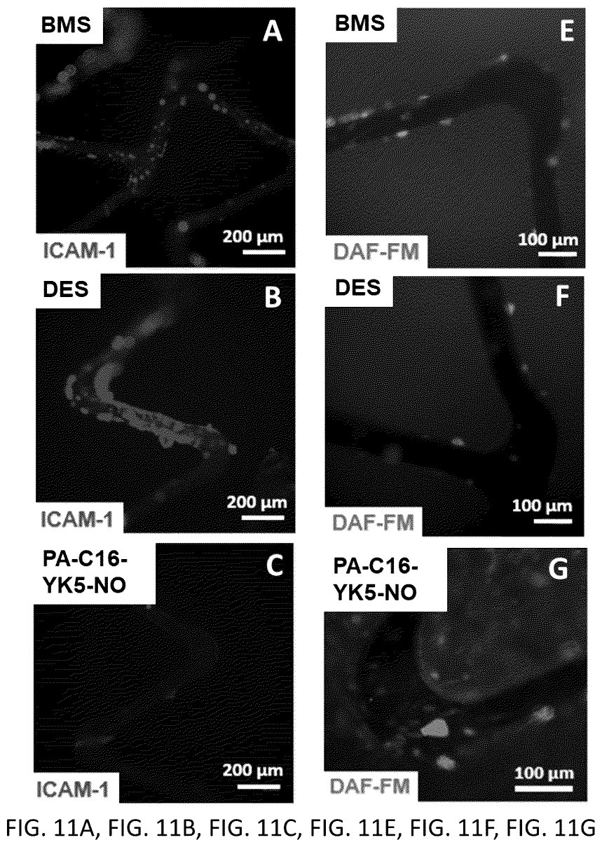

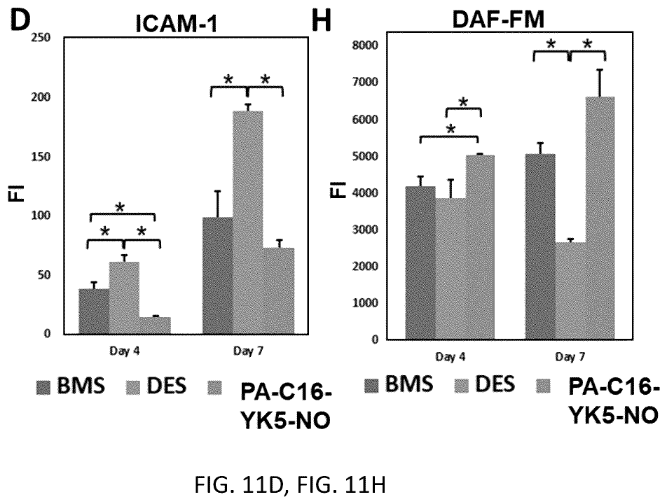

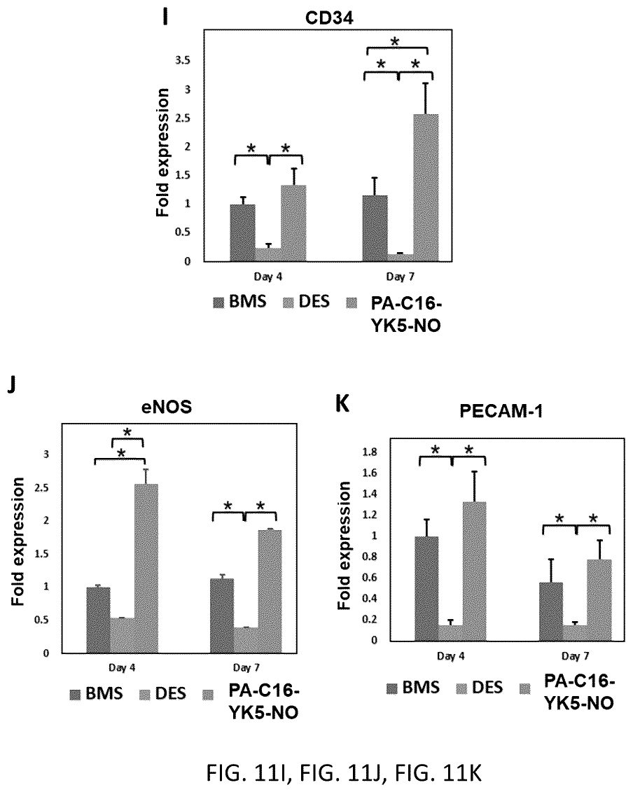

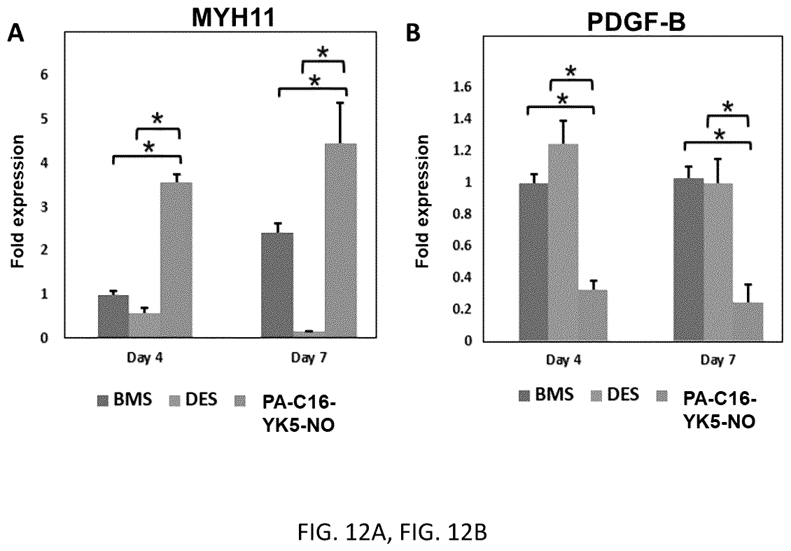

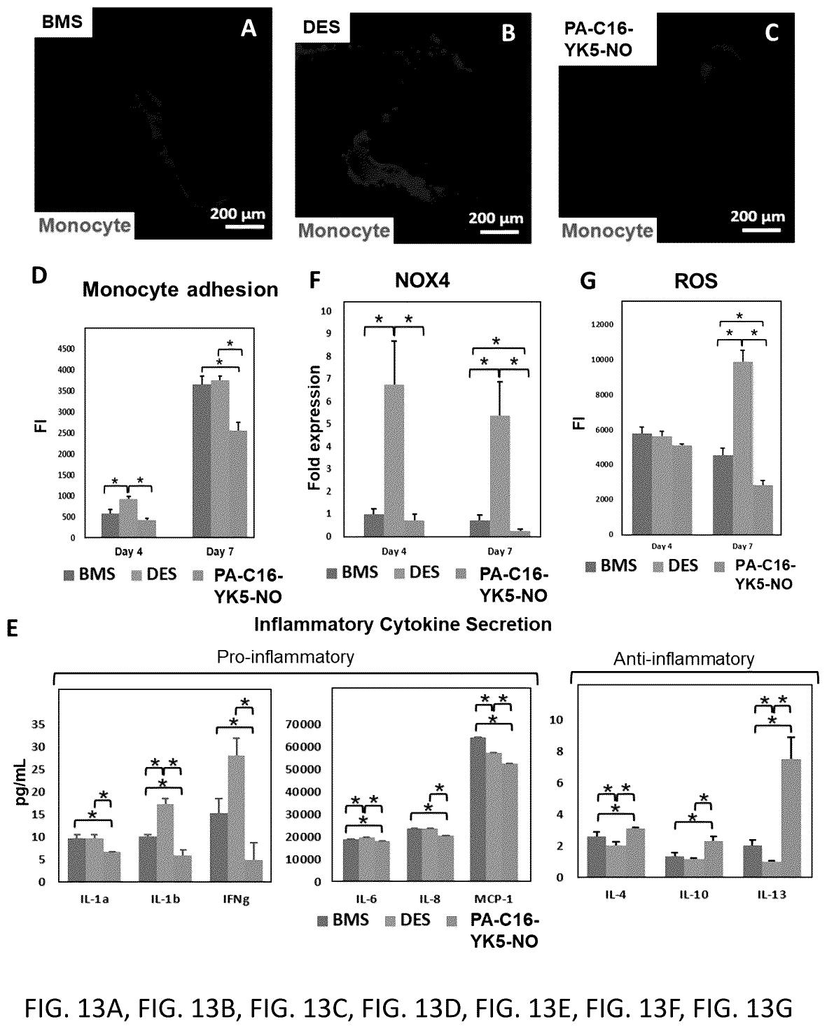

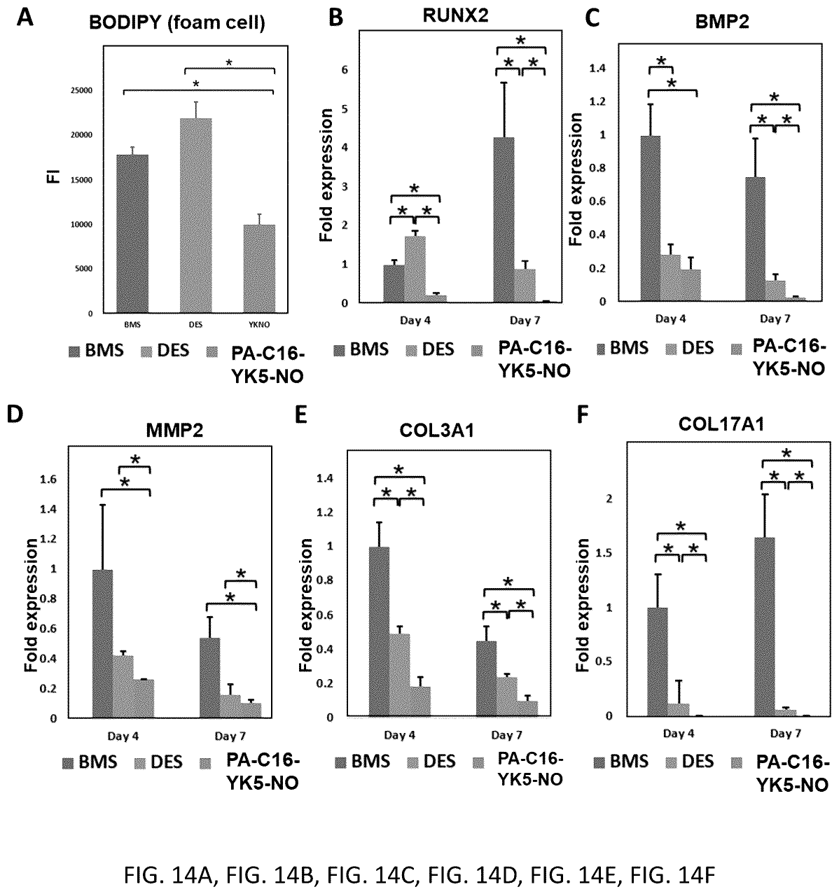

The effect of PA-C16-YK5-coated stent were evaluated on different types of vascular cells associated with atherosclerosis and stenosis using immunostaining, PCR, ELISA, BODIPY staining and DAF staining. The in vitro results using human aortic artery cells showed that compared with commercial-available BMS and DES, the PA-C16-YK5-NO nanomatrix stent coating embrace better therapeutic efficiency. FIG. 11 showed that PA-C16-YK5-NO nanomatrix coated stent improves endothelial functions via increasing function marker expression PECAM-1 and eNOS as well as NO secretion, and decreasing dysfunctional marker ICAM-1 expression. FIG. 12 shows that PA-C16-YK5-NO nanomatrix coated stent promotes the transition of SMC to contractile phenotype by increasing contractile phenotype marker MYH11 expression and decreasing dedifferentiation marker PDGF-B expression. FIG. 13 demonstrated that PA-C16-YK5-NO nanomatrix coated stent reduces inflammation through suppression monocyte adhesion and inflammatory gene expression, while regulating inflammatory cytokine secretion. FIG. 14 showed PA-C16-YK5-NO nanomatrix coated stent administers foam cell formation, ECM remodeling and calcification by regulating related gene expression.

ii. In Vivo Study of PA-C16-YK5-Coated Stent in a Balloon Injury Rabbit Iliac Artery Model:

### 1. Study Design

Three stent cohorts were tested: BMS (Rebel, Boston Scientific), DES (Promus Premier, Boston Scientific), and PA-C16-YK5-NO nanomatrix coated stents (coated Rebel stents, Boston Scientific). A total of seven rabbits (New Zealand white, male and female) were used in this study. After pretreatment with aspirin, each rabbit underwent stent implantation and received two stents, one in each iliac artery, via carotid access using standard catheterization techniques. At termination, stented vessel segments were fixed, extracted, and processed for post-mortem SEM and histological analysis.

i. Rotational Coating Method for Stents

Stents were uniformly coated with PA-C16-YK5-NO using a rotational coating technique through water evaporation-based self-assembly. Bare metal stents (Rebel, Boston Scientific) were mounted on a rotating mandrel attached to a motor and immersed within a PA-C16-YK5-NO solution contained in an open-top reservoir for 12 hours. Critically, the rotation of the stents ensured uniform PA-C16-YK5-NO nanomatrix coating on all stent strut faces, while the open top of the reservoir facilitated solvent evaporation. After rotating in the PA-C16-YK5-NO solution for 12 hours, the stents were allowed to continue rotating out of the solution to dry for 24 hours. Stents were then washed twice with sterile DI water, crimped on the delivery balloon catheter, and ethylene oxide (EtO) sterilized prior to implantation.

ii. Animals

This study was performed at Translation Testing and Training Laboratories (T3 Labs) in accordance with the requirements of the Animal Welfare Act and amendments and in conformance to the standards in the Guide for the Care and the Use of Laboratory Animals. T3 Labs is accredited by the Association for the Assessment and Accreditation of Laboratory Animal Care, International (AAALAC) and registered with the United States Department of Agriculture to conduct research in laboratory animals. All study protocols were reviewed and approved by the Institutional Animal Care and Use Committee (IACUC) prior to study initiation. Male and female New Zealand White rabbits were utilized for this study.

iii. Surgical Procedure

Rabbits were pretreated with aspirin (40 mg/day for three days) and sedated with ketamine (35 mg/kg) and xylazine (5 mg/kg) prior to stent implantation. Anesthesia was maintained with isoflurane (0.5-5%). The carotid access was then surgically prepared, and the animal was heparinized (150 U/kg) to achieve an activated clotting time above 250 seconds. Under fluoroscopic visualization, a guide catheter was placed in the carotid artery and advanced over the aortic arch into the abdominal aorta. A coronary guidewire was then placed in the iliac artery, followed by a PTCA balloon catheter (Emerge, Boston Scientific). The balloon catheter was inflated and deflated three times for 30 seconds with 30-second reflow after each inflation to ensure balloon injury. Following denudation of the target segment, a stent mounted on a delivery balloon was advanced over the guidewire into the iliac artery and deployed over the injured area of the vessel. Stents were deployed as slightly oversized with a stent to artery ratio of 1.1:1. The carotid artery was ligated at the end of the procedure. The contralateral iliac artery underwent stent implantation in a similar manner. Animals received daily anticoagulation treatment with aspirin (40 mg). At termination, animals were euthanized, and iliac arteries were perfusion rinsed with 0.9% saline and then perfusion fixed in situ with 10% neutral buffered formalin. Post perfusion, iliac treatment sites were carefully excised to include unstented areas (˜2-3 cm) proximal and distal to the stented segment. Adjacent muscle and downstream iliac artery segments were also harvested to evaluate for possible systemic inflammation and toxicity. Harvested specimens were immersion fixed in 10% neutral buffered formalin for a minimum of 24 hours prior to off-site shipping for further processing and analysis.

### 2. Result:

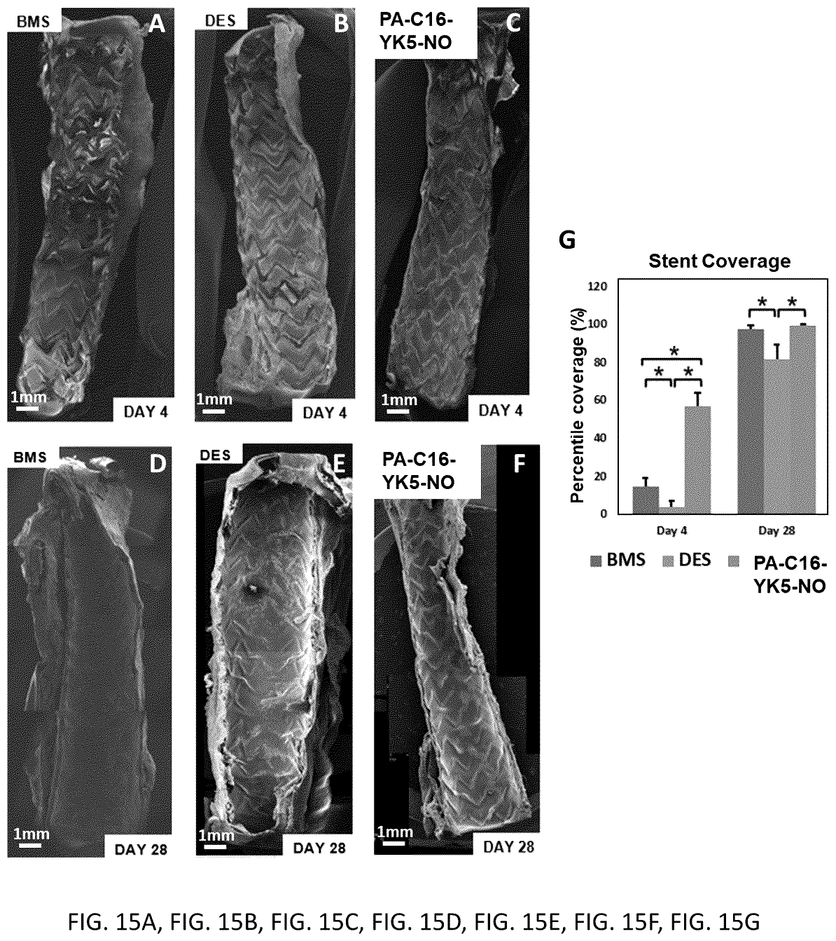

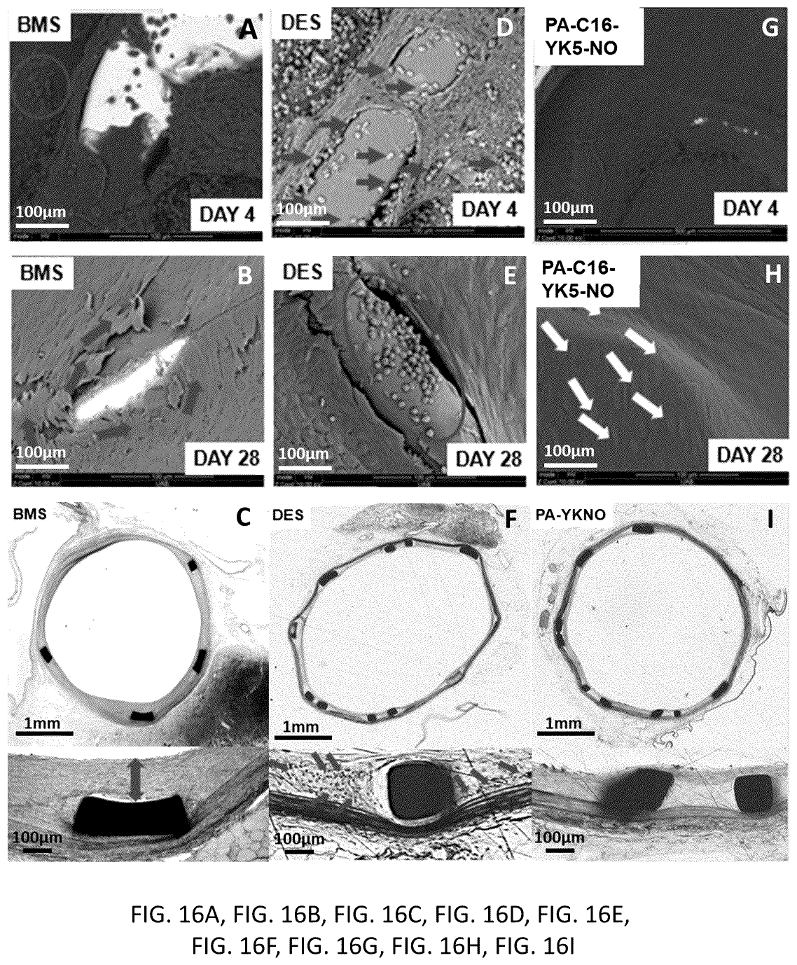

The in vivo results in rabbit iliac artery balloon injury model show that compared with commercial-available BMS and DES, the PA-C16-YK5-NO nanomatrix coated stent have better performance. FIG. 15 shows PA-C16-YK5-NO nanomatrix coated stent could accelerate stent coverage at both day 4 and day 28. FIG. 16 shows PA-C16-YK5-NO nanomatrix coated stent reduces restenosis, suppresses inflammation, and promotes endothelialization.

### D. Example 4 Effects of Colchicine on Cells in Atherosclerosis

### 1. Effects of Colchicine on Foam Cell Formation

Recently, colchicine has been investigated. Foam cells are the hallmark of atherosclerosis. Thus, the effects of colchicine on foam cell formation were investigated.

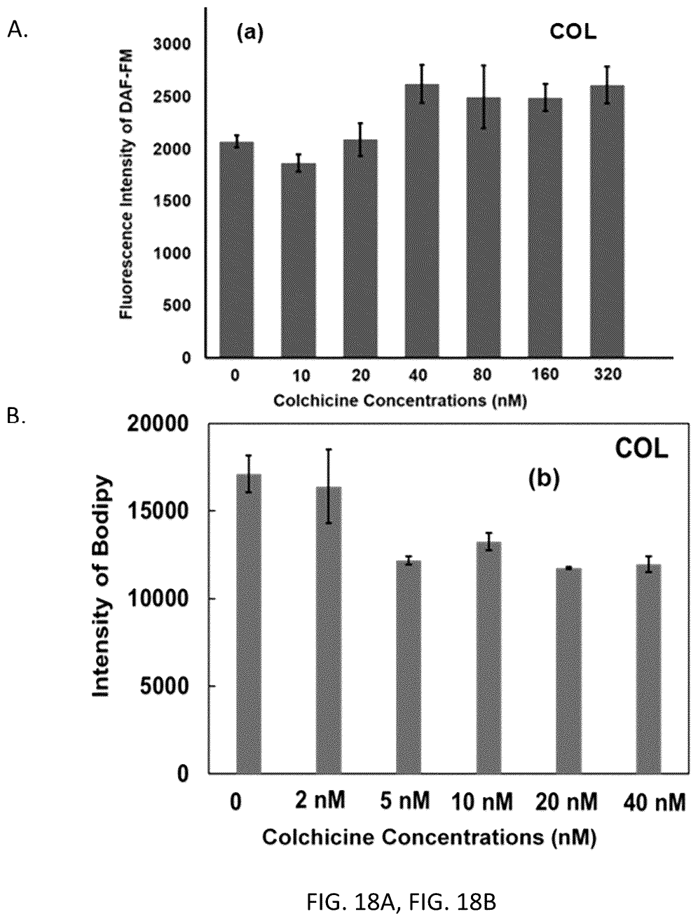

Experimental approach: U937 cells with a cell density of 5000 cell/well were seeded in a 48 well plate, then the colchicine with a selected concentration range from 0 to 40 nM, that is 0, 2 nM, 5 nM, 10 nM, 20 nM, and 40 nM, were added into each well-containing monocytes and RAMPI monocyte culture medium. Then each well were added with M-CSF (100 ng/mL), GM-CSF (25 ng/mL) and INF-γ (100 ng/mL) for macrophage formation. After 7 days of culture, then Ox-LDL (100 ug/mL) was added to each well and cultured for 3 days. After 3 days of culture, the effect of NO on foam cell formation was assessed by using Bodipy. It was shown that the suppression of foam cells was observed when the colchicine concentration is above 2 nM, that is, at the concentrations of 5 nM, 10 nM, 20 nM, and 40 nM (FIG. 18B)

### 2. Effects of Colchicine on Diseased EC Function

Experiment approach: hAECs were seeded in 48-well plate at a cell density of 5000 per well and allowed to attach and grow for 24 h to create a stable confluent monolayer of hAECs (hereafter the “endothelium”). Then, the diseased hAECs (“diseased endothelium”) were obtained by stimulating the hAECs with TNF-α (20 ng/mL) for 18 hours. After that, the colchicine at concentrations of 10 nM, 20 nM, 40 nM, 80 nM, and 160 nM was added to the hAECs after TNF-α stimulation, and then the hAECs were cultured for another three days. Then, the diseased hAEC function was assessed by measuring NO production by hAECs using DAF-FM (4-amino-5-methylamino-2′,7′-difluororescein). DAF-FM is essentially nonfluorescent until they react with NO to form a fluorescent benzotriazole. DAF-FM is cell-permeant and passively diffuses across cellular membranes. Once inside cells, it is de-acetylated by intracellular esterases. The fluorescence quantum yield of DAF-FM is —0.005, but increases about 160-fold, to ˜0.81, after reacting with nitric oxide. With excitation/emission maxima of 495/515 nm, DAF-FM can be detected by any instrument that can detect fluorescein, including fluorescent microplate readers. Particularly, to measure the NO production of hAECs after colchicine treatment, the hAECs were incubated with DAF-FM for 30 minutes at 25° C. After the incubation, the hAECs were watched with buffer and incubated with the buffer for another 15 minutes to allow complete de-esterification of the intracellular diacetates. After the de-esterification, the fluorescence intensity of DAF-FM in hAECs was measured at (excitation/emission) 495/515 nm.

Results: It was shown that when the NO production of the diseased hAECs increased when the colchicine concentration was above 20 nM compared to the diseased hAECs without colchicine treatment, that is, at the concentrations of 40 nM, 80 nM, and 160 nM. The data shown here indicate the potential of colchicine for improving endothelial function (FIG. 18A)

### E. Example 5 The Combining Effect of Sirolimus and NO on ECs

The combining effects of sirolimus and NO on endothelial cell viability using live and dead assay.

### 1. Experiment Approach:

Preparation of NO. The peptide amphiphiles (PA-C16-CA and PA-C16-K5) were synthesized, respectively. These two distinct PAs were mixed in a 9:1 ratio (YIGSR:KKKKK) to form PA-C16-YK5, which was subsequently reacted with NO gas to form PA-YK-NO. The released NO was collected, and NO concentration was measured using Greiss Assay.

Treatment on hAEC. hAECs were seeded in a 48-well plate at a cell density of 2000 per well and allowed to attach for 24 h (hereafter the “endothelium”). For the sirolimus-only group, the sirolimus at concentrations of 0, 0.25, 0.1, 0.5, 1, and 50 nM were added to the hAECs, and then the hAECs were cultured for another three days. Similarly, for the dual-treatment group, sirolimus at similar concentrations was combined with 10 -9 M NO and added to hAEC. Then, the proliferation of hAEC under different conditions was assessed by CyQUANT Cell Proliferation Assay after three days. Briefly, the culture medium in each well was removed, and the cells in the tissue culture plate were frozen overnight at −80° C.; then, the dye in the lysis buffer was added after completely thawing at room temperature. After 30 min incubation, the fluorescent signal was measured using a microplate reader at 485/528 nm.

### 2. Results:

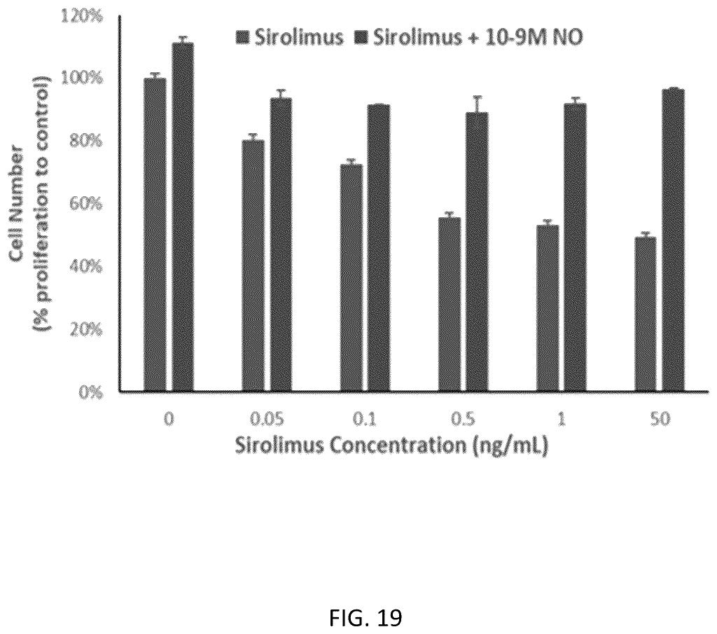

It was shown that hAEC proliferation was decreased with the increasing concentration of sirolimus, while the addition of NO could significantly improve the hAEC proliferation (FIG. 19). Sirolimus reduced the proliferation of hAECs dose-dependently (blue bars). However, dual treatment (red bar, sirolimus, and 10 -9 M NO) CAN significantly improve the proliferation of hAECs.

### F. Example 6 The Combining Effect of Sirolimus and NO on hAoSMCs

The combined effect of sirolimus and NO on SMC proliferation using calcein Am staining and PCNA.

Experiment part: hAoSMCs were seeded in 48 well plate at a cell density of 30,000 cells per cm² and allowed to attach overnight. Then, the SMCs were treated with sirolimus itself (1, 5, 10, and 50 ng/mL) and dual treatment at the same concentrations with nitric oxide (10⁻⁹ M) for 24 hours. SMC proliferation was assessed using a proliferating cell nuclear antigen (PCNA) ELISA kit (abcam). The presence of the PCNA protein was quantitatively assessed using the colorimetric ELISA assay and measured at 450 nm using a standard microplate reader.

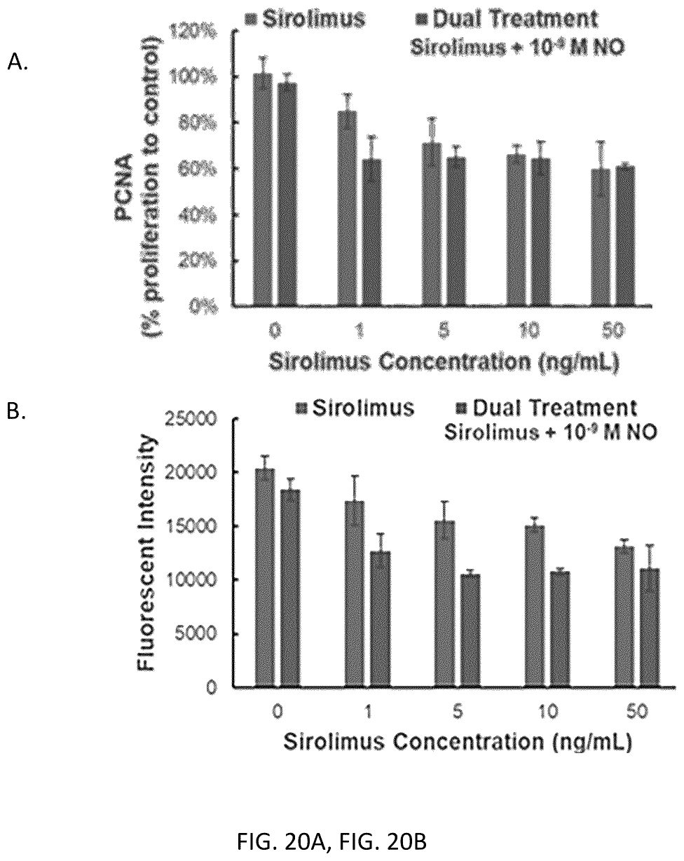

Result: Treatment of 1 ng/mL sirolimus+1 nM NO (FIGS. 20A, 20B, red bar in second column) could obtain the same hAoSMCs suppression as 50 ng/mL sirolimus-only (FIGS. 20A, 20B, blue bar in last column), which greatly decreases the required sirolimus dosage to achieve same hAoSMC suppression.

The combined effect of sirolimus and NO on SMC apoptosis using caspase 3/7.

Experiment part: SMCs were seeded in 48 well plate at a cell density of 30,000 cells per cm2 and allowed to attach overnight. Then, the SMCs were treated with sirolimus itself (1, 5, 10, and 50 ng/mL) and dual treatment at the same concentrations with nitric oxide (10⁻⁹ M) for 24 hours. To verify that sirolimus and nitric oxide reduced cell proliferation and did not affect cell viability, a cell apoptosis assay was utilized under the same conditions. Specifically, caspase 3/7 (CellEvent) was incubated with the live cells for 30 minutes. Capsase-3/7 fluorescent green detection reagent is nonfluorescent due to the presence of DEVD peptide which inhibits the ability of the dye to bind to DNA. However, in apoptotic cells, the DEVD peptide is cleaved, and apoptotic cells will fluoresce. Apoptotic cells were detected and quantified using a plate reader at 530 nm.

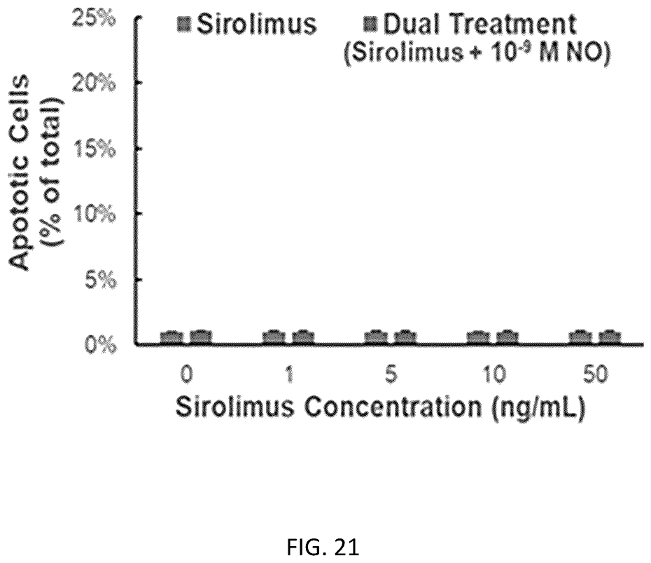

Result: Furthermore, apoptosis was analyzed using Caspase 3,7 (FIG. 21), which showed no apoptosis for any conditions. Thus, dual treatment reduced proliferation but did not affect cell viability at these concentration.

### G. Example 7 Methods for Making Peptide Amphiphile 8 (PA-05-K5-NO)

A method of making peptide amphiphile 8 (PA-05-K5-NO), wherein the step comprises the steps:
a) Synthesis of a hydrophilic peptide comprising a nitric oxide donor sequence. A thirteen-amino acid peptides chain consisting of MMP-2 sensitive sequences (GTAGLIGQ) with NO donating residue KKKKK (K5) were synthesized in solid phase using standard Fmoc chemistry in an Advanced Chemtech Apex 396 peptide synthesizer, as described below. 1) The monomers, which were Fmoc-protected amino acids, were added as triple equivalent solutions in dimethyl formamide to a resin that was linked to the carboxyl terminal amino acid of K (lysine). 2) Them piperidine (three equivalents) was used to deprotect the monomers. 3) Three equivalents of o-benzatriazole-N,N,N′,N′-tetramethyluronium hexafluorophosphate (HBTU) and six equivalents diisopropylethylamine (DIEA) were used to promote the sequential coupling of deprotected amino acids. During the synthesis, dimethyl formamide (DMF) and dichloromethane (DCM) were used as system solvents.
b) Alkylation of the hydrophilic peptide with a hydrophobic moiety. Alkylation was performed by reacting two equivalents of the five-carbon valeric acid with the N-terminal of the peptide in the presence of HBTU and DIEA, thereby creating an amphiphile (PA-05-K5).
c) Cleavage of the PA-05-K5 from resin. The peptide was then cleaved from the resin using 20 mL trifluoroacetic acid (TFA). The acidic solution was dried in a rotary evaporator, and the PA was precipitated in cold ether. The PA precipitates were centrifuged, and the pellet was washed two times with cold ether. The pellet was then dried under vacuum overnight and redissolved in deionized water adjusted to pH 7 by the addition of NaOH.
d) Conjugation of NO to the PA-05-K5. Scrubbed NO gas was reacted with 1 wt % PA-05-K5 solution under argon gas in a 100 mL round bottom flask overnight to obtain the PA-C5-K5-NO.

### H. Embodiments

One embodiment comprises a peptide amphiphile 1 (PA-C16-DS) comprising a hydrophilic peptide sequence and a hydrophobic tail, wherein the hydrophobic tail comprises a moiety having optionally substituted C16 alkyl chain, wherein the hydrophilic peptide sequence comprises a degrading sequence (DS) comprising an amino acid sequence Gly-Thr-Ala-Gly-Leu-ILE-Gly-Gln (GTAGLIGQ; SEQ ID NO:1), wherein the degradation sequence comprises a matrix metalloprotease (MMP) specific cleavage site.

One embodiment comprises a peptide amphiphile 2 (PA-C16-CA) comprises a hydrophilic peptide sequence and a hydrophobic tail, wherein the hydrophobic tail comprises a moiety having optionally substituted C16 alkyl chain, wherein the hydrophilic peptide sequence comprises an endothelial cell-adhesive sequence (CA) comprising an amino acid sequence Tyr-Ile-Gly-Ser-Arg (YIGSR; SEQ ID NO:2) and also comprises a degrading sequence (DS) comprising an amino acid sequence Gly-Thr-Ala-Gly-Leu-ILE-Gly-Gln (GTAGLIGQ; SEQ ID NO:1)

One embodiment comprises a peptide amphiphile 3 (PA-C16-K5) comprises a hydrophilic peptide sequence and a hydrophobic tail, wherein the hydrophobic tail comprises a moiety having optionally substituted C16 alkyl chain, wherein the hydrophilic peptide sequence comprises a nitric oxide producing donor sequence comprising an amino acid sequence Lys-Lys-Lys-Lys-Lys (KKKKK) (K5; SEQ ID NO:3) and also comprises a degrading sequence (DS) comprising an amino acid sequence Gly-Thr-Ala-Gly-Leu-ILE-Gly-Gln (GTAGLIGQ; SEQ ID NO:1), wherein herein one or more of the lysine residues comprise pendant amine groups that react with NO to form a diazeniumdiolate-modified peptide amphiphile, named as peptide amphiphile 4 (PA-C16-K5-NO)

One embodiment comprises a peptide amphiphile 5 (PA-C16-YK) comprises peptide amphiphile 2 (PA-C16-CA) and peptide amphiphile 3 (PA-C16-K5) are at a molar ratio of from about 1:20 to about 20:1, including about 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, or 1:20; wherein herein one or more of the lysine residues comprising pendant amine groups of peptide amphiphile 5 can react with NO to form a diazeniumdiolate-modified peptide amphiphile, named as peptide amphiphile 6 (PA-C16-YK5-NO), also known an endothelium mimicking nanomatrix.

One embodiment comprises a peptide amphiphile 7 (PA-05-K5), comprises a hydrophilic peptide sequence and a hydrophobic tail, wherein the hydrophobic tail comprises a moiety having optionally substituted C5 low alkyl chain; wherein the hydrophilic peptide sequence comprises a nitric oxide producing donor sequence comprising an amino acid sequence Lys-Lys-Lys-Lys-Lys (KKKKK) (K5; SEQ ID NO:3); wherein herein one or more of the lysine residues comprise pendant amine groups that react with NO to form a diazeniumdiolate modified peptide amphiphile, named peptide amphiphile 8 (PA-05-K5-NO).

One embodiment comprises an empty liposome composition comprising cholesterol, and lipid, wherein the lipid is 1,2-distearoyl-snglycero-3-phosphocholine (DSPC), 1,2-dipalmitoyl-snglycero-3-phosphocholine (DPPC), 1, 2-Distearoyl-sn-glycero-3-phosphoethanolamine-poly (ethylene glycol) (DSPE-PEG), or Dioleoyl-3-trimethylammonium propane (DOTAP).

One embodiment is NO-releasing liposomes, wherein the liposomes comprise a diazeniumdiolate modified peptide amphiphile 8 (PA-05-K5-NO), cholesterol, and lipid, wherein the lipid is 1,2-distearoyl-snglycero-3-phosphocholine (DSPC), 1,2-dipalmitoyl-snglycero-3-phosphocholine (DPPC), 1, 2-Distearoyl-sn-glycero-3-phosphoethanolamine-poly (ethylene glycol) (DSPE-PEG), Dioleoyl-3-trimethylammonium propane (DOTAP) dioleoylphosphatidylglycerol (DOPG), dioleoylphosphatidylcholine (DOPC), dimyristoylphosphatidylcholine (DMPC), dioleoylphosphatidylserine (DOPS), palmitoyloleyolphosphatidylglycerol (POPG), dioleoylphosphatidylethanolamine (DOPE), dipalmitoyl phosphatidyl ethanolamine (DPPE), dimyristoylphosphoethanolamine (DMPE), distearoyl-phosphatidy 1-ethanolamine (DSPE), palmitoyloleoyl-phosphatidylethanolamine (POPE), palmitoyloleoylphosphatidylcholine (POPC), egg phosphatidylcholine (EPC), distearoylphosphatidylcholine (DSPC), dipalmitoylphosphatidylcholine (DPPC), dipalmitoylphosphatidylglycerol (DPPG), DSPG, palmitoyloleyolphosphatidylglycerol (POPG), palmitoyloleoyl-phosphatidylethanolamine (POPE), 1-stearoyl-2-oleoylphosphatidyethanolamine (SOPE), DSPE-(polyethylene glycol) PEG, DMPE-PEG, DPPE-PEG or DOPE-PEG; wherein the NO-releasing liposome has a substantially spherical geometry with a diameter between 40 nm to 200 nm.

One embodiment is sirolimus-releasing liposomes, wherein the liposome comprise sirolimus, cholesterol and lipid, wherein the lipid is 1,2-distearoyl-snglycero-3-phosphocholine (DSPC), 1,2-dipalmitoyl-snglycero-3-phosphocholine (DPPC), 1, 2-Distearoyl-sn-glycero-3-phosphoethanolamine-poly (ethylene glycol) (DSPE-PEG), or Dioleoyl-3-trimethylammonium propane (DOTAP); wherein the sirolimus-releasing liposome has a substantially spherical geometry with a diameter of 40 nm to 200 nm.

One embodiment is colchicine-releasing liposomes, wherein the liposome comprise colchicine, cholesterol and lipid, wherein the lipid is 1,2-distearoyl-snglycero-3-phosphocholine (DSPC), 1,2-dipalmitoyl-snglycero-3-phosphocholine (DPPC), 1, 2-Distearoyl-sn-glycero-3-phosphoethanolamine-poly (ethylene glycol) (DSPE-PEG), or Dioleoyl-3-trimethylammonium propane (DOTAP); wherein the colchicine-releasing liposome has a substantially spherical geometry with a diameter between 40 nm to 200 nm.

One embodiment is gentamicin-releasing liposomes, wherein the liposome comprise gentamicin, cholesterol and lipid, wherein the lipid is 1,2-distearoyl-snglycero-3-phosphocholine (DSPC), 1,2-dipalmitoyl-snglycero-3-phosphocholine (DPPC), 1, 2-Distearoyl-sn-glycero-3-phosphoethanolamine-poly (ethylene glycol) (DSPE-PEG), or Dioleoyl-3-trimethylammonium propane (DOTAP); wherein the colchicine-releasing liposome has a substantially spherical geometry with between 40 nm to 200 nm.

One embodiment is vancomycin-releasing liposomes, wherein the liposome comprise vancomycin, cholesterol and lipid, wherein the lipid is 1,2-distearoyl-snglycero-3-phosphocholine (DSPC), 1,2-dipalmitoyl-snglycero-3-phosphocholine (DPPC), 1, 2-Distearoyl-sn-glycero-3-phosphoethanolamine-poly (ethylene glycol) (DSPE-PEG), or Dioleoyl-3-trimethylammonium propane (DOTAP); wherein the colchicine-releasing liposome has a substantially spherical geometry with between 40 nm to 200 nm.

One embodiment is statin-releasing liposome, wherein the liposome comprise colchicine, cholesterol and lipid, wherein the lipid is 1,2-distearoyl-snglycero-3-phosphocholine (DSPC), 1,2-dipalmitoyl-snglycero-3-phosphocholine (DPPC), 1, 2-Distearoyl-sn-glycero-3-phosphoethanolamine-poly (ethylene glycol) (DSPE-PEG), or Dioleoyl-3-trimethylammonium propane (DOTAP); wherein the statin-releasing liposome has a substantially spherical geometry with a diameter between 40 nm to 200 nm.

One embodiment is a composition comprising NO-releasing peptide amphiphile 4 and sirolimus (or liposomal sirolimus).

One embodiment is a composition comprising NO-releasing peptide amphiphile 6 and sirolimus (or liposomal sirolimus).

One embodiment is a composition comprising NO-releasing peptide amphiphile 8 and sirolimus (or liposomal sirolimus).

One embodiment is a composition comprising NO-releasing peptide amphiphile 4 and colchicine (or liposomal colchicine).

One embodiment is a composition comprising NO-releasing peptide amphiphile 6 and colchicine (or liposomal colchicine).

One embodiment is a composition comprising NO-releasing peptide amphiphile 8 and colchicine (or liposomal colchicine).

One embodiment is a composition comprising NO-releasing peptide amphiphile 4 and statin (or liposomal statin).

One embodiment is a composition comprising NO-releasing peptide amphiphile 6 and colchicine (or liposomal statin).

One embodiment is a composition comprising NO-releasing peptide amphiphile 8 and colchicine (or liposomal statin).

One embodiment is a composition comprising NO-releasing peptide amphiphile 4 and gentamicin (or liposomal gentamicin).

One embodiment is a composition comprising NO-releasing peptide amphiphile 6 and gentamicin (or liposomal gentamicin).

One embodiment is a composition comprising NO-releasing peptide amphiphile 8 and gentamicin (or liposomal gentamicin).

One embodiment is a composition comprising NO-releasing peptide amphiphile 4 and vancomycin (or liposomal vancomycin).

One embodiment is a composition comprising NO-releasing peptide amphiphile 6 and vancomycin (or liposomal vancomycin).

One embodiment is a composition comprising NO-releasing peptide amphiphile 8 and vancomycin (or liposomal vancomycin).

One embodiment is a composition comprising NO-releasing peptide amphiphile 4, vancomycin (or liposomal vancomycin) and gentamicin (or liposomal gentamicin).

One embodiment is a composition comprising NO-releasing peptide amphiphile 6, vancomycin (or liposomal vancomycin) and gentamicin (or liposomal gentamicin).

One embodiment is a composition comprising NO-releasing peptide amphiphile 8, vancomycin (or liposomal vancomycin) and gentamicin (or liposomal gentamicin).

One embodiment is a composition comprising a cardiovascular device coated with the composition comprising NO-releasing peptide amphiphile 4 and sirolimus (or liposomal sirolimus), wherein the medical device is a vascular stent, vascular graft, catheter, pacemaker, or heart valve.

One embodiment is a composition comprising a cardiovascular device coated with the composition comprising a NO-releasing peptide amphiphile 6 and sirolimus (or liposomal sirolimus), wherein the medical device is a vascular stent, vascular graft, catheter, pacemaker, or heart valve.

One embodiment is a composition comprising a cardiovascular device coated with the composition comprising NO-releasing peptide amphiphile 8 and sirolimus (or liposomal sirolimus), wherein the medical device is a vascular stent, vascular graft, catheter, pacemaker, or heart valve.

One embodiment is a composition comprising a cardiovascular device coated with the composition comprising a NO-releasing peptide amphiphile 4 and colchicine (or liposomal colchicine) comprising NO-releasing peptide amphiphile 4 and colchicine (or liposomal colchicine), wherein the medical device is a vascular stent, vascular graft, catheter, pacemaker, or heart valve.

One embodiment is a composition comprising a cardiovascular device coated with the composition comprising NO-releasing peptide amphiphile 6 and colchicine (or liposomal colchicine) comprising NO-releasing peptide amphiphile 6 and colchicine (or liposomal colchicine), wherein the medical device is a vascular stent, vascular graft, catheter, pacemaker, or heart valve.

One embodiment is a composition comprising a cardiovascular device coated with the composition comprising NO-releasing peptide amphiphile 8 and colchicine (or liposomal colchicine), wherein the medical device is a vascular stent, vascular graft, catheter, pacemaker, or heart valve.

One embodiment is a composition comprising a cardiovascular device coated with the composition comprising NO-releasing peptide amphiphile 4 and statin (or liposomal statin), wherein the medical device is a vascular stent, vascular graft, catheter, pacemaker, or heart valve.

One embodiment is a composition comprising a cardiovascular device coated with the composition comprising NO-releasing peptide amphiphile 6 and colchicine (or liposomal statin), wherein the medical device is a vascular stent, vascular graft, catheter, pacemaker, or heart valve.

One embodiment is a composition comprising a cardiovascular device coated with the composition comprising NO-releasing peptide amphiphile 8 and colchicine (or liposomal statin), wherein the medical device is a vascular stent, vascular graft, catheter, pacemaker, or heart valve.

One embodiment is a composition comprising a POP coated with the composition comprising NO-releasing peptide amphiphile 4 and gentamicin (or liposomal gentamicin).

One embodiment is a composition comprising a POP coated with the composition comprising NO-releasing peptide amphiphile 6 and gentamicin (or liposomal gentamicin).

One embodiment is a composition comprising a POP coated with the comprising NO-releasing peptide amphiphile 8 and gentamicin (or liposomal gentamicin).

One embodiment is a composition comprising a POP coated with the composition comprising NO-releasing peptide amphiphile 4 and vancomycin (or liposomal vancomycin).

One embodiment is a composition comprising a POP coated with the composition comprising NO-releasing peptide amphiphile 6 and vancomycin (or liposomal vancomycin).

One embodiment is a composition comprising a POP coated with the composition comprising NO-releasing peptide amphiphile 8 and vancomycin (or liposomal vancomycin) comprising NO-releasing peptide amphiphile 8 and vancomycin (or liposomal vancomycin).

One embodiment is a composition comprising a POP coated with the composition comprising NO-releasing peptide amphiphile 4, vancomycin (or liposomal vancomycin) and gentamicin (or liposomal gentamicin).

One embodiment is a composition comprising a POP coated with the composition comprising NO-releasing peptide amphiphile 6, vancomycin (or liposomal vancomycin) and gentamicin (or liposomal gentamicin).

One embodiment is a composition comprising a POP coated with the composition comprising NO-releasing peptide amphiphile 6, vancomycin (or liposomal vancomycin) and gentamicin (or liposomal gentamicin).

One embodiment is a composition comprising peptide amphiphile 6 (PA-C16-YK5-NO), peptide amphiphile 1 (PA-C16-DS), and CaCl2 for preventing or treating arteriovenous fistula failure, wherein the composition takes the form of gel for subject administration.

One embodiment is a composition comprising peptide amphiphile 4 (PA-C16-K5-NO), peptide amphiphile 1 (PA-C16-DS), and CaCl2 for preventing or treating arteriovenous fistula failure, wherein the composition takes the form of gel for subject administration.

One embodiment is a composition comprising peptide amphiphile 8 (PA-C6-K5-NO), peptide amphiphile 1 (PA-C16-DS), and CaCl2 for preventing or treating arteriovenous fistula failure, wherein the composition takes the form of gel for subject administration.

One embodiment is a composition comprising peptide amphiphile 4 (PA-C16-K5-NO), peptide amphiphile 1(PA-C16-DS), CaCl2, and a pharmaceutically active agent for preventing or treating cardiovascular disease, wherein the composition takes the form of gel for administration; wherein the pharmaceutically active agent is sirolimus, everolimus, paclitaxel, colchicine, statin, miRNA or their liposomal drugs including sirolimus, everolimus, paclitaxel, statin, colchicine, or miRNA-releasing liposomes.

One embodiment is a composition comprising peptide amphiphile 6 (PA-C16-YK5-NO), peptide amphiphile 1(PA-C16-DS), CaCl2, and a pharmaceutically active agent for preventing or treating cardiovascular disease, wherein the composition takes the form of gel for administration, wherein the pharmaceutically active agent is sirolimus, everolimus, paclitaxel, colchicine, statins, miRNA, or their liposomal drugs including sirolimus, everolimus, paclitaxel, statin, miRNA, or colchicine-releasing liposomes.

One embodiment is a composition comprising peptide amphiphile 8 (PA-05-K5-NO), peptide amphiphile 1(PA-C16-DS), CaCl2, and a pharmaceutically active agent for preventing or treating cardiovascular disease, wherein the composition takes the form of gel for administration, wherein the pharmaceutically active agent is sirolimus, everolimus, paclitaxel, colchicine, statins, miRNA or their liposomal drugs including sirolimus, everolimus, paclitaxel, statin, miRNA, or colchicine-releasing liposomes.

One embodiment is a method of making peptide amphiphile 8 (PA-05-K5-NO), wherein the step comprises the steps: a) providing a hydrophilic peptide comprising a nitric oxide producing donor sequence, b) alkylating the N-terminus of the hydrophilic peptide with a hydrophobic moiety, wherein the alkylation comprises amination with a valeric acid, c) conjugating NO to the PA-05-K5.

One embodiment is a method comprising making the NO-releasing liposomes, comprising the steps (a) Making lipid thin film comprising cholesterol and lipids, wherein the lipids is chosen from the group consisting of 1,2-distearoyl-snglycero-3-phosphocholine (DSPC), 1,2-dipalmitoyl-snglycero-3-phosphocholine (DPPC), and 1, 2-Distearoyl-sn-glycero-3-phosphoethanolamine-poly (ethylene glycol) (DSPE-PEG), Dioleoyl-3-trimethylammonium propane (DOTAP); (b) Hydrating the lipid thin film with a buffer solution comprising peptide amphiphile 8 (PA-05-K5-NO); (c) Extruding the buffer solution using 800 nm, 400 nm, and 200 nm polycarbonate membrane filters.

1. A peptide amphiphile comprising a hydrophilic peptide sequence and a hydrophobic tail, wherein the hydrophobic tail comprises a moiety having a substituted C5 alkyl chain or a substituted C16 alkyl chain, wherein the hydrophilic peptide sequence comprises a nitric oxide producing donor sequence.

2. The peptide amphiphile of claim 1, wherein the nitric oxide producing donor sequence comprises the amino acid sequence Lys-Lys-Lys-Lys-Lys (KKKKK) (K5; SEQ ID NO:3).

3. The peptide amphiphile of claim 2, wherein the nitric oxide producing donor sequence comprises the amino acid sequence Lys-Lys-Lys-Lys-Lys (KKKKK) (K5; SEQ ID NO:3) and wherein one or more of the Lys residues comprise a pendant amine group.

4. (canceled)

5. (canceled)

6. A composition comprising the peptide amphiphile of claim 1.

7.-11. (canceled)

12. The composition of claim 6, wherein the composition further comprises a pharmaceutically active agent, wherein the pharmaceutically active agent is a therapeutic drug for cardiovascular disease, wherein the therapeutic drug for cardiovascular disease is sirolimus or everolimus, and wherein the peptide amphiphile comprises:
a. a hydrophilic peptide sequence and a hydrophobic tail, wherein the hydrophobic tail comprises a moiety having a substituted C16 alkyl chain, wherein the hydrophilic peptide sequence comprises a nitric oxide producing donor sequence comprising an amino acid sequence Lys-Lys-Lys-Lys-Lys (KKKKK; SEQ ID NO:3) and a degrading sequence (DS) comprising an amino acid sequence Gly-Thr-Ala-Gly-Leu-ILE-Gly-Gln (GTAGLIGQ; SEQ ID NO:1), wherein one or more of the lysine residues comprise a pendant amine group, wherein one or more molecules of nitric oxide is bound to at least one pendant amine group;
b. a hydrophilic peptide sequence and a hydrophobic tail, wherein the hydrophobic tail comprises a moiety having optionally substituted C16 alkyl chain, wherein the hydrophilic peptide sequence comprises a degrading sequence (DS) comprising an amino acid sequence Gly-Thr-Ala-Gly-Leu-ILE-Gly-Gln (GTAGLIGQ; SEQ ID NO:1), and comprises a nitric oxide producing donor sequence comprising an amino acid sequence Lys-Lys-Lys-Lys-Lys (KKKKK) (1(.5; SEQ ID NO:3) and one cell adhesive sequence comprising the amino acid sequence Tyr-Ile-Gly-Ser-Arg (YIGSR; SEQ ID NO:2), wherein one or more of the lysine residues comprise a pendant amine group, wherein one or more molecules of nitric oxide is bound to at least one pendant amine group;
c. a mixture of two peptide amphiphiles,
wherein the first peptide amphiphile comprises a hydrophilic peptide sequence and a hydrophobic tail, wherein the hydrophobic tail comprises a moiety having a substituted C16 alkyl chain, wherein the hydrophilic peptide sequence comprises an endothelial cell-adhesive sequence (CA) comprising an amino acid sequence Tyr-Ile-Gly-Ser-Arg (YIGSR; SEQ ID NO:2) and a degrading sequence (DS) comprising an amino acid sequence Gly-Thr-Ala-Gly-Leu-ILE-Gly-Gln (GTAGLIGQ; SEQ ID NO:1),
wherein the second peptide amphiphile comprises comprises a hydrophilic peptide sequence and a hydrophobic tail, wherein the hydrophobic tail comprises a moiety having optionally substituted C16 alkyl chain, wherein the hydrophilic peptide sequence comprises a nitric oxide producing donor sequence comprising an amino acid sequence Lys-Lys-Lys-Lys-Lys (KKKKK) (K5; SEQ ID NO:3) and also comprises a degrading sequence (DS) comprising an amino acid sequence Gly-Thr-Ala-Gly-Leu-ILE-Gly-Gln (GTAGLIGQ; SEQ ID NO:1),
wherein one or more of the Lys residues of the second peptide amphiphile comprises a pendant amine group, wherein one or more molecules of nitric oxide is bound to at least one pendant amine group; or
d. a hydrophilic peptide sequence and a hydrophobic tail, wherein the hydrophobic tail comprises a moiety having a substituted C5 alkyl chain, wherein the hydrophilic peptide sequence comprises a nitric oxide producing donor sequence, comprising the amino acid sequence Lys-Lys-Lys-Lys-Lys (KKKKK; SEQ ID NO:3) and wherein one or more of the Lys residues comprise a pendant amine group, wherein one or more molecules of nitric oxide is bound to at least one of the pendant amine groups.

13. The composition of claim 6, wherein the composition further comprises a pharmaceutically active agent, wherein the pharmaceutically active agent is a therapeutic drug for cardiovascular disease, wherein the therapeutic drug for cardiovascular disease is colchicine, and wherein the peptide amphiphile comprises:
a. a hydrophilic peptide sequence and a hydrophobic tail, wherein the hydrophobic tail comprises a moiety having a substituted C16 alkyl chain, wherein the hydrophilic peptide sequence comprises a nitric oxide producing donor sequence comprising an amino acid sequence Lys-Lys-Lys-Lys-Lys (KKKKK; SEQ ID NO:3) and a degrading sequence (DS) comprising an amino acid sequence Gly-Thr-Ala-Gly-Leu-ILE-Gly-Gln (GTAGLIGQ; SEQ ID NO:1), wherein one or more of the lysine residues comprise a pendant amine group, wherein one or more molecules of nitric oxide is bound to at least one pendant amine group;
b. a hydrophilic peptide sequence and a hydrophobic tail, wherein the hydrophobic tail comprises a moiety having optionally substituted C16 alkyl chain, wherein the hydrophilic peptide sequence comprises a degrading sequence (DS) comprising an amino acid sequence Gly-Thr-Ala-Gly-Leu-ILE-Gly-Gln (GTAGLIGQ; SEQ ID NO:1), and comprises a nitric oxide producing donor sequence comprising an amino acid sequence Lys-Lys-Lys-Lys-Lys (KKKKK) (1(.5; SEQ ID NO:3) and one cell adhesive sequence comprising the amino acid sequence Tyr-Ile-Gly-Ser-Arg (YIGSR; SEQ ID NO:2), wherein one or more of the lysine residues comprise a pendant amine group, wherein one or more molecules of nitric oxide is bound to at least one pendant amine group;
c. a mixture of two peptide amphiphiles,
wherein the first peptide amphiphile comprises a hydrophilic peptide sequence and a hydrophobic tail, wherein the hydrophobic tail comprises a moiety having a substituted C16 alkyl chain, wherein the hydrophilic peptide sequence comprises an endothelial cell-adhesive sequence (CA) comprising an amino acid sequence Tyr-Ile-Gly-Ser-Arg (YIGSR; SEQ ID NO:2) and a degrading sequence (DS) comprising an amino acid sequence Gly-Thr-Ala-Gly-Leu-ILE-Gly-Gln (GTAGLIGQ; SEQ ID NO:1),
wherein the second peptide amphiphile comprises comprises a hydrophilic peptide sequence and a hydrophobic tail, wherein the hydrophobic tail comprises a moiety having optionally substituted C16 alkyl chain, wherein the hydrophilic peptide sequence comprises a nitric oxide producing donor sequence comprising an amino acid sequence Lys-Lys-Lys-Lys-Lys (KKKKK) (K5; SEQ ID NO:3) and also comprises a degrading sequence (DS) comprising an amino acid sequence Gly-Thr-Ala-Gly-Leu-ILE-Gly-Gln (GTAGLIGQ; SEQ ID NO:1),
wherein one or more of the Lys residues of the second peptide amphiphile comprises a pendant amine group, wherein one or more molecules of nitric oxide is bound to at least one pendant amine group; or
d. a hydrophilic peptide sequence and a hydrophobic tail, wherein the hydrophobic tail comprises a moiety having a substituted C5 alkyl chain, wherein the hydrophilic peptide sequence comprises a nitric oxide producing donor sequence, comprising the amino acid sequence Lys-Lys-Lys-Lys-Lys (KKKKK; SEQ ID NO:3) and wherein one or more of the Lys residues comprise a pendant amine group, wherein one or more molecules of nitric oxide is bound to at least one of the pendant amine groups.

14. The composition of claim 6, wherein the composition further comprises a pharmaceutically active agent, wherein the pharmaceutically active agent is a therapeutic drug for cardiovascular disease, wherein the therapeutic drug for cardiovascular disease is a statin, and wherein the peptide amphiphile comprises:
a. a hydrophilic peptide sequence and a hydrophobic tail, wherein the hydrophobic tail comprises a moiety having a substituted C16 alkyl chain, wherein the hydrophilic peptide sequence comprises a nitric oxide producing donor sequence comprising an amino acid sequence Lys-Lys-Lys-Lys-Lys (KKKKK; SEQ ID NO:3) and a degrading sequence (DS) comprising an amino acid sequence Gly-Thr-Ala-Gly-Leu-ILE-Gly-Gln (GTAGLIGQ; SEQ ID NO:1), wherein one or more of the lysine residues comprise a pendant amine group, wherein one or more molecules of nitric oxide is bound to at least one pendant amine group;
b. a hydrophilic peptide sequence and a hydrophobic tail, wherein the hydrophobic tail comprises a moiety having optionally substituted C16 alkyl chain, wherein the hydrophilic peptide sequence comprises a degrading sequence (DS) comprising an amino acid sequence Gly-Thr-Ala-Gly-Leu-ILE-Gly-Gln (GTAGLIGQ; SEQ ID NO:1), and comprises a nitric oxide producing donor sequence comprising an amino acid sequence Lys-Lys-Lys-Lys-Lys (KKKKK) (1(.5; SEQ ID NO:3) and one cell adhesive sequence comprising the amino acid sequence Tyr-Ile-Gly-Ser-Arg (YIGSR; SEQ ID NO:2), wherein one or more of the lysine residues comprise a pendant amine group, wherein one or more molecules of nitric oxide is bound to at least one pendant amine group;
c. a mixture of two peptide amphiphiles,
wherein the first peptide amphiphile comprises a hydrophilic peptide sequence and a hydrophobic tail, wherein the hydrophobic tail comprises a moiety having a substituted C16 alkyl chain, wherein the hydrophilic peptide sequence comprises an endothelial cell-adhesive sequence (CA) comprising an amino acid sequence Tyr-Ile-Gly-Ser-Arg (YIGSR; SEQ ID NO:2) and a degrading sequence (DS) comprising an amino acid sequence Gly-Thr-Ala-Gly-Leu-ILE-Gly-Gln (GTAGLIGQ; SEQ ID NO:1),
wherein the second peptide amphiphile comprises a hydrophilic peptide sequence and a hydrophobic tail, wherein the hydrophobic tail comprises a moiety having optionally substituted C16 alkyl chain, wherein the hydrophilic peptide sequence comprises a nitric oxide producing donor sequence comprising an amino acid sequence Lys-Lys-Lys-Lys-Lys (KKKKK) (K5;
SEQ ID NO:3) and also comprises a degrading sequence (DS) comprising an amino acid sequence Gly-Thr-Ala-Gly-Leu-ILE-Gly-Gln (GTAGLIGQ; SEQ ID NO:1),
wherein one or more of the Lys residues of the second peptide amphiphile comprises a pendant amine group, wherein one or more molecules of nitric oxide is bound to at least one pendant amine group; or
c. a hydrophilic peptide sequence and a hydrophobic tail, wherein the hydrophobic tail comprises a moiety having a substituted C5 alkyl chain, wherein the hydrophilic peptide sequence comprises a nitric oxide producing donor sequence, comprising the amino acid sequence Lys-Lys-Lys-Lys-Lys (KKKKK; SEQ ID NO:3) and wherein one or more of the Lys residues comprise a pendant amine group, wherein one or more molecules of nitric oxide is bound to at least one of the pendant amine groups.

15. (canceled)

16. (canceled)

17. (canceled)

18. The composition of claim 6, wherein the composition further comprises a pharmaceutically active agent, wherein the pharmaceutically active agent is an antibiotic, wherein the antibiotic is gentamicin and wherein the peptide amphiphile comprises:
a. a hydrophilic peptide sequence and a hydrophobic tail, wherein the hydrophobic tail comprises a moiety having a substituted C16 alkyl chain, wherein the hydrophilic peptide sequence comprises a nitric oxide producing donor sequence comprising an amino acid sequence Lys-Lys-Lys-Lys-Lys (KKKKK; SEQ ID NO:3) and a degrading sequence (DS) comprising an amino acid sequence Gly-Thr-Ala-Gly-Leu-ILE-Gly-Gln (GTAGLIGQ; SEQ ID NO:1), wherein one or more of the lysine residues comprise a pendant amine group, wherein one or more molecules of nitric oxide is bound to at least one pendant amine group;
b. a hydrophilic peptide sequence and a hydrophobic tail, wherein the hydrophobic tail comprises a moiety having optionally substituted C16 alkyl chain, wherein the hydrophilic peptide sequence comprises a degrading sequence (DS) comprising an amino acid sequence Gly-Thr-Ala-Gly-Leu-ILE-Gly-Gln (GTAGLIGQ; SEQ ID NO:1), and comprises a nitric oxide producing donor sequence comprising an amino acid sequence Lys-Lys-Lys-Lys-Lys (KKKKK) (1(5; SEQ ID NO:3) and one cell adhesive sequence comprising the amino acid sequence Arg-Gly-Asp-Ser (RGDS; SEQ ID NO:5), wherein one or more of the Lys residues of the second peptide amphiphile comprises a pendant amine group, wherein one or more molecules of nitric oxide is bound to at least one pendant amine group;
c. PA-C16-K5-YIGSR-NO; a hydrophilic peptide sequence and a hydrophobic tail, wherein the hydrophobic tail comprises a moiety having optionally substituted C16 alkyl chain, wherein the hydrophilic peptide sequence comprises a nitric oxide producing donor sequence comprising an amino acid sequence Lys-Lys-Lys-Lys-Lys (KKKKK) (K5; SEQ ID NO:3) and comprises a degrading sequence (DS) comprising an amino acid sequence Gly-Thr-Ala-Gly-Leu-ILE-Gly-Gln (GTAGLIGQ; SEQ ID NO:1), and further comprising a cell adhesive ligand comprising an amino acid sequence Tyr-Ile-Gly-Ser-Arg (YIGSR; SEQ ID NO:2), wherein herein one or more of the lysine residues comprise pendant amine groups that react with NO to form a diazeniumdiolate-modified peptide amphiphile;
d. a mixture of two peptide amphiphiles,
wherein the first peptide amphiphile comprises a hydrophilic peptide sequence and a hydrophobic tail, wherein the hydrophobic tail comprises a moiety having a substituted C16 alkyl chain, wherein the hydrophilic peptide sequence comprises an endothelial cell-adhesive sequence (CA) comprising an amino acid sequence Tyr-Ile-Gly-Ser-Arg (YIGSR; SEQ ID NO:2) and a degrading sequence (DS) comprising an amino acid sequence Gly-Thr-Ala-Gly-Leu-ILE-Gly-Gln (GTAGLIGQ; SEQ ID NO:1),
wherein the second peptide amphiphile comprises a hydrophilic peptide sequence and a hydrophobic tail, wherein the hydrophobic tail comprises a moiety having optionally substituted C16 alkyl chain, wherein the hydrophilic peptide sequence comprises a nitric oxide producing donor sequence comprising an amino acid sequence Lys-Lys-Lys-Lys-Lys (KKKKK) (K5;
SEQ ID NO:3) and also comprises a degrading sequence (DS) comprising an amino acid sequence Gly-Thr-Ala-Gly-Leu-ILE-Gly-Gln (GTAGLIGQ; SEQ ID NO:1),
wherein one or more of the Lys residues of the second peptide amphiphile comprises a pendant amine group, wherein one or more molecules of nitric oxide is bound to at least one pendant amine group; or
e. a hydrophilic peptide sequence and a hydrophobic tail, wherein the hydrophobic tail comprises a moiety having a substituted C5 alkyl chain, wherein the hydrophilic peptide sequence comprises a nitric oxide producing donor sequence, comprising the amino acid sequence Lys-Lys-Lys-Lys-Lys (KKKKK; SEQ ID NO:3) and wherein one or more of the Lys residues comprise a pendant amine group, wherein one or more molecules of nitric oxide is bound to at least one of the pendant amine groups.

19. The composition of claim 6, wherein the composition further comprises a pharmaceutically active agent, wherein the pharmaceutically active agent is an antibiotic, wherein the antibiotic is vancomycin and wherein the peptide amphiphile comprises:
a. a hydrophilic peptide sequence and a hydrophobic tail, wherein the hydrophobic tail comprises a moiety having a substituted C16 alkyl chain, wherein the hydrophilic peptide sequence comprises a nitric oxide producing donor sequence comprising an amino acid sequence Lys-Lys-Lys-Lys-Lys (KKKKK; SEQ ID NO:3) and a degrading sequence (DS) comprising an amino acid sequence Gly-Thr-Ala-Gly-Leu-ILE-Gly-Gln (GTAGLIGQ; SEQ ID NO:1), wherein one or more of the lysine residues comprise a pendant amine group, wherein one or more molecules of nitric oxide is bound to at least one pendant amine group;
b. a hydrophilic peptide sequence and a hydrophobic tail, wherein the hydrophobic tail comprises a moiety having optionally substituted C16 alkyl chain, wherein the hydrophilic peptide sequence comprises a degrading sequence (DS) comprising an amino acid sequence Gly-Thr-Ala-Gly-Leu-ILE-Gly-Gln (GTAGLIGQ; SEQ ID NO:1), and comprises a nitric oxide producing donor sequence comprising an amino acid sequence Lys-Lys-Lys-Lys-Lys (KKKKK) (1(.5; SEQ ID NO:3) and one cell adhesive sequence comprising the amino acid sequence Arg-Gly-Asp-Ser (RGDS; SEQ ID NO:5), wherein one or more of the Lys residues of the second peptide amphiphile comprises a pendant amine group, wherein one or more molecules of nitric oxide is bound to at least one pendant amine group;
c. a hydrophilic peptide sequence and a hydrophobic tail, wherein the hydrophobic tail comprises a moiety having optionally substituted C16 alkyl chain, wherein the hydrophilic peptide sequence comprises a degrading sequence (DS) comprising an amino acid sequence Gly-Thr-Ala-Gly-Leu-ILE-Gly-Gln (GTAGLIGQ; SEQ ID NO:1), and comprises a nitric oxide producing donor sequence comprising an amino acid sequence Lys-Lys-Lys-Lys-Lys (KKKKK) (K5; SEQ ID NO:3) and one cell adhesive sequence comprising the amino acid sequence Tyr-Ile-Gly-Ser-Arg (YIGSR; SEQ ID NO:2), wherein one or more of the Lys residues of the second peptide amphiphile comprises a pendant amine group, wherein one or more molecules of nitric oxide is bound to at least one pendant amine group;
d. a mixture of two peptide amphiphiles,
wherein the first peptide amphiphile comprises a hydrophilic peptide sequence and a hydrophobic tail, wherein the hydrophobic tail comprises a moiety having a substituted C16 alkyl chain, wherein the hydrophilic peptide sequence comprises an endothelial cell-adhesive sequence (CA) comprising an amino acid sequence Tyr-Ile-Gly-Ser-Arg (YIGSR; SEQ ID NO:2) and a degrading sequence (DS) comprising an amino acid sequence Gly-Thr-Ala-Gly-Leu-ILE-Gly-Gln (GTAGLIGQ; SEQ ID NO:1),
wherein the second peptide amphiphile comprises comprises a hydrophilic peptide sequence and a hydrophobic tail, wherein the hydrophobic tail comprises a moiety having optionally substituted C16 alkyl chain, wherein the hydrophilic peptide sequence comprises a nitric oxide producing donor sequence comprising an amino acid sequence Lys-Lys-Lys-Lys-Lys (KKKKK) (K5; SEQ ID NO:3) and also comprises a degrading sequence (DS) comprising an amino acid sequence Gly-Thr-Ala-Gly-Leu-ILE-Gly-Gln (GTAGLIGQ; SEQ ID NO:1),
wherein one or more of the Lys residues of the second peptide amphiphile comprises a pendant amine group, wherein one or more molecules of nitric oxide is bound to at least one pendant amine group; or
e. a hydrophilic peptide sequence and a hydrophobic tail, wherein the hydrophobic tail comprises a moiety having a substituted C5 alkyl chain, wherein the hydrophilic peptide sequence comprises a nitric oxide producing donor sequence, comprising the amino acid sequence Lys-Lys-Lys-Lys-Lys (KKKKK; SEQ ID NO:3) and wherein one or more of the Lys residues comprise a pendant amine group, wherein one or more molecules of nitric oxide is bound to at least one of the pendant amine groups.

20.-34. (canceled)

35. A gel comprising the peptide amphiphile of claim 1.

36. A medical device coated with a peptide amphiphile of claim 1.

37. — 39. (canceled)

40. A method of preventing infection in a subject comprising administering to the subject a therapeutically effective amount of a composition of claim 6, wherein the composition further comprises a pharmaceutically active agent, wherein the pharmaceutically active agent is an antibiotic to a subject in need thereof.

41. A method of treating a subject in need thereof comprising administering the medical device of claim 36 to a subject during osseointegration.

42. A method of increasing vascularization or treating inflammation in a subject comprising administering the gel of claim 35 to a subject in need thereof.

43. (canceled)

44. A method of making the peptide amphiphile of claim 2, wherein the pendant amine groups can react with nitric oxide to form a diazeniumdiolate-modified peptide amphiphile, wherein the diazeniumdiolate is a NO donor and comprises one or more molecule of nitric oxide comprising:
a. obtaining a peptide amphiphile comprising a nitric oxide (NO) producing donor sequence, wherein the peptide amphiphile comprises an N-terminus,
b. alkylating the N-terminus of the peptide amphiphile with a hydrophobic moiety, wherein the alkylation comprises amination with a valeric acid, and
c. conjugating NO to the peptide amphiphile.

45.-48. (canceled)

49. A method of treating arteriovenous fistula failure or preventing or treating cardiovascular disease in a subject, comprising administering to the subject a therapeutically effective amount of the peptide amphiphile of claim 1 to a subject in need thereof.

50.-53. (canceled)

54. A kit comprising the peptide amphiphile of claim 1.

55. A gel comprising a peptide amphiphile, wherein the peptide amphiphile a hydrophilic peptide sequence and a hydrophobic tail, wherein the hydrophobic tail comprises a moiety having an optionally substituted C16 alkyl chain, wherein the hydrophilic peptide sequence comprises a nitric oxide producing donor sequence comprising an amino acid sequence KKKKK (SEQ ID NO:3) and also comprises a degrading sequence (DS) comprising an amino acid sequence GTAGLIGQ (SEQ ID NO:1), wherein one or more of the Lys residues of the peptide amphiphile comprises a pendant amine group, wherein one or more molecules of nitric oxide is bound to at least one pendant amine group.

56. (canceled)

57. A composition comprising a peptide amphiphile, wherein the peptide amphiphile comprises a hydrophilic peptide sequence and a hydrophobic tail, wherein the hydrophobic tail comprises a moiety having a substituted a substituted C16 alkyl chain, wherein the hydrophilic peptide sequence comprises a nitric oxide producing donor sequence wherein the nitric oxide producing donor sequence comprises the amino acid sequence Lys-Lys-Lys-Lys-Lys (KKKKK) (K5; SEQ ID NO:3) and a degrading sequence (DS) comprising an amino acid sequence Gly-Thr-Ala-Gly-Leu-ILE-Gly-Gln (GTAGLIGQ; SEQ ID NO:1), wherein one or more of the lysine residues comprise a pendant amine group, wherein one or more molecules of nitric oxide is bound to at least one pendant amine group.

58. The peptide Amphiphile of claim 1, further comprising a degrading sequence (DS) comprising an amino acid sequence Gly-Thr-Ala-Gly-Leu-ILE-Gly-Gln (GTAGLIGQ; SEQ ID NO:1).
